(19) United States
(12) Patent Application Publication
(10) Pub. No.: US 2024/0026471 A1
(43) Pub. Date: Jan. 25, 2024

(54) MOLECULAR ELECTRONIC SENSORS FOR MULTIPLEX GENETIC ANALYSIS USING DNA REPORTER TAGS

(71) Applicant: Roswell Biotechnologies, Inc., San Diego, CA (US)

(72) Inventors: Barry Merriman, San Diego, CA (US); Carl Fuller, Berkeley Heights, NJ (US); Paul Mola, San Diego, CA (US); Hadi Abderrahim, San Diego, CA (US); Pius S. Padayatti, San Diego, CA (US)

(73) Assignee: Roswell Biotechnologies, Inc., San Diego, CA (US)

(21) Appl. No.: 18/010,451

(22) PCT Filed: Jun. 15, 2021
(86) PCT No.: PCT/US2021/037469

§ 371 Date: Jun. 19, 2023

Related U.S. Application Data
(60) Provisional application No. 63/039,337, filed on Jun. 15, 2020.

(51) Int. Cl. C12Q 1/70; C12Q 1/6825

(52) U.S. Cl. CPC C12Q 1/701; C12Q 1/6825

(57) ABSTRACT

Biomolecular sensors and methods of manufacturing these sensors are provided. An exemplary sensor has a source electrode, a drain electrode spaced apart from the source electrode by a sensor gap, a gate electrode, wherein the source, drain and gate electrodes cooperate to form an electrode circuit, a bridge bridging across said sensor gap coupling the source and drain electrodes, and a probe coupled to the bridge, wherein the probe comprises nucleic acid and interaction of the nucleic acid probe with a target nucleic acid is detectable by monitoring at least one parameter of the electrode circuit. Methods of detecting a target nucleic acid are also provided. Preferred target nucleic acids that are detected are from pathogens, such as coronavirus.

### CROSS REFERENCE TO RELATED APPLICATIONS

This application is a National Phase of PCT/US21/37469 filed on Jun. 15, 2021, which claims priority to U.S. provisional application having Ser. No. 63/039,337 by Barry Merriman et al., filed on Jun. 15, 2020, and entitled ‘Molecular Electronic Sensors for Multiplex Genetic Analysis using DNA Reporter Tags,’ both of which are incorporated by reference herein.

### SEQUENCE LISTINGS

Filed herewith and expressly incorporated herein by reference in its entirety is a Sequence Listing submitted electronically as an ASCII text file via EFS-WEB. The ASCII copy, created on ______, is named______ and is ______ bytes in size.

### FIELD

This disclosure is in the field of sensors, and also in the field of nano-electronics. More specifically, it is in the field of molecular electronic sensors. It is also in the field of genetic analysis, and more specifically, in the field of measuring samples for their content of specific DNA molecules of interest. It is also in the field of infectious disease monitoring, and in particular the detection, monitoring or diagnosis of viral infectious diseases. In particular, this includes monitoring of viral disease such as COVID-19.

### BACKGROUND

In the field of genetic analysis, it is important to be able to determine if a given sample of biological material contains a target DNA or RNA segment of interest. Another import example is species identification, where a sequence characteristic of species, is searched for within the sample. This is, for example, important for the environmental monitoring of, and diagnosis of, infectious disease. For example, a DNA segment that identifies a pathogen, such as a parasite, bacteria or virus, can be looked for within the sample taken from the environment, or from an animal or person that may be infected. This is especially important for the environmental surveillance and epidemiology of viral diseases with the potential for large scale, rapidly progressing infection or pandemics, such as COVID-19. It is also important in genetic analysis to look for known genetic variants that may occur, relative to a give segment of DNA. This type of measurement is known as genotyping in the context of looking for known variants in humans and animals. In the context of pathogens, this often takes the form of identification of strains, which are defined by DNA or RNA variants relative to a reference genome sequence, or by the sequence differences between two genomes.

It is also important to be able to determine the concentration level of a DNA or RNA segment of interest in a sample. One such example is in gene expression analysis, where the activity level or expression level of genes, represented in the form of messenger RNA, can be assessed in a sample. This is important, for example, in studying gene function, or in characterizing the pathology of cancers for research, diagnosis and treatment. Another such example is in Non-Invasive Pregnancy Testing (NIPT), which requires measurement of levels of non-maternal cell-free DNA fragments in blood samples. Another similar example is Liquid Biopsy, for early detection or recurrence monitoring of cancer, which may look to detect of the levels of known mutant sequences in blood samples. Another example is Comparative Genomic Hybridization (CGH), where the relative concentration of segment of genomic DNA in a sample is used to detect genomic duplication or deletion events, both in diagnosing germline disease such as Down Syndrome (Trisomy 21), or in characterizing genomic alterations in cancers as a component of Precision Medicine for Oncology. Another example arises in the field called metagenomics, where the goal is to characterize complex populations of diverse organisms present in an environmental sample, such as a soil or water sample, by extracting and quantifying the abundance of different forms of genomic DNA present in the organisms in the sample. Of particular interest for health and disease is the special case of assessing microbiomes, such as gut microbiome, or oral microbiome, for the populations of bacteria present. For the purpose of quantifying such complex populations, one common approach is to use PCR to target a common “barcode of life” DNA segment that is present in all the organisms of interest, and has enough diversity to distinguish species and strains of interest, an in this approach, the focus becomes identifying and measuring the relative concentrations of these fragments.

These general classes of genetic analysis problems-measuring the presence of or concentration of DNA segments of interest—have been addressed using well known modern molecular biology tools such as PCR, DNA Microarrays, and DNA sequencing. Older techniques predating these, such as Southern Blots (for measuring DNA targets) and Northern Blots (for measuring RNA targets) have also been applied to these problems. All such assays, including Southern and Northern Blots, employ the process of DNA “hybridization” as a fundamental part of the detection scheme.

As shown in FIG. 2A, hybridization is the natural phenomena where, a single strand of DNA will, with high efficiency, bind to its specific reverse complementary strand in a solution. In FIG. 2B, DNA is depicted in cartoon fashion to illustrate the logical structure as a sequence of bases, with the important backbone orientations (3′ and 5′ ends) indicated, and to indicate the logical Watson-Crick Base Pairings (A-T, G-C in DNA, as well as (not shown) U-T in RNA-RNA or RNA-DNA pairings), but the figure does not indicate the double-helix physical structure of the duplex, or the chemical hydrogen bonds involved in base pairing. The reverse process, where a duplex separates into independent single strands, is referred to as dissociation or melting, and it is a strongly temperature dependent process, the transition temperature being known as the melting temperature (Tm, which depends on the solution composition as well, and to a lesser extent on the strand concentration, and which can also depend on other chemical, steric or entropy effects like the proximity of other molecules, or surfaces, or the tethering of DNA to other molecules). For DNA free in solution, substantially below the melting temperature, the duplex form is stable and long lived, and substantially above the melting point, it is unstable and short lived. Within a few degrees C. of the melting point, the on and off reactions are both common. Generally, hybridization is a chemical reaction process obeying Boltzmann distribution kinetics, and as such it is a reversible process, for which the precise definition of melting point is the temperature where strands are equally likely to be bound and unbound for the given solution conditions. In common physiological solution conditions, melting point for duplexes shorter than 10 bases may be below room temperature (25C), while for duplexes 50 bases or longer, or with higher G-C pair content, Tm may approach the boiling point of water (100C). In typical buffer solutions used in molecular biology, the salt levels and divalent cation levels (such as Mg++) have moderate effect on the melting point, and therefore the kinetics of hybridization and dissociation. At low concentration of complementary strands, it may take a long time for duplex formation to occur, as the strands must meet first, primarily by diffusive transport.

Similar to what is shown in FIG. 2A for DNA, any combination of complementary DNA and RNA strands can pair by hybridization in this way, as can strands of DNA or RNA that contain various nucleotide analogs such as LNA or PNA, or various chemically modified nucleotide. Longer segments of DNA that match along a segment will form a duplex pair along that segment, even if the ends do not match, although the free ends may result in a slightly lower melting point. Strands that are not perfectly matched can bind, but the presence of mis-paired bases substantially lowers the melting point, and they may not be stable under many conditions of interest. This relatively lower Tm for mis-paired strands (sometimes called cross-hybridization) can be used advantageously to design detection assays using hybridization, such that an exact match is preserved and produces and enduring detectable signal, while all the great many possible mismatch interactions with off-target fragments can be eliminated from producing signals, or otherwise result in signals that are substantially below some form of detection threshold. The well-known Southern Blot assay relies on DNA-DNA hybridization, and the Northern Blot assay relies on DNA-RNA or RNA-RNA hybridization, for the critical part of the sequence-specific detection process. DNA Microarrays, which were a technological descendent of Southern and Northern Blots, also rely on DNA hybridization as the critical part of their sequence-specific detection process. Indeed, even PCR reactions rely in part on the hybridization process, as the single stranded DNA oligo primers used in PCR and related reactions must stably hybridize to a single stranded template in a sequence-specific manner, free of mismatches, in order for the polymerase to efficiently extend the primer. Similarly, many forms of DNA sequencing processes also utilize some form of priming and polymerase extension as a part of the overall process. In these particular contexts involving priming for polymerases, the hybridization is often referred to as primer binding, or primer-template binding.

Molecular electronics is a general field of technology in which single molecules are placed as a component in an electrical circuit, to perform some useful electrical functions such as transducing some chemical or molecular event into an electrical signal. This general concept is illustrated in FIG. 1, which shows a cartoon depiction of a single molecule that has been bound to two nano-scale electrodes, and where a surrounding circuit is used to apply a voltage, V, across the electrodes, and to measure the current, i, that flows through the electrodes and molecule, over time. When such a molecule interacts with other molecules, these events may be transduced into signals or detectible signatures present in the measured current versus time trace. The resulting system has the potential to be used as a sensor for a great variety of molecular interaction processes. As highlighted in the cartoon, the electrodes could be made of metal or of semiconductor materials, and the molecule may be bound in place to the electrodes using various attached binding or conjugation groups, or by various binding reactions. For biosensor applications, this device typically must be able to operate as a sensor when immersed in a solution phase, particularly an aqueous solution. The scale of the gap between electrodes is set based on the length of the molecules of interest, but is typically on the scale of nanometers (nm), or 10's of nm or at most 100's of nm. As a result, molecular electronic devices readily scale to the smallest possible dimensions for electrical circuits—the scale of molecules. Molecular electronics therefore offers the potential to make semiconductor chip-based, all-electronic sensor devices that are both maximally scalable and maximally sensitive, with single molecule detection capabilities. This is an emerging field of technology, with much promise, but it remains extremely challenging both to devise and to fabricate such systems that actually perform applications of interest.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

It is the object of this invention to disclose and provide a molecular electronics sensor that utilizes DNA hybridization as its primary sensing mechanism, in order to obtain benefits of testing speed, simplicity, robustness, and broad applicability for genetic analysis that hybridization-based detection can provide.

A molecular electronics sensor for genetic analysis is also disclosed and provided, including methods of use for this sensor. These embodiments have the benefits of providing for faster testing, lower cost testing, lower cost test apparatus, and testing that is simpler to perform, and that also enables highly distributed deployment or point-of-use deployment of such testing systems, including mobile use and home use of such testing systems.

An all-electronic, single molecule detector of DNA or RNA segments of interest and the concentration of such segments is also disclosed and provided. Certain embodiments include methods for these sensors to be deployed in a semiconductor chip format, and more desirably in a CMOS chip device format.

Another object of the invention is to provide methods to perform highly multiplexed measurements on such chip devices, in order to provide the benefits of low cost, rapid and portable testing, and the benefits that such chip-based devices, systems and kits can be manufactured at extremely high volume and low cost by leveraging the existing manufacturing base of the semiconductor industry. It is also the object of the invention to provide methods by which these disclosed devices and systems can be used to address genetic analysis problems of importance specifically in the areas of the diagnosis and treatment of disease.

In another aspect, DNA tag arrays and DNA tag reporter assays are disclosed and provided using the hybridization sensors and sensor array chips disclosed herein as a universal and preferred means of performing a broad range of multiplex biomarker assays. These tag arrays provide the benefits of having a common detection platform based on the well-established method of hybridization detection, for many diverse assays, both DNA, RNA or nucleic-acid based, as well as protein detection assays or other biochemical analyte assays. These provide the benefit of massively multiplex detection capability, allowing scalable and high levels of multiplexing of diverse analyte assays. These also provide the benefit of highly optimized, robust and uniform performance of the reporter detection. These tag arrays further provide the benefit of separation of the primary detection assay, which can be done under preferred or standard solution conditions phase to generate the reporter tags, from the reporter readout part of the assay, that is performed on the molecular electronics hybridization sensor array chip. The reporter tags further provide the advantage that they can be readily amplified, by standard PCR processes for copying DNA, to improved detection sensitivity, whereas the primary target of detection may otherwise not be readily amplifiable, such as for DNA targets that contain epigenetic marks, such as methylation, or detection targets that are not DNA, such as protein targets or other molecular targets.

It is also the object of the invention to disclose and provide methods that extend these benefits to the problems of genetic analysis that occur in the field of infectious disease, for detection of the pathogens or pathogenic strains that cause such diseases, including pathogens in the form of parasites, fungi, bacteria and viruses. In particular, this is a benefit for viral disease, such as influenza, colds/respiratory viruses, including rhinoviruses and adenoviruses, AIDS virus/HIV, Ebola, Dengue, other hemorrhagic fever viruses, Hanta, Zika and West Nile Virus, SARS, MERS, and novel viruses with pandemic potential, such as COVID-19, for which the benefits of low cost, broadly deployed, rapid testing have great value in preventing or controlling the potentially rapid spread of these disease which can have massive public health and economic impact.

It is also the object of the invention to extend these benefits to the domain of infectious diseases testing in the domain of sexually transmitted diseases (STDs), which are predominantly caused by pathogenic parasites, fungi, bacteria and viruses, and where it is a particular benefit to have detection systems that are well suited to widespread deployment for use in community clinics or in the privacy of the home, by virtue being low cost, rapid, simple to use, smart and connected electronic testing devices.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed.

FIG. 1 Shows a molecular electronic circuit with applied voltage and measured current.

FIG. 2 Shows a DNA hybridization and a molecular electronic circuit where FIG. 2A illustrates the general processes of single stranded DNA hybridization to form a double stranded duplex DNA. and FIG. 2B shows the general concept of engaging a DNA hybridization probe into a molecular electronics circuit.

FIG. 3 Shows hybridization events generating sensor signals in the measured current.

FIG. 4 Shows a process provided herein such that when exposed to a complex pool of DNA targets, the hybridization sensor will generate stronger signals from proper hybridization binding events, and weaker signals from incomplete or off-target interactions.

FIG. 5. Shows an embodiment of a single sensor bridge that comprises multiple ss DNA hybridization probes.

FIG. 6. Shows the architecture of a CMOS chips sensor array device and pixel that provides for chip-based deployment of multiple molecular electronic hybridization sensors.

FIG. 7 Shows a CMOS chips sensor array device where each measurement pixels provides for the monitoring of multiple molecular electronic sensor elements, allowing multiple sensors per pixel.

FIG. 8 Shows the concept of how molecular electronics hybridization sensors can be fully deployed in applications in pathogen detection and monitoring.

FIG. 9 Shows a specific sensor embodiment used in demonstration experiments of primer concentration effects.

FIG. 10 Shows an examples signal trace from one pixel of a 16 k sensor pixel chip on which the sensor embodiment of FIG. 9 is deployed

FIG. 11 Shows a closeup view of the signal trace in the Primer binding phase of the sensor data shown in FIG. 10, and a histogram of the measurement values that indicates the relative time spent in on and off states.

FIG. 12 Shows a signal trace from a sensor on a 16 k pixel chip, for an experiment in which the concentration of target primer is serially raised from 10 nM (nano-Molar) to 100 nM to 1000 nM.

FIG. 13 Shows close-up signals traces from the three concentration phases of the experiment of FIG. 12, along with histograms that indicate the relative time spent in on versus off states.

FIG. 14 Shows the measured fraction of time spent in the on/bound state (X icons), versus primer concentration, for the experiment of FIG. 12, illustrating the relationship between target concentration and fraction of time bound. Also shown is a fit to the expected exponential decay curve.

FIG. 15 Shows a particular sensor embodiment used in demonstration experiments on the impact of perfect matches versus mismatches in the primers.

FIG. 16 Shows the sequences of the hybridization probe used in experiments on primer length, location and mismatches, and of the various different length and target site primers used in these experiments.

FIG. 17 Shows an example sensor signal pixel trace from a 16 k pixel chip, from an experiment that serially exposed the sensor to different lengths of target primer.

FIG. 18 Shows close-ups of the signal from the data of FIG. 17, along with calculated values of primer length, melting point (Tm), duration of on events, and off-rate.

FIG. 19 Shows the perfect match primer for the hybridization probe, and various mismatch primers with from 1 to 7 mismatches to the hybridization probe.

FIG. 20 Shows close-up signal traces from perfect match, single mismatch and triple mismatch primers, and the related measured on-times and off-rates.

FIG. 21 Shows different example embodiments for linking decoding probe hybridization targets to the primary hybridization probe.

FIG. 22 Shows alternative embodiments for conjugating the hybridization probe into a molecular electronics sensor, representing diverse attachment options.

FIG. 23 Shows alternative embodiments for conjugating the hybridization probe into a molecular electronics sensor, by use of a complexing molecule.

FIG. 24 Shows the use of probe secondary structure in conjugation with a signal enhancing group in a molecular electronics hybridization sensor embodiment provided herein.

FIG. 25 Shows the use of probe hairpin secondary structure in combination with a signal enhancing group in a molecular electronics hybridization sensor embodiment.

FIG. 26 Shows the use of probe hairpin secondary structure, with mismatches, as well as a bridge oligo secondary structure, in combination with a signal enhancing group in another molecular electronics hybridization sensor embodiment.

FIG. 27 Shows the use of probe secondary structure with a mismatched protection strand, to create a parallel bridge structure in combination with a signal enhancing group, in another molecular electronics hybridization sensor embodiment.

FIG. 28 Shows the use of probe secondary structure with a mismatched protection strand, to create the primary bridge structure, in combination with a signal enhancing group, in a molecular electronics hybridization sensor embodiment which opens the circuit upon hybridization.

FIG. 29 Shows the use of probe secondary structure with a mismatched protection strand, to create the primary bridge structure in combination with a signal enhancing group, in another molecular electronics hybridization sensor embodiment.

FIG. 30 Shows the use of the probe as a primary bridge structure in a molecular electronics hybridization sensor, in which hybridization produced a double-stranded bridge.

FIG. 31 Shows the use of a hybridization probe in an electronics hybridization sensor, in which the target strand is labeled with a signal enhancing group.

FIG. 32 Shows a general overview of a DNA tag set, and their property of being well-behaved when applied as multiplex solution phase targets for their respective complementary specific hybridization probes on a sensor array.

FIG. 33 Shows an overview of an embodiment of method herein of a molecular electronics DNA Tag array for reporting and quantification of multiplex assay results.

FIG. 34 Illustrates a PCR-based assay with a DNA Tag reporter for detecting DNA targets in a complex pool.

FIG. 35 Illustrates an allele-specific PCR-based assay with a DNA Tag reporter for detecting DNA allele targets in a complex pool.

FIG. 36 Illustrates a Rolling Circle PCR-based assay with a DNA Tag reporter and using circularization probe, for detecting DNA targets in a complex pool.

FIG. 37 Illustrates a Rolling Circle PCR-based assay with a DNA Tag reporter and using an allele-specific circularization probe, for detecting DNA Allele targets in a complex pool.

FIG. 38 Illustrates a test ligand interaction assay with a DNA tag reporter, for detecting the interaction targets of a test ligand in a multiplex pool of candidate interacting ligands.

FIG. 39 Illustrates a ligand-ligand interaction assay with a DNA tag reporter, for detecting the interactions between a pool of candidate interacting ligands, and using a bipartite tag construct.

FIG. 40 Illustrates various preferred molecular methods for forming a bipartite DNA tag via ligation processes.

FIG. 41. Illustrates the binding of two DNA strands (A) An oligonucleotide 5′-TACGTGCAGGTGACAGG-FAM was attached to the polypeptide bridge using conventional click chemistry at its 5′ end. (The 3′ fluorescein was added to assist during purification and to prevent polymerase binding at the 3′-OH in other experiments.) (B) Shown is a 6-second current measurement made in the presence of 20 nM target 17-mer 5′-CCTGTCACCTGCACGTA in Buffer A. Each pulse of current above the 30 pA baseline represents a single binding event. The durations of the events and the pause between events occur stochastically and are described in the Supplementary Information. (C) The smoothed histogram at right suggests that the Probe is bound to Target approximately 22% of the time.

FIG. 42. Illustrates the binding a 14-mer DNA oligonucleotide to a 17-mer DNA (5′-TACGTGCAGGTGACAGG) attached to the Sensor bridge. A-C are 1-second-long plots of sensor current (pA) with 10 nM (A), 100 nM (B) or 1000 nM (C) 14-mer oligonucleotide target 5′-CCTGTCACCTGCAC that will bind the 17-mer. Baseline current on this pixel was between 15 and 20 pA in buffer A but when the target is added, current periodically increases to 25-40 pA. The width of the peaks remains constant (˜25 ms), but the time at baseline between peaks decreases from 45 to 4 ms as the concentration is increased, indicating faster binding at higher concentration. Fraction bound was estimated from the histograms (insets at right) of trace density. The graph D is of values estimated using HMM modeling for estimation of fraction bound and dwell time.

FIG. 43. Thermal melting of two oligonucleotides. For these experiments, a peptide bridge with a 45-mer oligonucleotide attached was used on the chip. Two target oligos of length 15 (CCTCTGTGAAGGCCT) and 20 (CCTCTGTGAAGGCCTGATCG) were added to the chip at a concentration of 20 nM. The sample chamber equilibrated at 41° C. using a thermoelectric device mounted under the chip. After equilibrating about 10 min., current readings were taken continuously while the temperature was increased in 2° C. steps up to 55° C. Temperature change was halted (to reduce noise) for 3-min reading intervals. Since the temperature was not calibrated in the sample solution, and the probe is attached to the chip surface, these values of Tm are not directly comparable to ones estimated by other methods. Curves were fit using the method of Petersheim M, and Turner DH.

FIG. 44. Shows the specificity of binding measurements and response to mismatched bases. For these experiments, a peptide bridge with a 45-mer oligonucleotide (5′-CGATCAGGCCTTCACAG AGGAAGTATCCTGTCGTIT AGCATACCC) attached was used on the chip. Targets (all 20-mers) were added sequentially, and binding kinetics monitored to determine fraction bound. Results are tabulated in the figure showing a distinct downward trend in both fraction bound and dwell time as the number of mismatched nucleotides is increased. These measurements were done using ˜4000 binding events each. The change in affinity of even a single mismatch (in 20) is readily detectable in either fraction bound or dwell time even when dwell time is as short as 4-5 ms.

FIG. 45. Shows a determination of binding kinetics between wild type and mutants. For a given same time scales the WT vs single nucleotide substitutes will show different binding kinetics with unique characteristics.

FIG. 46. Illustrates the known diversity region mapped in N-gene for COVID.

FIG. 47. Shows the specificity of binding measurements and response to different numbers of mismatched bases.

FIG. 48. Illustrates that SNPs in the COV N1 gene can be distinguished by binding kinetics.

FIG. 49. Shows the kinetics binding results from a full match for the N1-gene binding with a N1 gene on a bridge.

FIG. 50. Shows the kinetics binding results from a single nucleotide substitution for the N1-gene binding with a N1 gene on a bridge.

FIG. 51. Shows the kinetics binding results from a three-nucleotide substitution for the N1-gene binding with a N1 gene on a bridge.

FIG. 52. Shows the kinetics binding results from a four-nucleotide substitution for the N1-gene binding with a N1 gene on a bridge.

FIG. 53. Shows a kinetics binding results between a N1-match, a N1-triple, and a N1-Quad comparison.

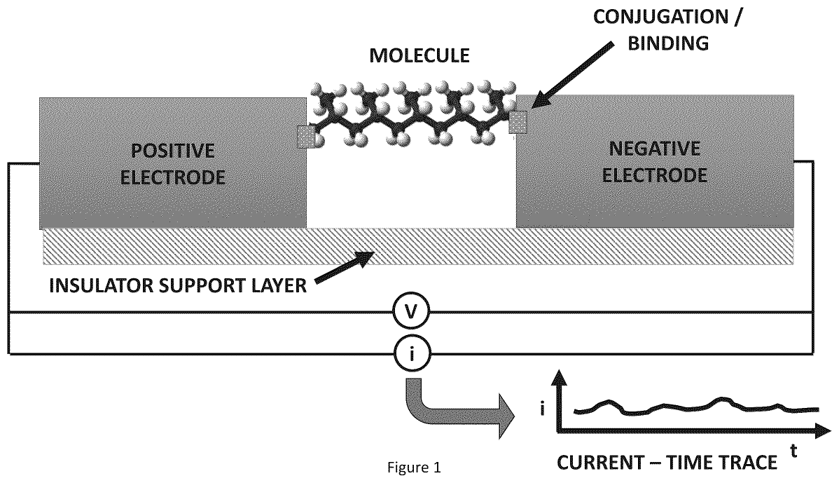

### DETAILED DESCRIPTION

### Definitions

As used herein, the term “DNA” refers generally to not only to the formal meaning of deoxyribonucleic acid, but also in contexts where it would makes sense, this term also encompasses the well-known nucleic acid analogs of DNA that are used throughout molecular biology and biotechnology, such as RNA, or RNA or DNA that comprises modifications such as bases with chemical modifications, such as addition of conjugation groups at the 5′ or 3′ termini or on internal bases, or which includes nucleic acids analogues, such as PNA or LNA. DNA may generally refer to double stranded or single stranded forms as well in contexts where this makes sense, and unless specifically designated. In particular, when referring to hybridization and the probes and targets for this as DNA, they are interpreted in this broader sense of any of these analogs which undergo hybridization to form a bound duplex.

As used herein, the term “hybridization” or “DNA hybridization” refers to the process by which a single stranded segment of DNA in solution pairs with its reverse complement sequence to form a duplex molecule via Watson-Crick base pairing, and forming a double helical segment. It is understood here this includes the cases of DNA-RNA pairs forming, RNA-RNA pairs forming, and that such DNA could also include modified bases or nucleic acid analogs such as PNA or LNA. It is understood this pairing can occur between single strands of different length, such pairing occurring between the complementary segments of these longer sequences.

As used herein, the terms “complement”, “match”, “exact match” and “reverse complement” of a given segment of single stranded DNA or RNA all refer another single strand of DNA or RNA that will hybridize properly with this strand to form a duplex with Watson-Crick base pairings (and base pairing U-A for RNA-DNA or RNA-RNA pairings, as RNA has Uracil (U) instead of Tymine (T)) for the segment of interest.

As used herein, the term “hybridization probe” refers to a specific segment of DNA (or RNA) that is to be used to bind a complementary strand of interest. Such a strand of interest may exist within a sample or complex pool of known or unknown DNA or RNA fragments, or a diverse set of oligos presented in a solution environment that allows for the hybridization reaction. This term also may refer to the segment that will be anchored in place for exposure to the test sample solution. In context, the hybridization probe may refer to the single molecule of interest, or to a quantity of such molecules that all have the same sequence. A hybridization probe in many instances may be a short segment of DNA, in the range of 10-100 bases, but in general can be a DNA strand of any length. As used herein, the hybridization probe may generally refer to a DNA segment for which only a portion of it is used to hybridize to a target of interest, and other portions of it may serve difference purposes, such as spacers, segments comprising conjugation sites, segments intended to hybridize to other distinct targets, segments intended to bind DNA primers, or sites for binding of decoding probes use to produce location maps for the sensor on a chip, including segments that are sites for hybridization to targets that are decoding probes that are DNA hybridization oligos, including such oligos used for combinatorial decoding, which oligos which may be otherwise labelled or unlabeled with additional signaling groups to aid in decoding.

As used herein, the term “primer” refers to a single stranded DNA oligo that has a hybridization binding site on a single stranded DNA template molecule of interest, and the term “primer binding” refers to hybridization of the oligo to its target site. This term arises from the well-known process of priming a single strand for synthesis of the complementary strand by a polymerase enzyme, however in the present context, primers and primer binding are merely an alternate way to refer to the process of an oligo DNA, that binds to its complementary site via hybridization, in a context where the primer is typically a relative short segment, 6-60 bases, and more commonly 12-40 bases, or 16-25 bases in length.

As used herein, the term “primer extension reaction” or “primer extension” refers to the reaction in which a polymerase enzyme binds to the free 3′ end of a primer that is hybridized onto a complementary template strand to form a duplex, and, provided with suitable dNTP substrates, then synthesizes a complementary strand of the template, extending the phosphate backbone of this strand from this initial 3′ end of the primer. Such a primer extension reaction may extend just a single base, or more commonly, it may extend multiple bases along the template. Such an extension process may go to the end of the template, or terminate before the end of the template is reached, depending on reaction conditions and properties of the enzyme.

As used herein, the term “decoding probe” generally refers to any molecule whose binding and subsequent detection is used for a process of determining the location map of where hybridization probes for different targets are located on a sensor pixel array. In this context, it is assumed there are a multiplicity of different types of DNA hybridization probes, having different target DNA as defined by the probe sequences, and that molecules of these types have been randomly assembled into a sensor pixel array, or otherwise placed in such a way that their location in the pixel array is unknown. It this context, each hybridization probe is assumed to have physically linked or connected to it, one or more binding sites that would bind to one or more of the decoding probe molecules. The series of decoded probes are applied to such an array in series or in pooled form, allowed to bind to their specific targets on the hybridization probes, and the bound state is read out using the detectable signal generated by the binding probes. Such binding probes in preferred embodiments are single stranded DNA oligo hybridization probes, with hybridization targets on or linker to the DNA hybridization probes on the array. In preferred embodiments, the detectable signal is the electrical hybridization signal measurable by the sensor. In other preferred embodiments, dye labels on such probes could be read out with an optical microscope imaging system. Other embodiments could use binding probes that are not based on DNA hybridization, such as using aptamers or antibodies or libraries of small molecules.

As used herein, the term “combinatorial decoding” generally refers to any process of decoding the location map of hybridization probes on an array, where a series of outcomes of multiple decoding probe binding reactions is used to generate a unique identifier “barcode” for the array probes, that determines the hybridization probe identity.

As used herein, the term “hybridization target” means DNA or RNA molecules which contain the complement of a DNA hybridization probe. Such a target could be the exact complementary strand, but in most cases, it will be a longer strand that contains a segment exactly complementary to the probe. In the context of discussing a target with mismatches, it refers to molecules which match to the probe except at one or more bases, as indicated. In the context of hybridization, “perfect match” means a sequence that correctly hybridizes to the probe, with no mispaired bases, while a “mismatch” refers to a sequence that may bind to the probe, but has one or more mispaired bases, i.e. bases not engaged in the standard Watson-Crick bond found in natural double helix DNA-DNA or double helix DNA-RNA pairings. Such incomplete pairing will have reduced stability compared to the perfect match binding, which can be generally be used to discriminate perfect matches from mismatched forms—also known as cross-hybridization—in assay methodologies.

As used herein, the “hybridization assay” means any assay or test that comprises the process of hybridization.

As used herein, the term “sample” or “biosample” refers to any material that is intended for testing. Such material could be in solid or liquid form, and may also generally be in some form of container, such as a tube, and/or reside a carrier medium such as a swab or filter paper. Such material could comprise tissue, cells, bodily fluids, excrement, food products, portions of plants, of materials collected by a swab, air filter or water filter. Such material may also be maintained with some form of preservative or stabilizing agents. These terms may refer to the material in the state as initially collected, or materials that have undergone process steps, such as to extract or amplify DNA or RNA, prior to being in a form suitable to introduce to the sensor device.

As used herein, a “DNA tag reporter assay” or “tag reporter assay” “tag assay”, refers to an assay in which DNA tags are produced by the assay reaction, and where the detection results of the assay are encoded by the presence of the corresponding tags, or by the abundance of such tags produced.

As used herein, a “DNA tag” refers to a single stranded DNA oligo that may be used as a reporter tag in a tag reporter assay. It is understood that in contexts where this makes sense, that the term “DNA tag” may further refer more generally to a larger DNA segment that contains the tag segment, the double-stranded DNA duplex form where the tag constitutes one strand, or the reverse complement of the tag which would hybridize to the tag to yield the duplex form. As used herein, the term “tag complement” refers to the reverse complement of the tag in question, or, in contexts where it makes sense, a larger strand comprising the reverse complement.

As used herein, the term “tag probe” refers to the hybridization probe that has as its specific target the tag in question. Thus, such a probe consists of or comprises the reverse complement of the tag. The term “tag sensor” refers to the hybridization sensor that comprises the tag probe.

As used herein, a “DNA tag array”, or “tag array”, refers to a molecular electronics hybridization sensor array in which the probes on the array correspond to the single-stranded DNA tag complements, or single stranded DNA tags, corresponding to a given set of DNA tags.

As used herein, a “DNA tag set” or “tag set”, refers to a specific set of DNA tags, and in preferred embodiments, such a set that has been designed and otherwise selected to perform well for readout with hybridization tag arrays, under a common hybridization reaction condition.

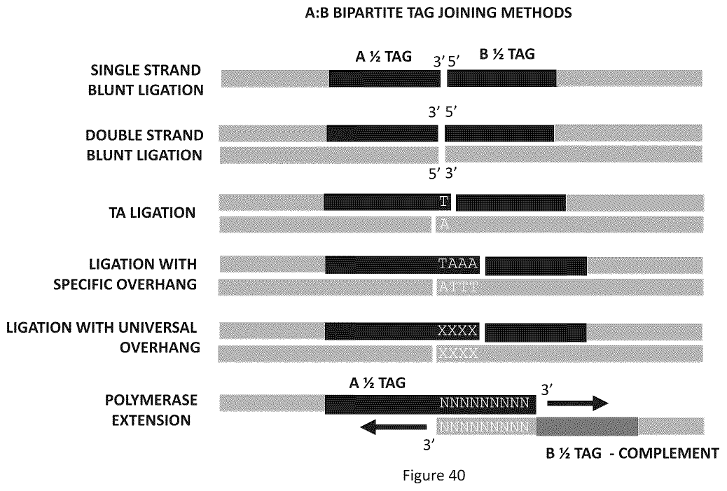

As used herein, the term “bipartite tag” refers to tags whose sequences are formed by joining together A and B sequences, possibly with joining sequence inserted, and wherein in actual assays, the corresponding physical tags are generated by physical joining of such partial tag sequences, by the methods as in FIG. 40. The term “bipartite tag set” refers to the total set of N=M×M such tags that comes from all possible joins between M A/12 tags and M B ½ tags.

As used herein, the term “PCR” refers broadly to any methods that use polymerase or reverse-transcriptase reactions to produce multiple copies of sequences from source DNA or RNA. In this context, the term “copies” may in general refer to single stranded reverse complements of segments of the source molecule, or single stranded exact copies of segments of the source molecule, or double stranded forms where one strand is identical to a segment of the source molecule. The term “copies” also may refer to the product of methods where an RNA template is converted to DNA molecules of the corresponding sequence, or a DNA template is converted to RNA molecules of the corresponding sequence. Such “PCR” methods in this context may include methods with linear amplification or exponential amplification, relative to time or cycle numbers. Such methods include those that use specific primers, or degenerate primers. Such methods also include isothermal reactions that occur in continuous time, or reactions that rely on thermal or chemical cycling. The “PCR” process may produce copies of specific target segments of the source DNA or RNA, as defined by specific primers, or may produce copies from many sites or random sites, as may result from degenerate primers. In particular, “PCR” in this connect may refer to isotheral amplification methods that can be used to rapidly produce large amounts of DNA copy fragments from a source genome, of RNA or DNA, using one of the many well-known methods, such as Rolling Circle or RCA, Genomify, or LAMP, and with such a method incorporating a reverse-transcriptase in the case of RNA starting material.

As used herein, “amplification” of DNA or RNA in a sample material refers to the use of PCR methods such as above, to make copies of the source DNA or RNA.

As used herein, “pathogen” refers to any disease-causing agent that has a genome, such as parasites, fungi, viruses, or bacteria, or other single or multicellular organisms that cause disease.

As used herein, “strain” refers the genetic variants within a species, i.e. members of the same species that have genomes that difference in sequence.

As used herein, “molecular electronics” refers to devices in which a single molecular or single molecular complex is integrated into an electronic circuit.

As used herein, a “molecular electronics sensor” is a device that transduces molecular interactions with target molecules in solution into electronic signals, using a single molecule or molecular complex integrated into an electrical circuit as the primary transduction mechanism.

As used herein, a “molecular complex” refers to small number of molecules that are held together by chemical conjugation, bioconjugation, or covalent or non-covalent bonds, such that the assembly is expected to retain this configuration or affiliation during the process of assembling it onto nanoelectrodes, and during use of the resulting sensor in assays. Such small number of molecules may be just two, such as a DNA oligo probe bound to a bridge, but in other contexts may be in the range of 2-10, 10-100, or 100-1000.

As used herein, “nanoelectrodes” are conducting elements that define a nanometer scale gap, and have dimensions of nanometer scale height and width, and substantially longer length, which provide an electrical conducting connection into a circuit.

As used herein, “bridge” or “bridge molecule” refers to any type of molecular wire or conducting molecule than may be using to make a conducting connection across the gap between nanoelectrodes. Such molecules include biopolymers, double stranded DNA, peptide or protein alpha helices, graphene nanoribbons, pilin filaments or bacterial nanowires, other multichain proteins or conjugates of multiple single-chain proteins, antibodies, Carbon nanotubes, or conducting polymers such as PDOT. Such molecules may include attachment groups that provide for specific attachment to, and/or self-assembly to, the nanoelectrode contacts.

As used herein, “semiconductor chip” refers to an integrated circuit chip comprising semiconductor materials such as Silicon or Gallium, and fabricated with techniques from the semiconductor industry.

As used herein, “CMOS chip” refers to an integrated circuit chip, fabricated using CMOS process techniques from the semiconductor industry. CMOS is an acronym for Complementary Metal-Oxide Semiconductor, and refers to a specific manufacturing process for making integrated circuit chips of the type most produced for processors, DRAM memory, and digital imager devices. As used herein, “CMOS chip” also refers to a device fabricated at the foundries that make such chips in industry, but which may also be postprocessed for purposes of the present disclosure if, using processes to adding or exposing accessible nanoelectrodes, an suitably protecting such nanoelectrodes, for use in the molecule electronics sensors.

As used herein, the term “chip” used in isolation refers to a “semiconductor chip” or “CMOS chip”.

As used herein, the term “pixel” refers to a sensor and measurement circuit that is repeated throughout a regular rectangular array of such identical circuits on a chip. A pixel may in context refer to just the measurement circuit, which here is a form of current meter measuring circuit, or may also include the sensor transducer element or elements affiliated with the circuit, which here are the molecular electronic component, i.e. molecule attached to nanoelectrodes. For definiteness, the term “measurement pixel” as used herein refers to the measurement circuitry of the pixel, and the term “sensor pixel” refers to the pixel circuit affiliated with a given sensor element. The origins of this term come from image sensors, where such pixels contained light sensing elements and measurement circuitry, which captured an element of a picture, but in the present context, as used herein the term pixel is unrelated to light sensing or imaging, and the pixels disclosed herein are sensing chemical interactions, not light.

As used herein, the term “sensor” refers to the complex consisting of the nanoelectrodes, bridge and hybridization probe, which is the primary transducer of interactions of the hybridization probes to electrical signals. In contexts where it makes sense, sensor could also refer to this plus the supporting current measurement circuitry, such as the including the pixel circuits. “Sensor pixel” refers to the pixel circuitry that provides measurements to a particular sensor.

As used herein, the term “signal group” or “signal enhancing group” refers to a chemical group that could be added to an oligo, and such that the presence of this group complexed into the probe-bridge complex, versus dissociation from this complex, produces a detectable signal. In particular, such a group may be displaced from the critical position by target probe binding, or may be brought into proximity as a label on the target strand.

As used herein, the term “secondary structure” refers to the physical conformation that a DNA strand takes in response to bonds it forms with itself or other molecules. In particular, this includes the structures that form from hybridization between portions of a DNA molecule, or between two DNA molecules. This also includes structure that may result from the DNA strand interacting with the bridge. Secondary structure can be induced by hybridization binding, and other forms of binding.

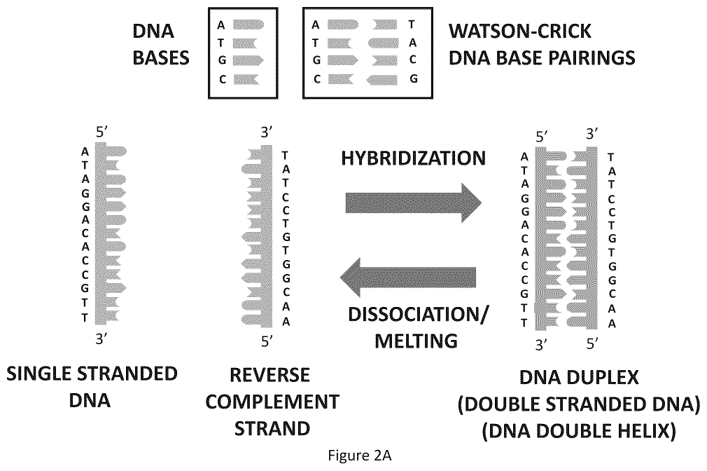

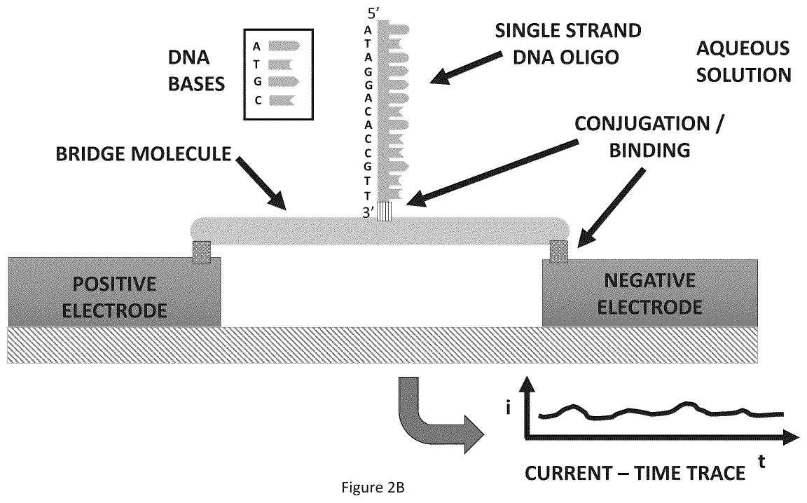

In various aspects of this disclosure, a DNA hybridization probe, which is a short piece of single stranded DNA, is attached by various means of conjugation to a bridge molecule that itself spans between two nano-electrodes and is suitably attached to each on either end, by some means of conjugation or binding. This configuration is further established within an aqueous solution. One preferred embodiment of this configuration is illustrated in FIG. 2B. Voltage is applied across the electrodes, which is accompanied by current flow through the bridge molecule. This current can be measured versus time, and this forms the primary measurement output of the device, as indicated in FIG. 2B Either end of the probe DNA (5′ or 3′) could in general be the end attached to the bridge, the FIG. 2B merely shows one of these embodiments, and is not limiting. In preferred embodiments, an internal base in the probe, rather than either ends, may also be the site for the conjugation. The DNA probe shown in FIG. 2B is a linear strand, but this is not meant to be limiting, and in other preferred embodiments it could be formed into a closed loop, have both ends attached to bridge, or comprise multiple linear segments joined by linkers, and in general need only be a molecular construct or complex that comprises a single stranded segment having the hybridization probe sequence of interest. Nor is it essential that the entirety of this segment engage in the specific hybridization of interest, in preferred embodiments it may be only a subsegment that engages in the hybridizing the target DNA. Nor is the length shown there (13 bases) meant to be limiting, the length could be in the range of 4-1000 bases, preferably 10-100 bases, and more preferably 12 to 50 bases, and in general the appropriate length range among these depends on the application. The gap spacing suggested by FIG. 2B is on the order of 10 nm, which is also not limiting, in various preferred embodiments, this gap could be in the range of 3-10 nm, 10-30 nm, 30-100 nm, or 100-1000 nm, or 1000 nm-10,000 nm. In various preferred embodiments, magnitude of applied differential voltage across the electrodes may be in the range of 0.01 Volts to 10 Volts, 0.05V to 3V, or 0.1V to 1.2V. In various preferred embodiments, this may be a DC voltage, or an AC or time varying voltage, either sinusoidal, periodic or varying according to some other applied waveform. In various preferred embodiments, there may also be a reference and working electrode provided to the solution phase, the set and control the potential of the solution relative to the potential of either electrode or to the ground of the circuit. Such electrodes may be platinum electrodes (or pseudo-electrodes as these are sometimes called), Silver/Silver Chloride electrodes, or other types of electrodes use to set solution potential known to those skilled in the art of electrochemistry. The resulting current measured in various preferred embodiments, may have baseline or typical magnitudes in the range of 1 pico-Amp (pA) to 10 pA, or 10 pA to 100 pA, or 100 pA to 1000 pA=1 nano-Amp (nA), or 1 nA to 10 nA, or 10 nA to 100 nA. In various preferred embodiments, there may also be a buried “back gate” electrode some distance underneath the insulating substrate, that may be used to apply an additional modulating voltage or field to the vicinity of the bridge and hybridization probe. In preferred embodiments, such an electrode may be buried 1 nm-10 nm below the surface, 10 nm-100 nm below, or 100 nm-1000 nm below. In preferred embodiments, such an electrode may also have lateral localization such that it is localize to be near the footprint of the bridge, in one or both lateral dimensions, such as extending no more than 10 nm or no more than 50 nm, or no more that 500 nm or no more than 1000 nm beyond the footprint of the bride, in one or both lateral dimensions.

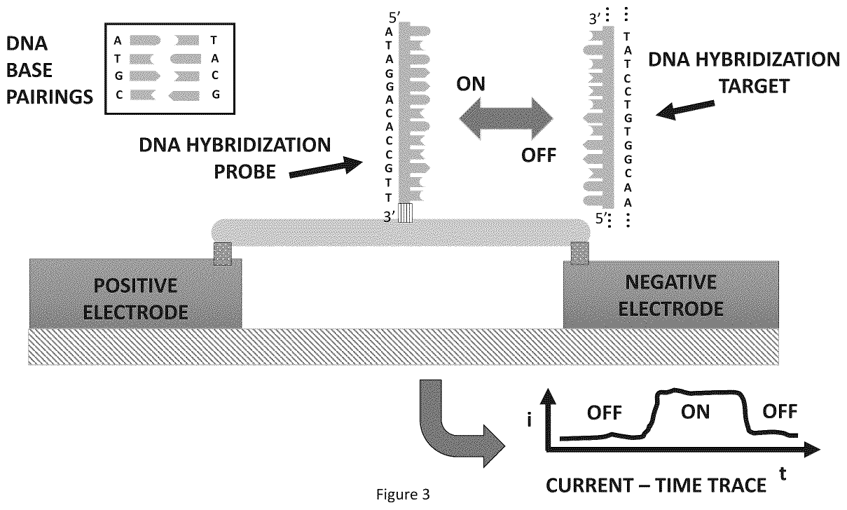

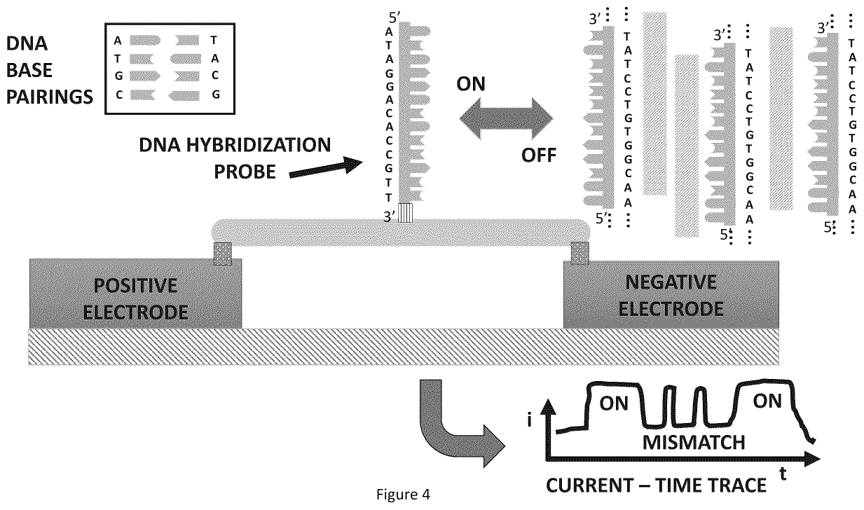

It is an object of the invention that this disclosed composition can function as a molecular electronics sensor for hybridization events. When such a molecular electronic sensor is exposed to a solution that contains single stranded DNA material, if the hybridization probe encounters and binds by hybridization to its intended exact match target, a distinguishable signal is produced in the measured current. This is illustrated in FIG. 3, which shows the probe encountering a segment (of a longer DNA molecule as indicated) that is an exact match (i.e. reverse complement sequence), and the current changing—there shown as jumping up to a larger value-when the probe has hybridized to this target, indicated as the “on” state, and then the current reverting back when the probe dissociates to the “off” state again. This signal thereby provides the primary detection of the target being present in the sample. If the exact match target is not encountered, there is no such distinguishable signal. Furthermore, as indicated in Figure. 4, when the this sensor is exposed to complex pool of DNA fragments, possibly including hybridization targets, indicated by the two specific complementary sequences shown, but also containing many non-matching or off-target fragments, indicated by the shaded anonymous segments shown, the hybridization events with exact matches produce the long duration detectible signals, which the many interactions with non-matching segments may result in no detectable jump in current, or many result in transient, rapid or shorter duration pulses that are readily discriminated for the stronger (i.e. longer duration) correct match detection pulse or step. This different in signal between the exact match hybridization event, and diverse non-specific interactions with other fragments, include interactions with just one or a few mismatches, provides for the primary detection of targets of interest in a sample, relative to a background of many other off-target fragments, even if such fragments may differ by only a single base from the target. Thus, this provides a molecular electronic single molecule hybridization sensor that can detect the presence/absence of a target DNA of interest in a potentially complex pool of DNA as might be extracted from a biosample of interest by common sample preparation methods for extracting, purifying, or amplifying DNA (or RNA). As shown in FIG. 4, it is suggested that the target one events result in a long step-up pulse, while the off-target events result in short step ups of similar magnitudes but shorter duration. These details of the Figure are not meant to be limiting, and in preferred embodiments, the exact target binding events might result in a step up or step down in magnitude of current, and this might be a step that does not return to baseline in conditions where the target stays bound. The off-target events in preferred embodiments may also make pulses of shorter duration and/or smaller amplitude, or that may otherwise have a signature in the current trace that differs from the exact match signatures. In preferred embodiments, the conditions of the operating temperature, buffer chemical composition, pH, applied voltage, back gate voltage (if any), solution potential, or other parameters of the system may be modified to enhance or optimize the ability to discriminate between exact match hybridization signals and various background interaction signals that the probe may engage in, or that are noise arising from other aspects of the molecular electronic system. In terms of buffer composition, there are many factors that may be used and that are known to those skilled in molecular biology that may impact hybridization reactions, such as concentrations of salts (such as NaCl, KCl), divalent cations (such as Mg++, Mn++, Ca++, Sr++), detergents and solvents (such as Triton or DMSO or betaine), and the primary buffering agent, such as Tris, HEPES or MOPS.

In some preferred embodiments, the nature of the detectible signal produced by the presence of the target is a series of spikes in the current, that correspond to target DNA binding to the probe, and then coming off of the probe. This is in general an expected behavior, as the hybridization binding is reversible, and the rate of binding “on” is influenced by concentration of the target DNA in solution, as well as composition of the buffer, pH, temperature, etc., as is the rate of coming “off” for a bound target also dependent on temperature, and such properties of the solution.

In some preferred embodiments, the properties of the observed spikes, such as the length of time from exposure to first observation an exact-match step up, the time between pulses, or the ratio of time on to time off, or other properties of the on/off rates, are relatable to the concentration of the target, and therefore provide a measure of concentration of the target of interest. Thus, by analyzing the data to extract and compute such measures, this provides a molecular electronic single molecule hybridization sensor that can detect concentration of a target DNA of interest in a sample, including samples with a background that may be a complex pool of off-target fragments.

In some preferred embodiments, a perfect match hybridization between the probe and target DNA will produce a detectible signal, and a single based mismatch in an off-target DNA relative to the probe DNA in the sensor will produce a distinguishably altered signal, and further levels of mismatches will produce even more distinguishably diminished signals, or little or no detectible signals. In this way, the sensor signal can be used to distinguish targets that are a perfect match to the hybridization probe DNA from other fragments that have even a single base mismatch. This provides sufficient sensitivity and selectivity to perform the genetic analysis applications of genotyping and strain determination, which often require the ability to discriminate DNA targets that differ by as little as a single base mismatch, or otherwise may differ by just a few bases of mismatch, or by insertions or deletions of one or a few bases.

In particular, the identification of Single Nucleotide Polymorphism (SNP) genotypes becomes possible, as these require single base discrimination among different DNA segments. For this, we disclose a method for SNP genotyping, where in the organism or biosample of interest, two or more sequence variants may be present, differing between any two by as little as one base substitution, or one base insertion or deletion, and specific hybridization probes are made for each such sequence, and put into molecular electronic sensors of the type disclosed. From a primary bio-sample of interest, DNA or RNA is suitably purified, using any of various means well known to those skilled in molecular biology, such as using an extraction column or phenol-chloroform extraction, and the purified sample is applied to these sensors, either separately, in different reaction volumes, or within one reaction volume applied to a device containing all such sensors. Such a device could be a CMOS chip with all such sensors present on the chip. By monitoring the signal results from each sensor, it can then be determined which if any of the variant targets are present in the sample, and this information can be used to determine a genotype for subsequent interpretation, or to identify the presence of one or more specific strains of a pathogen. In preferred embodiments of this method, this could be determining the strain of a parasite, fungi, bacteria or virus.

Referring again to the general hybridization sensor disclosed, and as illustrated in FIG. 2.2, In preferred embodiments, the bridge may be any molecule that can serve as a conducting connection between the electrodes. Such a conducting bridge could, in preferred embodiments, be a double stranded DNA segment, an alpha-helical protein, a carbon nanotube, a graphene nanoribbon, a multi-chain protein such as a bacterial pilin filament or bacterial nanowire, or a conducting polymer such as PDOT. In preferred embodiments, the bridge is fabricated by bottom-up chemical synthesis, and is made to have a defined chemical structure, and to have engineered in specific binding groups at precise locations in the chemical structure to provide the conjugation or binding sites for the hybridization probe and the nanoelectrodes. In preferred embodiments, such a bridge molecule has a specific conjugation site to which the hybridization probe DNA is conjugated to, either covalently or non-covalently, through any of many possible conjugation mechanisms known to those skilled in the art of bioconjugation. In preferred embodiments, this conjugation could be a click chemistry coupling, such as DBCO-azide or TCO-azide or other copper or non-copper click reactions, an APN-thiol coupling, an amine-NHS ester coupling, a biotin-avidin coupling, a peptide-tag based coupling such as Spy-Catcher, or an AviTag or an Aldehyde Tag. In preferred embodiments, the electrodes provide exposed metal contact targets for binding the bridge, and in preferred embodiment, the metals may be from among gold, platinum, palladium, or ruthenium. In preferred embodiments, the bridge may be conjugated to the electrodes by a connection comprising a thiol-metal bond, dithiol-metal bond, amine-metal bond, carbene-metal bond, or diazonium-metal bond, or other carbon-metal bonds, or may comprise a metal binding peptide, of which many are known to those skilled in biochemistry, such as the known Gold Binding Peptide (GBP) with amino acid sequence MHGKTQATSGTIQS, and which many also be repeated in tandem 2-6 times, separated by short GS rich linkers, or alternatively the known Palladium binding peptide QQSWPIS. The conjugation could also be achieved by applying a bifunction linker with any of these or other binding groups use to attach one end to the electrode, and have a short linker such as a PEG or carbon chain 1-3 nm long, presenting arbitrary second conjugation group as the head group, such as a click chemistry group, that can then be used to conjugated to a cognate group on the bridge molecule.

In preferred embodiments, the DNA hybridization probe may be between 4 and 200 bases in length, preferably between 10 and 100 bases in length, and preferably 12 to 60 bases in length. In applications requiring single base discrimination, such as SNP genotyping or pathogen strain determination, the probe length is preferably 10 to 35 bases in length, or more preferably 15 to 25 bases in length. The probe may also comprise other nucleic acids or nucleic acid analogs, such as RNA, PNA or LNA, which may provide stronger binding or great specificity of binding. Such probes can have reduced length. The probe DNA may also comprise a fluorescent group, such as a FAM dye molecule on the 5′ end, and groups can be used for quality control or characterization in the synthesis and purification of the probe-bridge conjugates, or the characterization of the assembled sensors, such as in optically assessing whether a nanoelectrode has received a probe-bridge complex.

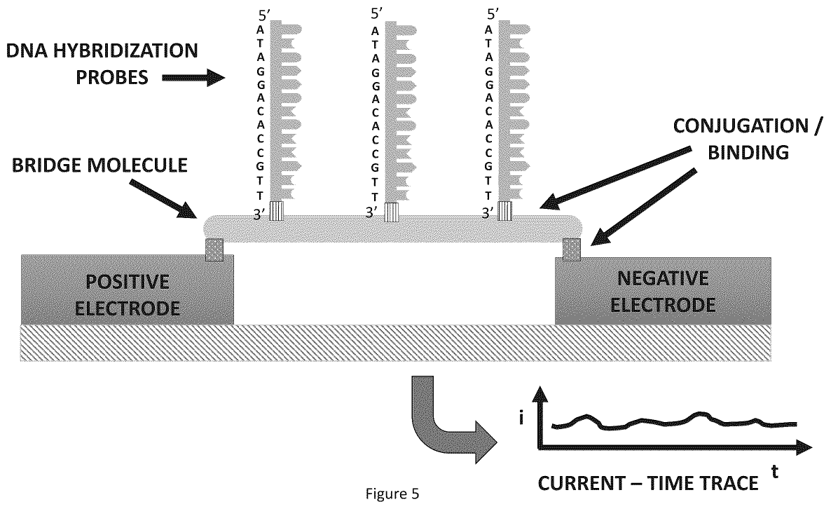

In preferred embodiments, the molecular bridge illustrated in FIG. 2.2 could have a length of 3-10 nm, or 10-100 nm, or 100-1000 nm, or 1000 nm-10000 nm. In preferred embodiments, as illustrated in FIG. 5, there could be more than one hybridization probe conjugated at specific sites on the molecular wire, as illustrated in FIG. 5. In preferred embodiment, such multiple probes may identical DNA probes, or distinct DNA probes, e.g. probes that target the same sequence, or different sequences. The benefits of multiple probes could include increased total signal, increased signal to noise, robustness against failures of probe conjugation, robustness against probes being in inaccessible configurations, or the ability to multiplex one sensor to detect multiple targets, for greater robustness or breadth of detection. Such multiple probes in referred embodiments would number between 2 and 10, between 10 and 100, or between 100 and 1000. Such probes may be located so as to point in similar orientations relative to the bridge, as suggested by the cartoon in FIG. 5, or they may be located so as to point in different orientations away from the bridge. This may provide a benefit of ensuring some probes are spatially accessible once the bridge is in place. The spacing between such probes along the molecular bridge would in some embodiments be in the range of 1-10 nm, or in the range of 10 nm-10 Onm. In preferred embodiments, the bridge molecule is fabricated by bottoms up synthesis to have these multiple probe conjugation sites at precisely defined locations in the chemical structure of the bridge.

In preferred embodiments, the application could be gene expression analysis of any cellular samples, and in general could be any application where methods such as DNA microarrays have been used for gene expression. In preferred embodiments, this would include gene expression applied to tumor tissue as may be used in cancer diagnostics.

In preferred embodiments, the application could be SNP genotyping in human, animal, or other cellular samples, and in general any application where methods such as DNA microarrays have been used for genotyping. In preferred embodiments, this would be applied to SNP genotyping in humans.

In preferred embodiments, the application is massively multiplexed hybridization probe detection and/or concentration measurement of targets in a complex pool. The level of multiplexing could be up to 100 different probes, 1000 probes, 10,000 probes, 100,000 probes, 1 million probes, 10 million probes, 100 million probes, or 1 billion probes. The provides an alternative to DNA microarrays for such high levels of multiplex detection, with the advantages of all-electronic, chip-based system, single molecule sensitivity, speed, low-cost consumables and instrument, and compact mobile, portable, or point-of-use instruments.

In preferred embodiments, the application could be species identification, in particular determining what species a given tissue sample is taken from, or in the identification of which pathogens, such as bacteria or viruses are present in a given sample. In preferred embodiments, this could be testing of environmental samples for the presence of a given virus, such as COVID-19. In a preferred embodiment, the sample could be a tissue, or fluid, from any of the common vectors for viral transmission, such as bats, birds, rodents or mosquitoes. In preferred embodiment the sample could be material filtered from air or water, or material swabbed from a surface. In preferred embodiments the sample could be a biosample taken from a human or animal subject, such as saliva, mucous, buccal swab, blood, sweat, urine, stool, or exhaled air.

In preferred embodiments, the application could be strain identification, in particular determining what strain of a pathogen, such as a bacteria or virus, are present in a given sample. In preferred embodiments, this could be testing of environmental samples for the presence of a given strain of a virus, such as COVID-19. The samples for this, could be the same as for the previous species identification application.

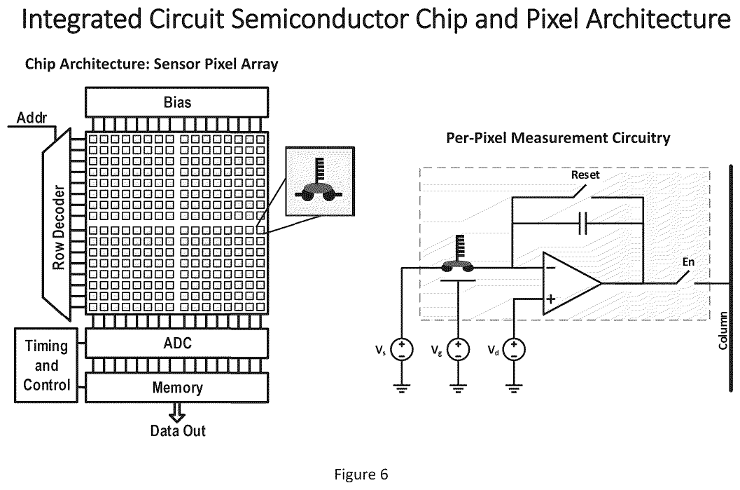

In preferred embodiments, these molecular electronics hybridization sensors are deployed on integrated circuit semiconductor chip devices, where such chips include the circuitry to supply voltages to the sensors, measure currents in the sensors, and transfer such data off-chip, and to control such operations. In preferred embodiments, and as illustrated in FIG. 6, the architecture of such a chip is as an array of pixel, with such pixels providing the circuits needed to apply voltages and measure the currents. In the preferred embodiment shown in FIG. 6, the chip has a rectangular array of pixels circuits, each such pixel comprising a molecular electronics sensor which transduces molecular interaction events into the current signal, indicated schematically by the cartoon inset of the hybridization sensor, as well as having other major architectural blocks as labeled, that apply the needed voltages (Bias), manage the transfer of measurements from the array, and their conversion to digital form, (Row Decoder and ADC, or Analog-to-Digital Convertor), and where in particular the ADC converts each analog measured value to a binary digital value, with a bit resolution that in preferred embodiments may be 8 bits, 10 bits, 12 bits, or 16 bits, or bit a depth selected in the range of 1-32 bits. In addition, other blocks indicated is a local on chip memory buffer (Memory), and the control circuitry (Timing and Control), which in preferred embodiments comprises circuits to produce, synchronize and distribute clock signals, including PLL circuits. One preferred embodiment of the pixel schematic circuit design is also indicated in FIG. 6, which here is a TIA circuit or Trans-Impedance-Amplifier. This circuit schematic shown allows indicates the application of Gate, Source and Drain Voltages, and the measurement of the resulting current by collection of charge onto a capacitor, which may be reset to an uncharged state or nominal state by closing the indicated “Reset” switch. Closing the “En” switch shown then outputs the measure data, i.e. The voltage across the capacitor, from the capacitor to the column bus and encoder.

These circuit blocks indicated in the schematic, as well as the blocks of the pixel, or the pixel circuit itself, can be fulfilled by many possible detailed circuit designs and IC layouts, well-known to those skilled in the art of VLSI Integrated Circuit Design, Digital Circuit Design, Mixed-Signal Circuit Design, and Analog Circuit Design. The architectures and schematics shown in FIG. 6 are not meant to be limiting on the use of such circuit designs or layouts that provide similar functionality.

In preferred embodiments, the molecular electronic sensor is deployed onto a CMOS chip device, which is a specific form of semiconductor chip and chip manufacturing process. The advantage of using CMOS chips is the very large manufacturing base for such chips, and related supply chains, as well as the aggressive scaling roadmap for such devices. The majority of chips presently made are of the CMOS type, including the common processors, memory, and digital imaging chips used in commercial products. Another advantage is that aggressive scaling has led to shrink the features on such chips down to the near the 1 nm scale, so that such processes are in principle capable of producing nano-electrodes needed for the present disclosed sensor, thereby enhancing the manufacturability of the devices disclosed herein.

In preferred embodiments, such a chip operates synchronously, by each pixel acquiring a single current measurement value, and then the array of such values are transferred off chip as a “frame” of digital data, in a row-by-row fashion as indicated in FIG. 6, at some total frame rate, which in preferred embodiments could be up to 10 frames per second, or up to 100 frames per second, or up to 1000 frames per second, or up to 10,000 or 100,000 frames per second. In preferred embodiments, the pixel array may contain up to 100 pixels, 1000 pixels, 10,000 pixels, 100,000 pixels, 1,000,000 pixels, 100,000,000 pixels, or up to 1 billion such pixels. The physical size of such a chip is related to the number of pixels, and in preferred embodiments may be as small as 1 square millimeter, and as large as a full reticle size used in for the photolithography, which may be up to approximately 30×30 millimeters squared.

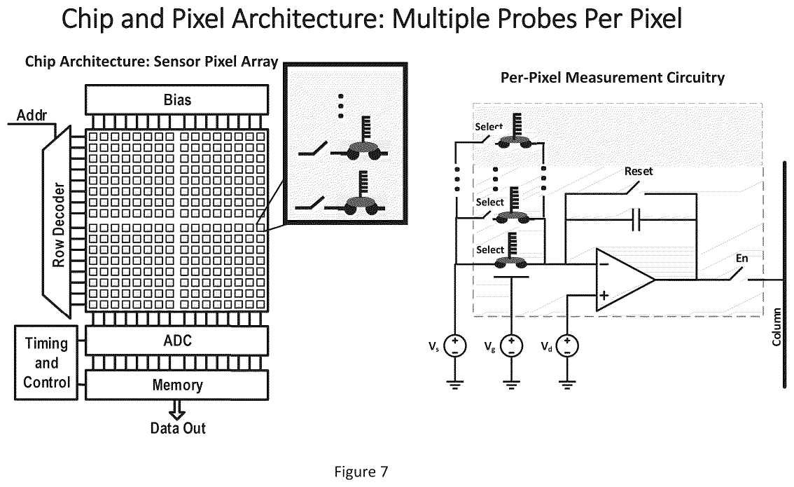

In such sensor array chips, in some preferred embodiments, as illustrated in FIG. 7, there are more than one pair of nanoelectrodes per measurement pixel, and therefore more than one molecular electronic sensor per pixel, so that one measurement pixel circuit may be used to monitor signals from multiple sensor devices (nanoelectrode pairs with bridge and hybridization probe molecule). In preferred embodiments of this, the measurement circuitry for applying voltages, measuring current, and reading out data in each pixel is applied serially in time to each sensor within the pixel, under control of transistor-based switches in the sensor circuit that can select any of the sensors for measurement. In FIG. 7, the addition of these selection switches is indicated by the switches labeled “Select” in the pixel schematic shown. By closing a single one of the available switches, the measurement circuit in the pixel is coupled in to just one sensor, and can be used to acquire data for that sensor. Such data can be transferred of the pixel in a frame transfer process, and then the pixel can be cycled on to the next bridge. In preferred embodiments this is done synchronously, so that all pixels acquire data with their local switch 1 closed, transfer a frame off the array, and hen all pixels move on to closing switch 2, etc. Cycling through all such switches in this way, frame by frame, if each pixel serves M sensors, a chip with N pixel circuits embodied on chip can acquire measurements from a larger (and potentially much larger) number, NM, of sensors. This in general has the potential to lower chip costs: the price of chips is based on area, and the area required for the chip is dominated by the area needed for pixel circuits, and this has the benefit of greatly lower the total pixel count and pixel circuit area required to serve the number of sensors of interest, versus having one pixel per sensor. In preferred embodiments, each pixel may have from 2-1024 sensors per pixel, and preferable in the range of 4-128 sensors per pixel. However, this form of multiplexing of sensors per pixel has the cost that measuring from all pixels takes longer, by the multiplier M, as it requires M times more frames, and also the disadvantage each sensor is offline and not measuring while its switch is open, so that the respective current goes un-observed for part of the time, creating the potential to miss signal features.

In preferred embodiments, the chip pixel array architecture is such that nearby pixels in the array share a common staging area, where the many nanoelectrode pairs for these neighbors are all located, and suitably electrically routed back to these adjacent pixel circuits. This applies to both cases where each pixel has one sensor affiliated with it, or cases of M multiple sensors per pixel. The use of such staging areas further improves the efficiency of circuit layout, and allows the staging area to have a larger opening, and better accessibility for nanofabrication or molecular assembly purposes or to facilitate wetting by the solution.

For arrays of sensor on chip, in one preferred embodiment multiple probes with the same target are represented on all or part of the array. In preferred embodiments, these multiple measures of the same target can be aggregated or averaged together to produce a more robust detection of presence/absence of the target, or to provide more sensitive detection or lower detection limits, or to provide a measure of concentration. For example, in preferred embodiments, if N sensors have the same hybridization probe and target, and a fraction f of these register a detection event, within a measurement time T of exposure to the sample, then detection becomes more robust or sensitive or accurate if a minimum threshold, fimin, is required for detection, f>fmin. Or, in other preferred embodiments, the ratio of f/T, which is the rate of detection, provides a measure of the concentration of the target in the sample. In other preferred embodiments, more detailed analysis of the f(t) curve acquired during the time interval [0,T] could provide various robust fits to the slope of this curve, or this curve could be fit to characteristic profiles or measured calibration curves produced by known reference concentrations, to provide a measure of concentration from these multiple probe measurements on the chip array. Such aggregation measures also typically provide a related estimate of confidence or measurement uncertainty, such as a suitable mean and standard deviation. If such individual probes are otherwise directly providing concentration measures, for each probe, these can also be averaged together, by various well-known means of averaging measurements, to produce a more accurate estimate of concentration, as well as error bars or confidence intervals on the measurement, based on the spreads or standard deviations observing in the set of individual measurements. This provides benefits of greater accuracy and measurement confidence for the concentration estimate for the target of interest.

For arrays of sensor on chip, in another preferred embodiment different hybridization probes with different targets are represented on some or all of the array. In preferred embodiments, these multiple measures of different targets provide the ability for multiplex or in-parallel measurements of the set of targets of interest. This has the advantage of lower cost of testing the targets, and faster testing of the targets, or simpler testing of the targets, or the use of less sample material or less reagents, to test for all the targets, versus separately testing for such targets on separate devices. This is generally referred to as multiplex testing or parallel testing, and is widely appreciated as a potential benefit to testing systems.

In preferred embodiments of sensor array devices, both forms of multiple probes will be present, i.e. for the give set of hybridization probes with the respective targets of interest, each specific type of hybridization probe will be represented by multiple, replicated sensors on the array, proving the benefits of redundant, replicate measurement above, and the multiplex probes for the multiple targets will be represented on the array, to also proved the benefits of multiplexing. The resulting compound benefit is multiplex testing, with confident measurements for each target that have the benefits of statistical replication for accuracy and confidence interval estimation. For this purpose, it a benefit in preferred embodiments to allow for very large chip-based large arrays of probes, in preferred embodiments up to 100 probes, up to 1000 probes, up to up to 10,000 probes, up to 100,000 probes, up to 1 million probes, up to 10 million probes, up to 100 million probes, or up to 1 billion probes, or up to 10 billion probes.

Multiplex Probe Maps and Decoding Methods In such preferred embodiments of sensor arrays in which multiple distinct hybridization probe sensors are deployed on one chip for the purpose of multiplex testing, there is a map provided that specifies what probe type is present at each different pixel location or sensor location (if multiple sensors are affiliate with each pixel). This allows the measured sensor data readout from the pixel array to be related to which probe target was being assessed at each sensor. Such a map may be produced by various techniques, based on how the probe molecules are prepared and applied to assemble into the array. This map is referred to as the probe map for the sensor array or pixel array.

In one preferred embodiment for establishing this map, spatially controlled exposure of the pixels to the different solutions containing the different probe types for assembly (or, instead of just the probe molecule, in preferred embodiments it is the probe-bridge molecular complex pre-formed which is assembled into the sensors) during sensor assembly, so that the probe map is known from which solutions were applied to which pixels. In preferred embodiments, such spatial control can be achieved by mechanically applying solution only to certain regions of the chip, instead of applying solution to the entire chip pixel array. In other preferred embodiments, this can be achieved by applying a probe assembly solution to the entire chip array, but using a voltage driven assembly processes such that only electrically activated pixels will assembly probe molecules into the sensor nanoelectrodes. In preferred embodiments, this could be done by applying a voltage to electrodes that either attracts or repels the probes or probe-bridge complexes, such as using a positive voltage to attract the negative chard on DNA in solution, or a negative voltage to repel such DNA. This relies on the well-known process of electrophoresis. In other preferred embodiments, an AC voltage may be used to selectively attract or repel the probe or probe-bridge molecules, using the well-known process of dielectrophoretic forcing. In particular, in one preferred embodiment, the solution contains the particular probe type or probe-bridge type for a particular target is applied to the solution, for a short period of time, and in a low concentration, such that diffusive transport is unlikely to deliver these molecules to bind to nanoelectrodes on the chip array. However, for the desired nanoelectrode locations of the molecules, an AC voltage of proper frequency and amplitude to create a dielectrophoretic force that will drive these molecules to concentrate near the electrode gaps of the intended sites, allowing them to selectively bind the intended probes or probe-bridge complexes. The solution is then flushed away, and the next probe may be introduced, similarly target sites for it. This may be done for individual probes, or pools of distinct probe types, in which case their locations are restricted to a much smaller set of possible sites, but probe type from the pool is still randomly distributed across electrodes within those site sets, and further location information would be required to complete the map to the individual sensor level. In preferred embodiments, the low concentrations of the probes used may be in the range of 1 pM (pico-Molar) to 100 nM (nano-molar), the exposure time used may be in the range of 0.1 s to 100s, and amplitude of voltage used maybe be in the range of 0.1V to 10 V, and the frequency of AC modulation used may be in the range of 1 kHz to 100 MHz.

In other preferred embodiments, the probe map may be constructed by a process of decoding hybridization probe locations using the result of special binding reactions, with special detectable probes (not necessarily DNA hybridization probes) that are designed to be able to locate or localize each specific hybridization probes type on the array. In this approach, each distinct hybridization probe type is provided with one or more binding sites, directly coupled to or integrated into the probe molecule (or, in preferred embodiments, to the probe-bridge molecular complex, pre-formed), and where such sites are capable of producing an observable binding signature in response to binding with the corresponding decoding probe molecules, and with such a signature being localizable to the resolution of a specific probe site (nanoelectrode pair) or pixel, as required for the complete hybridization probe map. One or a series of these decoding signatures can thereby be affiliated to a specific probe site on the array, and these may be used to decode which particular type of hybridization probe is present at the site. In this method, the hybridization probes (or probe-bridge complexes) are applied as a pool to the pixel array, allowing them to randomly assemble into the nanoelectrodes on the pixels, and after they are so assembled, in a series of subsequent reactions, known decoding probe molecules are applied to the array and observing for the production of their unique binding signatures, localized to sensor sites. For each site, there is at the end of this procedure a resulting series signals localized to that sight, that are sufficient in combination to determine confidently which hybridization probe must be present at the site.

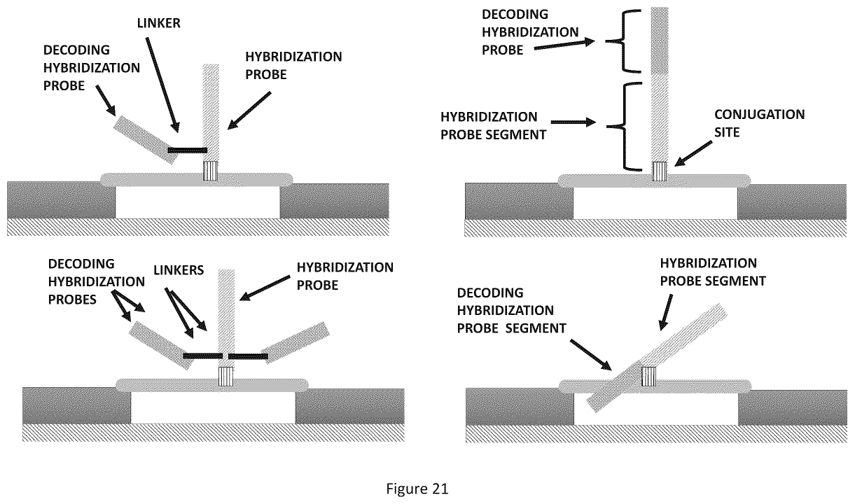

Such observation of a decoding signature in preferred embodiments may be done by monitoring electrical signals from the pixels and respective sensor sites, that are produced by the binding of the decoding probe. In preferred embodiments, these decoding probes are themselves DNA hybridization probes, whose targets are affiliated with the hybridization probes, and their hybridization events also can produce detectable signals in the sensor. The decoding scheme may require one or multiple such decoding hybridization targets affiliated with the hybridization probe. There are many ways such targets can be affiliated with a hybridization probe, FIG. 21 shows four representative examples, and many other schemes can be readily extrapolated from these specific examples. For example, the upper left embodiment in FIG. 21 shows that the DNA target of a decoding probe is linked to the primary hybridization probe DNA by a linker molecule, and the lower left figure illustrates that there could be two or more such linked targets. Generalizing this, in that way, any number of decoding targets can be affiliated with the probe to be encoded. For another example, the embodiment in the upper right of FIG. 21 shows that a segment of the DNA strand that contains the hybridization probe, could also include another contiguous segment that is a target of the decoding probe and, by generalization, and number of such targets could be present in series, all encompassed in a single DNA strand. The figure in the lower right illustrates that the conjugation of such a strand to the bride could be at an interior part of the strand, in order to allow both the hybridization probe segment, and the decoding target segments, to be similarly close to the bridge, for more sensitive detection of the decoding targets.

In other preferred embodiments, these signatures of the decoding probes may be optical signatures from dye molecules or fluorescent groups, such as Quantum Dots, on the decoding probes, and such signatures may be acquired by microscopic imaging of the chip, under white light or fluorescent light, and widefield imaging or confocal scanning conditions. This is sufficient to localize the optical signals to within approximately 1 micron, or one wavelength of the emitted light, of the location of the decoding probe itself, therefor providing spatial resolution in the range of approximately 0.5 microns to 1 micron. This is sufficient to localize the probes in preferred embodiments. If well-known super resolution imaging methods are employed, localization below the wavelength of light (the so-called diffraction limit) is possible, down to 100 nm or down to 20 nm. This is sufficient resolution to localize probes many preferred embodiments using such optically labelled

One preferred embodiment of a decoding method to produce the probe map is “direct decoding”, in which individual decoding probes specific to the individual hybridization probes are used to directly locate the sites of each probe type on the array. In a preferred embodiment, the decoding probes are hybridization probes. Assuming there are N hybridization probes types H1, H2, . . . Hi, . . . HN, in this method there are provided N decoding hybridization probes D1, D2, . . . , Di, . . . , DN. These should have distinct sequence targets, that have very low cross-hybridization between them. A target oligo for Di is physically affiliated to probe Pi, for I=1 . . . N, such as by any of the means represented in FIG. 21. These probes are assembled randomly onto the sites on the chip array. Then, a series of N hybridization reactions is performed on the chip. In reaction i, decoding probe Di is hybridized to the array, and the detection readout of each sensor is recorded Sensors where Di finds it target produce a detection signal, and this directly identifies which sensors have probe Pi. Do this for reactions I=1 . . . N decodes the locations of all probes, and thus provides the probe map. In preferred embodiments, this same method works if instead of electrical readout, optical labeling and imaging location is used to localize the decoding probes. In one preferred embodiment of this, the decoding probes could simply be taken to be the targets of the hybridization probes in question, i.e. Di is the target of HI. The has the advantage that not extra target sequences need to be added to the hybridization probes to achieve this decoding. However, in other preferred embodiments, the decoding probes Di may be chosen to have better physical hybridization properties, than that simple choice affords, such as stronger on-target binding and weaker cross-hybridization with other decoding probe targets, or more uniform such performance across the set.

Another family of preferred embodiments for decoding methods to produce the probe map is generally termed “combinatorial encoding and decoding”. In these embodiments, a series of decoding probe reactions are applied, and for each given probe site on the array, the series of detection/non-detection results from these reactions provides enough information in aggregate to uniquely determine the identity of the probe at the site. Several canonical exemplar embodiments of such combinatorial methods are given here. It is understood that there are many variations, reformulations, and combinations of these provided canonical exemplars that can be used as alternative decoding schemes for building the probe map, and which would also be obvious, from the canonical examples, to one skilled in the theory of codes. All such obvious variations, reformulations, and combinations are meant to be encompassed by these canonical exemplar embodiments.

The canonical combinatorial decoding embodiments provided may be described succinctly and efficiently as follows, wherein to achieve this, the assay to be performed, their order, and their outcomes are arranged and represented with 0/1 in way that allows the direct relation of decoding probe assay results to probe identification codes. Assume there are N hybridization probes types H1, H2, . . . Hi, . . . HN for which a location map is desired. In various preferred embodiments of this method, there is provided a set of N distinct K-bit binary code strings {B1, B2, . . Bi, . . . BN}, where these Bi are various strings of length K, composed of the symbols “0” and “1”, such as such as for example might be the string B=“1001011”, in a case where the length is K=7. The code Bi is assigned to probe Hi, for i=1, . . . , N, and these codes will be used in physical encoding and decoding process to identify this probe for the probe map on the array. Note that any such set of N such strings will provide a valid encoding for the methods that follow, although special sets of such strings, as described in preferred embodiments below, can also provide for the additional feature of error detection and correction in the decoding measurement process used in array assays. Also, note that as there are exactly 2 K distinct strings of length K, so it is required, in order to have enough such binary codes, that that 2 K≥N. Indeed, for any K satisfying this, preferred embodiments include the choice of any subset set of N strings from the master set of 2 K possibilities, and if N=2\hat{ }K, a preferred embodiment is simply to use all K-bit strings, listed in any order. Note that in these code assignments, if all the code strings {B1, B2, . . Bi, . . . BN} have the same binary digit in position j (i.e. the jth digit is always 0, or always 1), this position is uninformative and can be eliminated from the strings, reducing their length K to K-1. This can be repeated to remove all such uninformative positions in the strings, so as to reduce the number of physical encoding probes required in the methods below.

In one family preferred embodiments, there is further provided a set of 2K decoding probes that are hybridization probes denoted as Dij, where i=0 or 1, and j=1 . . . K. These decoding probes should have distinct target sequences, and preferably low potential for cross-hybridization. For the probe Hi, the associated physical encoding targets are taken to be the target DNA oligos of the encoding probes D(b1)1, D(b2)2, . . . , D(bK)K, where here b1, b2, . . . , bK are the binary digits of the encoding string Bi, i.e. bj is the jth digit of string Bi. These encoding probe target DNA oligos are then to be physically linked or affiliated with the physical hybridization probe oligo, such as by the methods illustrated in FIG. 21, or other means. Note that with the probes so encoded, that for any probe, Hi, and a pair of encoding probes D0j and D1j, precisely one of these two probes will have its target on the physically encoded probe for Hi. To achieve decoding of probe locations on the arrays, the series of 2K reactions trying to hybridize the individual encoding probes D01, D11, D02, D12, . . . D0j, Dij, . . . , D0K, D1K is performed, and for any probe site of the array, the outcome of these reactions is recorded by taking the trial of both D0j and Dij, and recording the outcome of these two reactions for the site as trialj=0 if D0j bound or trialj=1 if Dij bound, at the site in question. Then the complete binary string of trial outcomes for the site in question is succinctly written as (trial1)(trial2) . . . (trialk). As constructed in this process, this string will be identical to the code Bi assigned to the hybridization probe Hi that is in fact located at the site in question. In this way, for each site, the reaction results from the decoding probes are decoded to some precise Bi code and related Hi probe from the probe set. Therefore, the outcomes of this series of 2K decoding hybridizations across all sites on the array provides the code strings that identify and localizes in the array all occurrences of every probe H1 . . . HN. Thus, the probe map is constructed.

In another family of preferred embodiments, the situation is as in the above embodiments, but the physical encoding is done in a more compact form: For the probe Hi, the associated physical encoding targets are taken to be the target DNA oligos of the encoding probes D(b1)1, D(b2)2, . . . , D(bK)K, but only tag the probe physically with the D1×targets, ie. Do not tag them with any of the DOx targets, and when doing the decoding above, apply only the K reactions of the probe D1×probes, D11, D12, . . . Dij, . . . , D1K. The results of these trial assays can be recorded as testj=1 if Dij binds at a probe site, and testj=0 if it does not bind. In this case, the result string (test1)(test2) . . . (testj) . . . (testK) is the same binary string as recovered above in the previous embodiment, because above, if D1j did bind, testj=1, as in the present method, and if D1j did not bind, this is the same as D0j binding, which also recorded as 0 above and in the present method. Thus, the same probe map decodind is achieved. It is a benefit of this embodiment that fewer physical target oligos need to be linked to each hybridization probes, and overall, the method requires only half as many physical encoding probes to be produced, and their associated targets to be produced and linked to probes.

Another family of preferred embodiments of methods for making probe location maps may be described efficiently and succinctly as follows. Again, reaction procedures and outcomes are efficiently encoded by 0/1 indicators that allows direct interpretation of decoding assay results for an unknown probe as the binary code identifying the probe. for This method relies on reacting pools of decoding probes, rather than individual probe reactions, within otherwise a similar logical framework. Assume there are hybridization probe types H1, . . . , HN, with assigned K-bit binary codes {B1, . . . , BN}. There are then further provided the same number of N decoding probes that are hybridization probes denoted as D1, . . . , DN. These decoding probes should have distinct target sequences, and preferably low potential for cross-hybridization. The target of each Di is to be physically linked to the corresponding probe Hi, such as by the means illustrated in FIG. 21. A total of 2K Pools of decoding probes P01, P11, P02, P12, . . . P0i, P1i, . . . , P0K, P1K, are defined as follows: The members of pool P0j are all probes Di for which bit j of code Bi is 1, and similarly, the members of pool P1j are all probes Di for which bit j of code Bi is 0. Given these pool memberships, the corresponding physical probe pooled are produce, as equimolar mixtures of the decoding probe oligoes for the pool. Under this construction, note the result of reacting the physical pool of probes P1j against a probe Hi on the array will be a match if code Bi has 1 in position j, and this outcome is to be recorded as trailj=1, while otherwise, if there is a 0 in position j off code Bi, the match will instead occur for pool P0j, and this outcome is denoted by trialj=0. Let the outcome of all the K pooled reactions against the array be recorded by the string (trial1)(trial2) . . . (trialK). Then this string matches the code Bi of the probe in question Hi, and these series of reaction outcomes there provides the decoding of the probe identity. The results of reacting the 2K pools to the array, therefore, decodes all occurrences of all probes on the array, and provides the required probe map. Note in one preferred embodiment above, the Di could be taken as the targets of the Hi, in which case no special linkage of targets to the Hi is required. However, in general, other sets of {Di} could have more desirable hybridization properties of uniformity of Tm and low cross-hybridization potential, and better discrimination of perfect match signals from background.

In another family of preferred embodiments, the situation is as in the above embodiments, but the physical encoding is done in a more compact form as follows. Only the K pools P11, P12, . . . , P1j, . . . P1K are physically constructed, and these are reacted to the array, in a series of K reactions, and for each site on the array, the result is recorded as trialj=1 if hybridization was observed with pool P1j, other 0 if it was not observed. The resulting string (trial1)(trial2) . . . (trialK) that encodes this outcome, is identical to the string in the above embodiments, and therefore this string provides the code string Bi that identifies the probe Hi. The results of reacting these K pools to the array, therefore, decodes all occurrences of all probes on the array, and provides the required probe map. This requires half as many pool constructions and hybridizations as the previous embodiments.

Preferred embodiments for Error Detection and Correction in Probe Mapping. As noted, in the above embodiments of decoding methods, any set of N binary K-bit strings (B1, . . . , BN) provides an encoding and decoding method, as so a great number of possible methodologies are outline above. Within this framework, the specification of specific code word sets for preferred embodiments can provide substantial benefits. For illustration of this point, note that in the combinatorial decoding schemes above, if the number of probes is N=2\hat{ }K, each and every K-bit binary string is then necessarily assigned a probe, in 1-to-1 fashion. However, in this minimal code length K scenario, if an error were made in measuring the code of a probe in the above methods, it would produce the code of a different probe, since all codes are used, and thus the result in incorrect decoding. Allowing a larger binary coding string length K than the minimum required allows for robustness against such errors. Specifically, it is possible that the set of binary codes {B1, B2, . . . , BK} is chosen as a set that allows for error correction or detection, such that if a code string from this set were corrupted by one or more bit flipping errors, it is possible to determine such corruption has occurred, and with some encodings, also to correct it back to the uncorrupted state, error free. This will provide for protection against errors that could be made in the decoding measurement process outlined above, in the form of an false detection of hybridization (error of 0→1), or missing a true hybridization (error of 1→0), so that such errors do not lead to incorrect or indeterminate decoding of probe identity. Many such error correction or error detection encodings are known to those skilled in the art of error correction methods for binary data. In some preference embodiments, one such method is the use of binary strings that add one or more parity bits add the end of an initial given string, which provide power to detect or correct certain errors. Another preferred embodiment is the use of Hamming Codes and Hamming distance to detect and correct errors. In this class of methods, the assigned number N of code words must be only a small fraction of all possible binary codes of length K, and the precise code words are taken to have highly distinct bit sequences, such as, for example, this could be N randomly selected code words from all 2\hat{ }K»N. In such a case, if there is a corrupted code, it may be detected because it does not match any of the assigned codes, and it can be corrected back to the closest of the allowed assigned code strings, with closeness measured by the Hamming Distance (number of mismatches between the digits of two binary strings). This general technique always affords some power for error correction of at least limited number of bit errors, and for any proposed set of code words, {B1, . . . , BN}, the error correction properties of this can be directly and exactly assessed by brute for examination of all possible corrupted versions of each Bi, and noting for which corruptions this process corrects them. Preferred embodiments of such methods are provided by specific Hamming Codes, which are strings sets {B1, . . . BN} that have optimal or highly effective and uniform error correction by this means of correcting to the Hamming distance closest allowed code. In general, many other error correction encoding schemes are known to those skilled in the art of coding theory, and any of these schemes defined for K-bit strings can be used to provide K-mer code word sets that also have powerful error correction capabilities, and which can be used here to correct for possible decoding hybridization errors. In general, this provides a mechanism with arbitrarily good power to correct errors, at the cost of larger K—and therefore more physical decoding probes and more decoding reactions).

In preferred embodiments, the decoding probes used in the above decoding methods, electrical or optical, are shorter oligos, such as in the range of 8-25 bases, and any two such targets have multiple mismatches between them, to reduce cross-hybridization, preferable 2 or more, and preferably 4 or more. In preferred embodiments, they may be PNA probes, so that a short probe can have stronger binding and higher Tm, and the impact of single mismatches can be greater on reducing cross-hybridization. In preferred embodiments, all of the methods disclosed above can be used with electronic detection of decoding probe hybridization provided by the sensor chip array, or, in other preferred embodiments, using optically labeled decoding probes-such as a dye label or Quantum dot label, or gold nanoparticle label, or any other label detectable by microscopy and compatible with attachment to a single molecule DNA oligo—and localization of probe binding by microscopic imaging.

In another preferred embodiment of the above decoding methodologies, the objective and benefit is to have a decoding method in which the number of decoding targets added to each probe is a number J that can be specified as desired, so as to control the amount or of hybridization target added to the probes for decoding purposes. This can be achieved as follows, using the compact form of the first family of preferred methods above: the binary codes string {B1, . . . , BN} are defined as follows: for the set of numbers (1, . . . , L), for some L, represent a subset S of this set by the K-bit string (b1)(b2) . . . (bK), where bi=1 in i is in the subset S, and 0 if not. This is the sometimes called the indicator function for the subset. E.g., the subset (2,4) would have indicator string 0101000 . . . 0. There are 2 K such strings, corresponding to the membership indicator strings of all 2 K subsets of S. In the setting, define as the codes the set of all strings that have exactly J 1's in them. The number of such strings is known in combinatorics as “L choose J”, and is N=L!/(J! (L−J)!), where “n!” denotes n factorial=n×n−1× . . . ×2×1. When this set of code strings is used in specified “compact” forms of the methods above, this has the advantage that for the physical encoding, wherein a target is added for every 1 occurring in the encoding string Bi, there are always exactly J such 1's, and so exactly J hybridization targets are added to encode each hybridization probe. This therefore has the advantage of controlling the amount of target material added for decoding, to be J oligo targets. For any desired number of hybridization probes N to be encoded, and any desired J>1, L can be chosen large enough to that L!/(J! (L−J)!) in >=N, and therefore provides enough such codes. The cost of achieving this as that L encoding probes are required. For example, suppose there were N=1024 hybridization probe types. One option would be to take all K=10-bit binary strings, and assigns all these as codes. However, in the above methods, each probe would get linked to either 10 targets (in the non-compact scheme), or a variable number of targets between 0 and 10 in the compact schemes. The decoding would require 20 reactions in the full scheme, or 10 in the compact scheme. However, restricted to linking to J=2 targets per probe, L=46 encoding probes and reactions are required, but allowing J=3 reduces this to L=20, and J=4 allows L=15. These are generally more desirable, such as required 15 probes and reactions, but only needing to add 4 decoding oligo targets to each probe. However, these do not provide any error correction capability, as a single bit error would produce a 3 element or 5 element subset indicator string, which does not have a unique Hamming distance closest string in 4 element set indicators.

Chip-Based Systems In preferred embodiments, the disclosed chip-based multiplex hybridization probe sensor devices are deployed in a compact, low cost electronic instrument that is suitable for distributed use, field use, or point-of-care use. Such instrument architectures in preferred embodiments comprise a chip board that mates to the chip, motherboard that hosts the chip, and FPGA-based control and data transfer subsystem, a data processing subsystem, which may comprise CPUs, GPUs, FPGAs or other signal processing hardware, a fluidics subsystem, on instrument data storage, and of-instrument data transfer systems.

In preferred embodiments, this chip device is deployed into a cartridge that in preferred embodiments also allows for some or all other liquid reagents or input sample required for operation to be on-cartridge, to allow for a partial or fully dry instrument platform. In preferred embodiments, this cartridge is run on a desktop instrument that provides for a user interface, a control computer controlling chip and system functions, control of any on-board fluidics or actuators that control on-cartridge fluidics to supply sample and reagents to the chip, transfer of data from the chip to internal storage or data processors, such as FPGA, GPU or CPU data processors, and transfer of data off instrument via direct internet or wireless connectivity to remote or cloud-based data centers, and such system also provides a sample prep system, internally or as a companion instrument, that takes biosamples of interest and coverts them to the form for on-chip application. In preferred embodiments, such a system can have a compact form factor suitable for mobile use. In other preferred embodiments, such a system can have a highly compact form factor suitable for point-of-use or point-of-care or in the field deployment. Such point-of-use applications in preferred embodiments would include testing stations deployed at airports, transportation hubs, hospitals, schools, stadiums, cruise ships, transport chips, or other major sites of congregation, or deployed at site of business or commercial activity. In preferred embodiments, such testing stations would be deployed for use in the home, for personal testing and monitoring. In preferred embodiments, point-of-use systems may be deployed in the field for military, police, customs or border control point-of-contact testing, or other in-the field testing and monitoring applications, such as testing of commercial vehicles, trains or aircraft for presence of pathogens.

Tag Reporter Assays For many types of DNA targets detection applications, it is possible to have the detection response encoded by the production of short segment of synthetic single stranded DNA oligo, or a “DNA tag”, such that the detection of the target is represented by the presence of the tag, and the relative abundance of the target may further be represented by the relative abundance of the tag. Thus, the DNA tag becomes a reporter, that can be used to readout the assay results. This can be generalized beyond the detection of DNA targets: a broad array of other molecular detection applications can be formulated in assays that represent the detection results via a reporter DNA tag. There are multiple benefits to such tag reporter assays, including: (1) the assay results can be read out by hybridization sensors that have the tag as a target, thereby allowing a universal, simple and convenient readout sensor for many diverse molecular detection assays; (2) tags can be designed for optimal and predictable hybridization performance with the sensor, unlike trying to make hybridization probes constrained by native DNA target sequences of interest in the primary detection assay, which are more variable in properties when used as hybridization probes; (3) tag reporters fundamentally allow for a very high degree of multiplexing, since the number of distinguishable DNA tag types is essentially unlimited, and hybridization sensor arrays provide for multiplex readout, unlike other common forms of reporters, such as dye labels, which are severely limited in distinguishable colors or dye spectra or the ability to group multiple dye molecules into a label, and which require increasing complex optical systems for multiplex readout; (4) tag reporters allow the primary detection reaction to take place under standard and optimal solution phase conditions, instead of performing primary detection under the less ideal conditions that may exist near the molecular electronics sensor (proximity of surfaces, other sensor molecular components, and varying voltages and charges transfer, etc.); (5) DNA tags can be easily amplified by PCR reactions, so there is a simple and convenient way to amplify the reporter signal as needed for more sensitive detection, and this provides sensitivity down to the single target molecule detection limit.

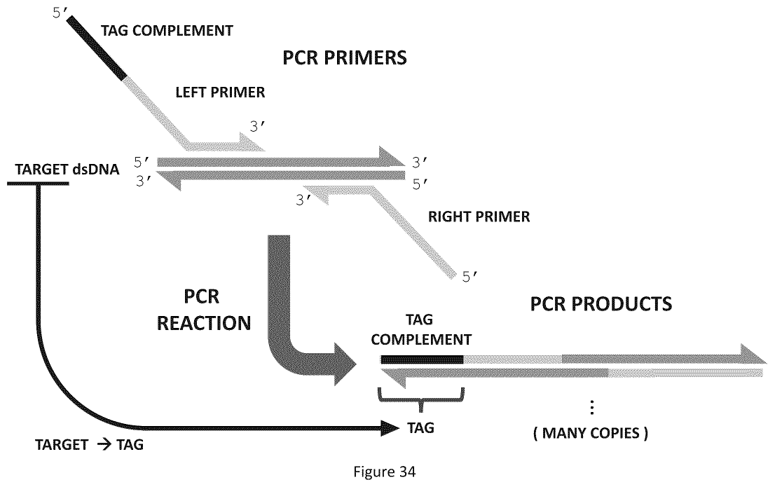

Examples of preferred embodiments of tag reporter assays are illustrated in FIGS. 34 through 39. FIG. 34 shows an example of detection of a target DNA segment, from within a complex sample pool having unknown and possible diverse segments. The target segment shown there as a double stranded DNA (dsDNA) segment (but this could alternatively be a single strand DNA (ssDNA) target as well). The detection is achieved by having PCR primer pair targeting the segment, indicated as “left” and “right” primers, and these primers in general have 5′ “tails”, which will be incorporated into the PCR products. In order to produce a tag reporter, for any give tag sequence, one of the tails (here the “left” primer) contains the complement of the given tag (i.e. reverse complement), as indicated by the black segment. If the PCR primers find their target, and the polymerase extension reactions proceed, the result amplified PCR products, shown, contain the primers, and thus the tag complement, and the synthesized complementary strand, as show therefore contains the tag of interest. In this assay embodiment, note the oligo single strand tag of interest does not exist in the reagents put into the assay. The single stranded tag only exists if it is produced by the PCR reaction finding its target. Further, the PCR reaction will produce products in proportion to the amount of target for some range of product production, until the reaction becomes limited by limiting amounts of primers, polymerase, or nucleotides, or by interference from the amount of product DNA produced. Thus, the reaction can be performed with conditions such that the amount of tag produced is directly and quantitatively related to the amount of target present, providing for quantitation of the target concentration. Alternatively, the PCR reaction can be driven to equilibrium or endpoint, producing a limiting amount of product, to provide a simple “yes/no” detection of the target with maximal sensitivity. The net effect, as indicated by the arrow from target to tag, is that detection of the target as reported in tag production. Clearly in this example, the tag could be any sequence segment compatible with being on the tail of a PCR primer. This places very little constraint on the DNA tag, and lengths of 10-100 bases would readily be possible, with very little restriction on the sequence compositions. This illustrates that the number of such distinct tags sequence options is formally 4¹⁰-4¹⁰⁰, and therefore practically unlimited, as millions (10-mer), billions (15-mer), trillions (20-mer) or more distinct tag options are readily available. This also implies that a great deal of selection can be applied to select tag sequences that have desirable properties for assay performance or detection, such as optimization of hybridization properties, and keeping the length relatively short in this 10-100 range. The PCR reaction indicated in FIG. 34 could be any of the large number of known PCR-type reactions, that reply on a primers, and can tail in sequences, such as the classical thermal cycling and its variations, as well as variations that are isothermal, such as PCR with denaturing chemical cycling, loop-mediated isothermal amplification (LAMP), helicase-dependent amplification (HDA), and recombinase polymerase amplification (RPA). Many compatible PCR reactions and many variations on such reactions are known to those skilled in nucleic acid manipulation.

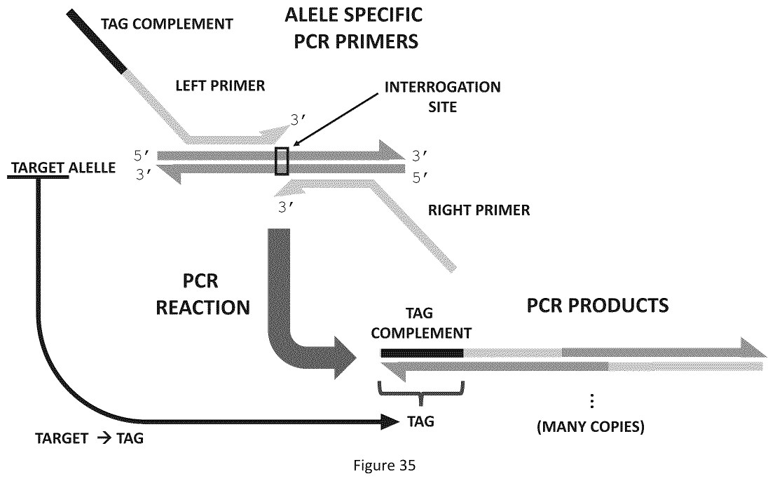

FIG. 34 shows another preferred embodiment, which is as described for FIG. 34, except that the detection target is a DNA segment that differs only by a single letter at the interrogation site indicated, from other DNA segments that may be present in the sample pool of DNA fragments. In this case, as indicated, the PCR primers used are allele specific. In this case, the tag reporter assay can therefore report on the presence of a specific Single Nucleotide Polymorphism (SNP) variant allele, or other variant alleles which may differ by very minor sequence changes, such as small insertions or deletions in the range of one to a few bases. Detecting and discriminating such minor variations is essential to genotyping or strain identification applications. As indicated by FIG. 35, the allele-specific PCR primers are designed such that the 3′ end of at least one, or in the preferred embodiment shown, both, primers will only properly hybridize to the target if they match the allele of interest. Because the polymerase extension in PCR is very unlikely if the 3′ end of the primer is not matched to the template, such allele-specific PCR is highly specific to the target allele of interest, even if it differs by just one base from other fragments present.

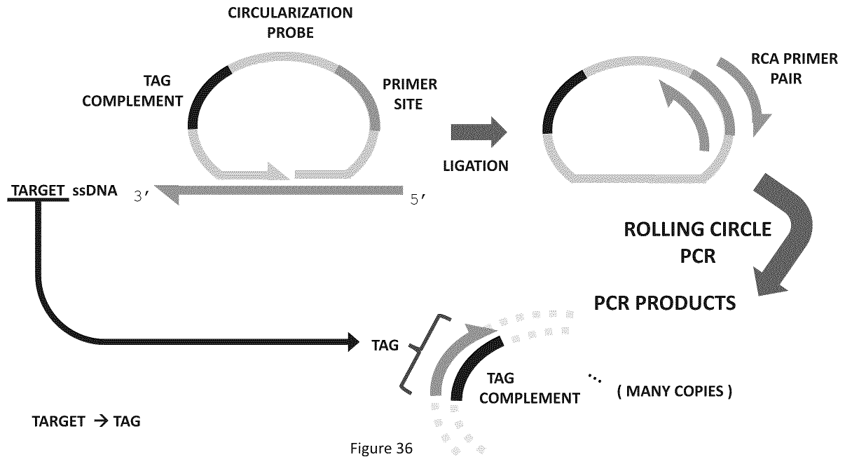

FIG. 36 shows another preferred embodiment, where the detection target of interest is a segment of single stranded DNA (ssDNA). Here, the “circularization probe” shown is a linear single stranded DNA, for which both end match (by hybridization) to segments of the target strand. This linear probe with therefore hybridize to the target strand, bringing its 3′ and 5′ end together, and abutting, such that the probe can be ligated into a circular DNA by the reaction of a suitable ligase enzyme, many of which are well known and widely used for such DNA manipulations (e.g. T4 ligase, T7 ligase, etc.). As shown, the circularization probe is designed to have the complement of the desired tag as a segment, shown in black, and it also has a primer site, for a Rolling Circle Amplification (RCA) primer pair. RCA is a well-known PCR technique, where, with such a circular templates, and primer pair, and a strand-displacing polymerase, it will “roll out” a long linear complementary copy as it extends the primer and goes around the circle many times, and the opposing primer will prime off of that copy at multiple sites, and this process iterates, resulting in a real-time, isothermal production of a branched structure containing many long, linear, double-stranded copies of the circular template. As a result, as shown the PCR products indicated contain many copies of the tag itself, produced on the strand complementing the template strand. Note that the desired single stranded tag sequence does not exist anywhere in the initial assay reagents-only its complement is present there—and so the reporter tag is present only if the original target was detected by the circularization process. In preferred embodiments of this process, the RCA PCR proceeds to equilibrium quickly, in minutes, and this provides a rapid “yes/no” detection of the target with single molecule sensitivity. In other preferred embodiments, this reaction can be arrested before this endpoint, such that the amount of tag product can be used to assess the amount of target.

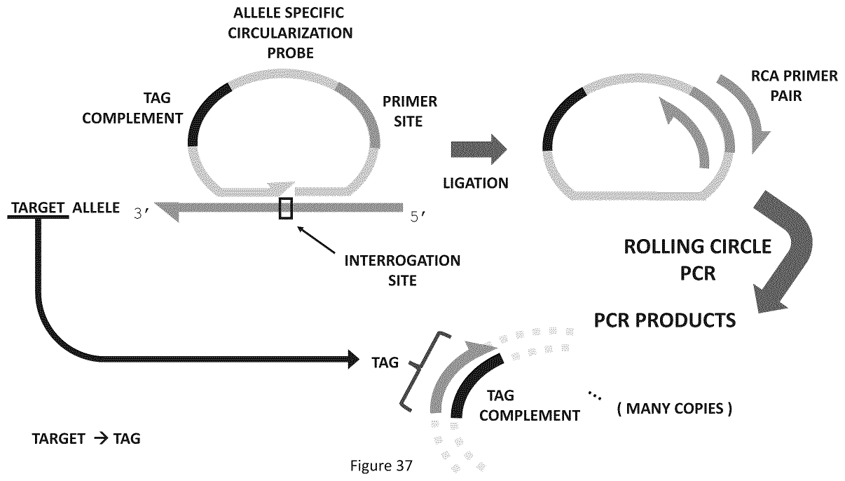

This circularizing ligation can be highly specific to the probe end segments exactly matching the target strand of interest. As shown in FIG. 37, the circularization probe can in particular be allele specific for a single base difference at the 3′ or 5′ end of the probe, or other minor allelic variation, since the ligation is very selective for a perfect match against the template for the ends to be ligated, the base adjacent to the point of ligation. In another preferred embodiment of this method, the ends of the probes do not meet, but instead have a gap of some number of bases between them, which must first be filled by polymerase extension of the 3′ end, to meet the 5′ end, before ligation can occur by the action of a ligase. This gap filling version can have increased specificity, since the polymerase and ligase both perform checks on proper probe hybridization matching to the target template. Many other variations on the circularization process and Rolling Circle Amplification PCR are known to those skilled in nucleic acid manipulation, and such obvious variations could also be used in this tag reporter assay. Another variation is to perform the circularization as primary detection, as indicated, then degrade away all remaining linear DNA using exonuclease that attacks the ends of strands (such as mung bean exonuclease), at which point only circles remain, and these can either be amplified by RCA, or can be cut (using restriction enzymes, chemical cleavage, or other means to induce breaks) to linear segments, and amplified by other PCR methods. This can, for example, provide benefits of purification of materials, for less production of unwanted or off-target polymerase extension products, as well as a greater linear response range for other forms of PCR, versus RCA, in order to quantify the amount of target.

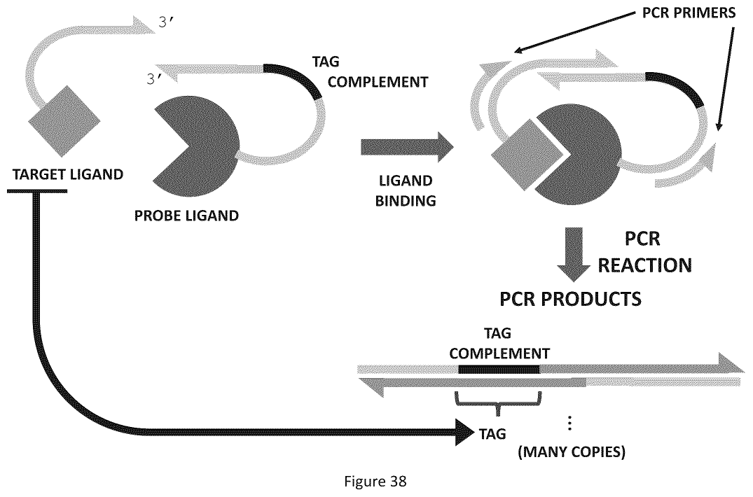

FIG. 38 illustrates a detection application where the target is not a DNA segment, but in general could be any type of analyte, such as a protein, an antibody, an antigen, a biomolecule, a small molecule, a chemical compound, a nanoparticle, or a larger complex such as a cell or cellular component, as well as nucleic acids. In the scenario shown, there is a given probe ligand, and the goal of the detection is to determine if it the probe ligand binds or interacts with a candidate target ligand. In this assay, the probe ligand is prepared so that attached to it is a single stranded DNA with a 3′ end exposed, as indicated. The attachment may generally be done by any of many standard and well-known means of bioconjugation, appropriate for the type of analyte in question. The DNA strand on the probe also has a tag complement on it, as shown in black, which will report the tag if the probe binds the candidate target. The candidate target ligand is also prepared with a single stranded DNA attached, with exposed 3′ end, which is complementary to a segment at the 3′ end of the test ligand, as depicted by the shading. If the probe ligand binds or has a sufficiently strong interaction with the candidate ligand so prepared, the two attached strands will hybridize as indicated along the common matched 3′ end segment. It is presumed this hybridization is otherwise much less frequent or stable, without the ligand interaction, which can be achieved by adjusting the melting point of this hybridization to be low enough. A polymerase extension reaction is then performed, in which case the 3′ ends will both extend and create double stranded DNA, and in so doing, the tag strand will be synthesized from its complement on the probe ligand strand. Further, the DNA strands on the ligands are to have PCR primer sites located and oriented as indicated, and such that the primer extension will also create the proper templates for the primer binding, which were not present before extension. After this point, if such primers are added or present, a PCR reaction will be able to amplify the extended fragments from both the probe ligand and candidate target ligand, resulting in the PCR amplification products indicated. These contain the tag complement, and the tag itself on the complementary strand, thereby produced the tag reporter, in amplified quantity. If the PCR reaction is run to equilibrium or endpoint, this provides a “yes/no” detection that the probe ligand found a binding target. If this PCR is stopped short of equilibrium, the amount of tag produced provides a quantitative measure of the amount of the candidate ligand present. In another preferred embodiment of this method, instead of using polymerase extension to effect joining of primer sites on probe and target ligands onto one strand to enable PCR amplification, this joining could instead be a ligation reaction between the DNA strands on test and candidate ligand (which may be double or single stranded in accord with appropriate ligation methods) which would still result in a PCR-amplifiable ligation product. Preferred methods for performing this ligation are illustrated by the strand joining methods illustrated in FIG. 40 (ignoring the A:B tag illustration there) and which are further described below, to form a complete strand that has both PCR primer sites represented on it. In general, to those skilled in the art of nucleic acid manipulation, from the example given, many variations in methods would be obvious that could be employed to result in an amplified product if probe and target ligands bring their respective strands into proximity for extended time above the background levels of proximity in the solution, and if processes of polymerase extension and/or ligation are used to join these two partial strands into one amplifiable strand comprising the two requisite PCR primer sites. Other obvious variants could involve the production of a circular template from the joining of two parts, for the purpose of using Rolling Circle PCR.

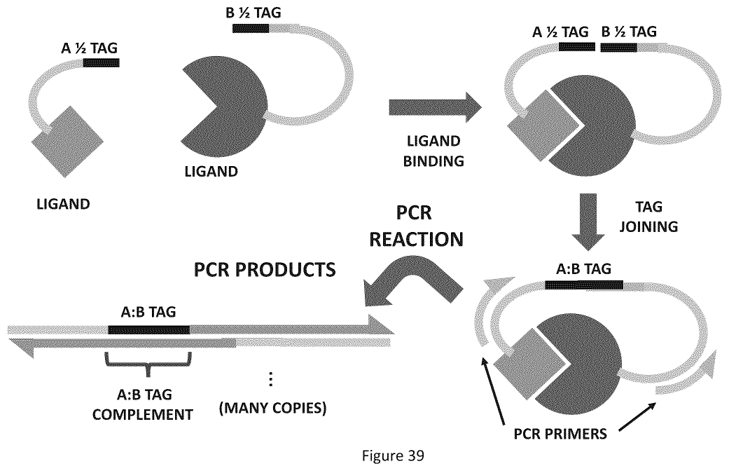

FIG. 39 illustrates a detection application where again the targets of the detection are not DNA, but in general could be any type of analyte, such as a protein, an antibody, an antigen, a biomolecule, a small molecule, a chemical compound, a nanoparticle, or a larger complex such as a cell or cellular component, as well as nucleic acids. The purpose of the assay shown is to determine if two possible ligands binds or interacts with each other. This is sometimes called a ligand-ligand interaction assay, or a protein-protein interaction assay in the special case of proteins that my interact. As indicated, the two candidate ligands are prepared to have strands DNA attached. These attachments may generally be done by any of many standard and well-known means of bioconjugation, appropriate for the type of analytes in question. Further, one ligand has an “½ tag” segment at the end of its DNA, of type “A”, and the other has a “½ tag” segment of type “B”, where these type A and B ends are such that they can be joined to each other by some suitable joining reaction. As shown, if the ligands bind or interact strongly, the two strands are brought into proximity. Through the suitable ligation or other strand-joining such as those preferred options shown illustrated in FIG. 40, the A and B ½ tag segments are joined, to produce the full “A:B” tag that is the reporter for this interaction event. Assuming there are also PCR primer sites as indicated, the resulting ligation product can be PCR amplified, to produce PCR product that contain amplified amounts of both the “A: B” tag and its complement. By creating such a “bipartite” tag, it can encode the identity of each ligand partner, and so this type of tag reporter can be used with multiple candidate A and B ligands allowed to interact in one pool, and report out all possible A ligand vs B ligand interactions. In an alternative preferred embodiment, if ½ tags of A type can join to each other, then just the A type can be used, and the full tags so formed can report out any of the possible ligand-ligand interactions amount a set of ligands. Again, note, as in the other above examples of tag reporters, the complete bipartite reporter tag did not exist in the input assay reagents, and only by the joining process (and perhaps subsequent amplification) was it produced. Thus, this provides a tag report out for the detection of the interactions. In preferred embodiments, his could be a “yes/no” answer, or in other preferred embodiments, it could be used to quantify the overall frequency of such interactions in the ligand pool. FIG. 40 illustrates some of the specific ligation or joining reactions that could be used to form the bipartite tags in the method of FIG. 39. Some of these joining methods can join any end against any end, i.e. they can be used with just a single A type ½ tag, while others can only join ends of different types, A:B. As shown in FIG. 40, these joining methods include: ligation of the blunt ends of single stranded DNA (this has an end type restriction, since a 3′ end must ligate to a 5′ end); blunt end ligation of double stranded DNA (this has no end compatibility restriction, i.e. any such end can ligate to any such end); ligation of double stranded DNA with so-called “sticky end” overhangs, such as the widely used TA overhang shown (or designated [T/A], for a 1-base overhang), or multi-base overhangs, such as the 4-base overhang shown [TAAA/ATTTT] (which can be more efficiently). Note these overhang ligations insert the overhang sequence into the middle of the resulting A:B tag. These specific sequence overhang ligations have end compatibility restrictions that they only ligate to a distinct complementary end, unless the overhang sequences are palindromic (identical to their reverse complement, such as 5′-CG-3′, 5′-AT-3′, 5′-ACGT-3′, 5′-AATT-3′, etc., all such segments necessarily having an even length), in which case such an end can ligate to an identical such end. To otherwise avoid end compatibility restrictions, the overhangs could also consist of universal bases (denoted by “X” in FIG. 40), which can pair with any base or themselves. Such universal bases include Inosine, Nebularine, Nitroindole, and multiple other well-known such bases. The ligations illustrated in FIG. 40 can be performed with many well-known and commercially available Ligases enzymes, such as T4 ligase and T7 ligase, and others. Alternatively, instead of using a ligase enzyme to achieve the join, a polymerase enzyme can be used to effectively join the two disparate DNA segments as well: as shown in the bottom of FIG. 40, if the two DNA single stranded DNA segments overlap and complement at the 3′ ends (here indicated by an illustrative 10 base “N” overlap that is meant to indicate perfect hybridization match of any particular 10 base sequence), then a polymerase primer extension reaction will extend each 3′ end, and effectively produce the joined dsDNA (this also has the end compatibility restriction, defined by the specific 10 base overhang, and this also inserts this base overlap segment into the resulting A:B tag; alternatively, universal base overhangs may be used, with no compatibility restriction). Note that in joining methods that result in a common insert between the unique ½ tags, this will promote cross hybridization amongst the bipartite tags and their respective complementary hybridization probes. This can be controlled by keeping the common insert segment short, preferably 10 bases or less, but such a segment can also be “blocked” during the tag hybridization reaction, by adding in complementary hybridizing oligo for the common segment, to prevent it from otherwise promoting cross hybridization—this is especially useful for longer common inserts, of length 5 bases or more, or which could otherwise have a high Tm for their hybridization. Note in the special case of the use of universal bases, X, as shown in FIG. 40, (e.g. Inosinse, Nebularine, Nitroindole), the probe tags used to capture these by hybridization should use universal bases as well, to complement the universal segment, and further note that during amplification, the universal bases will generally get copied as randomly selected native bases, so the amplified A:B tag will tend to have “N” bases at those sites (any base), and therefore needs to have a universal complement at those variable sites for efficient hybridization.

Considering the processes illustrated in FIGS. 39 and 40, which comprise joining and subsequent amplification of DNA segments, these same joining goals may be achieved via many related variations or combinations of methods of ligation and/or primer extension that are obvious to those skilled in nucleic acid manipulation. Specifically, these obvious variations can be used to achieve the same goal of joining the two ½ tag segments to produce an A:B tag (perhaps with common inserts, as illustrated in FIG. 40). These obvious variations may also be used to achieve the join illustrated in FIG. 38, which as shown there can be understood more generally to be joining via the bottom-most of the diverse joining methods shown in FIG. 40. The other methods illustrated in FIG. 40 provide additional preferred embodiments, as do all such obvious variations on these methods.

The tag reporter assays illustrated in FIGS. 34-40, and described above, are illustrative, and not meant to limit the scope of such assays that produce a reporter tag. From these illustrations, there are many variations and extensions that are obvious to those skilled in the art of nucleic acid manipulation, including many known methods for PCR, amplification, ligation, polymerase primer extension, circularization, and combinations of such methods and refinements of such methods. The examples here illustrate import general principles in such variations, where the single stranded tag reporter itself is not present initially in the assay reagents, and instead the complement is present initially, or parts thereof, and/or parts of the tag may be present, and the full tag is created in the assay by a combination of polymerase building complementary strands, and/or ligase joining together parts, and many combinations of these approaches are obvious to those skilled in the art, and these result in variations on ways to have the assay produce the tag, indicting the primary detection event has occurred. Also, and subsequently, some form of DNA amplification is preferably present, to amplify or produce many copies of the tag after the primary detection process creates the tag. All such obvious variations and alternative combinations of methods are encompassed by these examples.

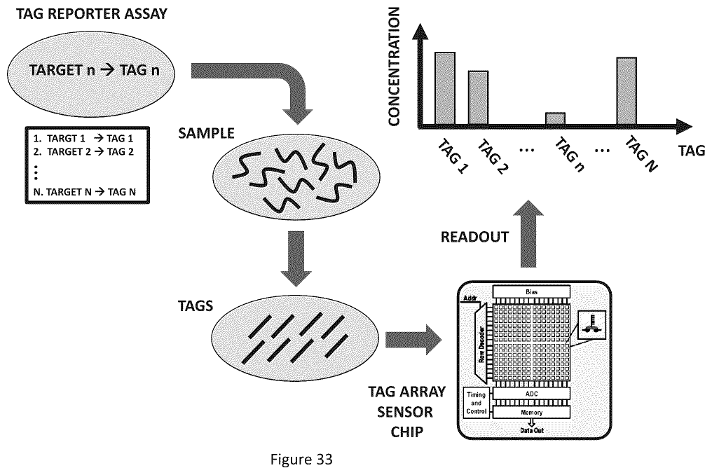

Multiplex Tag Reporter Assays, DNA Tag Sets, and Tag Array Chips The DNA tag reporter assays illustrated above generally can be extended to multiplex versions of each assay, simply by using different reporter tags to report out the different analyte detections. This easily scalable multiplexing is a major advantage of using DNA tag reporters. In preferred embodiments, the resulting different tags generated by the assay are detected by corresponding hybridization sensors in a pooled hybridization reaction. Therefore, in such preferred embodiments, there is a corresponding sensor array chip whose hybridization probes comprise the complements of all the tags that could be generated in the multiplex assay. This preferred embodiment of a multiplex tag reporter assay is illustrated in FIG. 33. As shown there, there is a fundamental tag reporter assay, that represents a target detection by production of a tag. Examples of such a fundamental assay are those illustrated in FIGS. 34 through 39. In the multiplex versions, there are N such targets, and N distinct tags, denoted TAG 1, TAG 2, . . . TAG n, . . . TAG N. The corresponding pool of assay reagents is applied to a complex sample which contains a pool of targets, which may include none, some, or all of the N target set, and in any possible relative concentrations. The result of applying the multiplex assay reagents to the sample is the generation of the reporter tags, which reflect the target detection events and possibly relative abundances of detected targets. The resulting tag set is then applied to the tag sensor array chip, which in preferred embodiments contains multiple instances of the complementary probe for each of the possible N tags, and is produced by the means disclosed previously herein. When the hybridization signals from this sensor chip are collected and analyzed, the result is a net measure of hybridization from each tag (which may be represented by multiple sensors on chip), produced by means disclosed previously herein. According to the relationship between tag hybridization measurements and primary targets for the specific tag reporter assay in use, this method provides for the multiplex assay of the N targets.

In performing this general multiplex method, in preferred embodiments the tags generated from the multiplex assay may be purified before hybridization, to remove unwanted components of the primary reactions, or to exchange into a different buffer for the hybridization reaction. In addition, in preferred embodiments, if the tags contain any common sequence segments that would promote cross-hybridization (such as illustrated in the A:B tags of FIG. 40), complementary “blocking” hybridization oligos may be pre-annealed to the tag pool, or to the array probes, or to both, to preferably occupy such binding sites and thereby block unwanted cross hybridization.

The tag sensor array is a hybridization sensor array of the general type disclosed above, produced by the means described above. The sensor for a given single stranded tag DNA comprises the single stranded complementary sequence for the tag, and is a hybridization sensor as disclosed above. The hybridization probes represented on the array comprise the complements all N tags under consideration. In preferred embodiments, the expected number of sensors for each tag would be in the range of 1-10, or in the range of 10-100, or in the range of 100-1000, or in the range of 1000-10,000. In preferred embodiments, such higher numbers may confer more sensitivity or greater dynamic range of measurement. In other preferred embodiments, the sensor array may be divided using fluidics chambers into sub arrays which represent subsets of the N tag set. In preferred embodiments, there may be 2, up to 4, up to 8, up to 16, up to 32, or up to 64, or more individually fluidically addressable subarrays, each of which contains probes for one or more tags from the N tag set.

In preferred embodiments, the N tag probes are assembled randomly into the array, and which of the tag probes is present at each pixel is decoded by the means disclosed above. In preferred embodiments, the tags themselves are used to do this decoding. In one preferred embodiment, they are serially and individually hybridized to the array, over the course of N serial hybridizations, to directly identify which pixel probes for which tag. In other preferred embodiments, the tags are used to perform combinatoric decoding and mapping of the array, by hybridizing a series of tag pools to the array. In preferred embodiments, each pool contains approximately half of the N tags, and such pools are constructed so that approximately M=Log₂[N] such pooled reactions are sufficient to simultaneously decode the sensor probe identities, as disclosed in more detail previously. This pooled decoding approach is preferable to the serial decoding when the number of tags exceeds small numbers, such as N>8, N>12, or N>16.

As noted, the single stranded tag sequences in a tag reporter assay may in principle be any distinct sequences that are compatible with the details of the assay, such as for example any sequences in the length range of approximately 10-100 bases that is compatible with PCR amplification, in the case of the assay of FIG. 34. However, when a set of N tags is used as a multiplex tag set as in FIG. 33, if any of the different tag sequences behave poorly in the pooled hybridization reaction used for the readout, either due to poor behavior of the individual tag, or cross hybridization between tags, the multiplex assay is negatively impacted in its sensitivity, specificity, or accuracy. Therefore, in preferred embodiments, the tags used for multiplexing are specifically designed as a set of N tags to be well-behaved in a specific hybridization reaction condition.

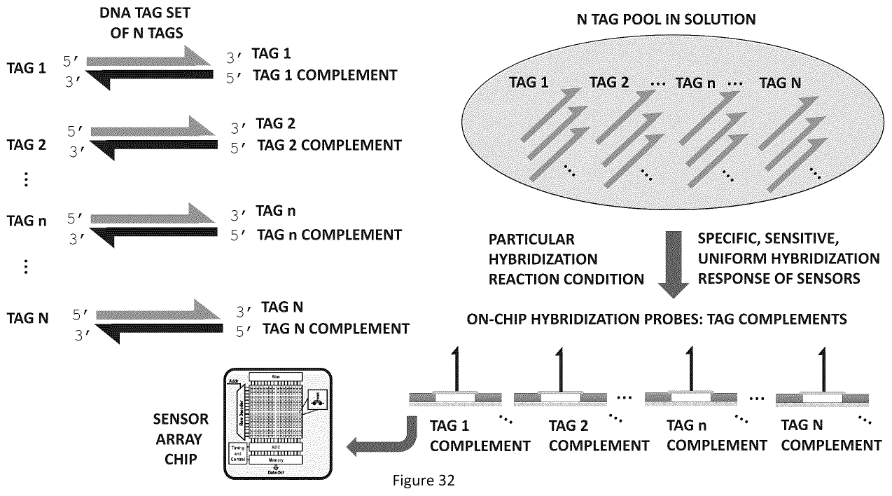

The concept of such an optimized DNA tag set is illustrated in more detail FIG. 32. There the set consists of N tags, which are distinct single stranded DNA oligos, each having its respective complementary oligo, referred to as the tag complement. When used in a multiplex tag assay, some subset of these tag oligos will be present in the output pool, in various concentrations. Therefore, the tag set as a whole, when pooled at some concentration, should desirably behave well when hybridized to the corresponding tag sensor array under a particular reaction condition, as defined by the such conditions including overall DNA concentration, temperature, buffer composition, duration and washing process. The sensor array comprises probes which have the tag complements as hybridization probes, in accord with any of the embodiments disclosed above. Such sensors are in preferred embodiments deployed on a sensor array chip. In preferred embodiments, each tag complement is represented on multiple pixels of the array, as indicated in FIG. 32, to provide redundant measurement capabilities.

In preferred embodiments of a tag set, the candidate tags for a tag set are designed theoretically to have desirable hybridization properties, using theoretical models for modelling hybridization interactions. In further preferred embodiments, such designed candidate tags and their complements are synthesized into physical DNA oligos, the corresponding tag complement arrays are produced, and then the tag set is experimentally screened under the desired candidate reaction conditions with the tag array chips, using an experimental design that can assess the different tag sensor performance properties of interest, which may include high sensitivity, high specificity, high signal to noise ratio, large dynamic range, linear response to concentration, and for low levels of undesirable cross hybridization or off target hybridization sensor responses, and for repeatable and consistent performance or low coefficient of variability across replicate experiments, and robust performance across the range of hybridization reaction conditions of interest. Based on such empirical screening, a subset of tags is selected that have good verified performance across all of synthesis, array fabrication, and hybridization sensor performance for multiplex tag assays.

In this way, tag sets that are well-designed and empirically verified for performance can be produced. In preferred embodiments, such a designed and verified tags set probes are synthesized in bulk with thorough application of well-known DNA synthesis quality controls, standard array chips are produced with probes for the tag set, and the resulting tag designs and tag arrays are used for diverse applications and classes of tag reported assays.

In preferred embodiments, the experimental design for empirical selection of a tag set may include hybridizing pools of tags that represent different subsets of the tags, different tag concentrations, and different hybridization reaction parameters, such as reaction temperature, buffer composition, and duration or washing conditions. Buffer composition may in particular include the salt ion concentration (such as Na+, or K+), the divalent cation concentration (such as Mg++), which have a strong effect on the melting point of hybridized DNA. In preferred embodiments, the tag set and hybridization reaction are co-optimized in this screening process, such that the outcome is an optimally performing tag set with a corresponding optimal hybridization reaction. In preferred embodiments, the screening will in particular identify tags that cross hybridize to off target sensors (i.e. those that do not have the tag complement), and will also identify tags that do not hybridize well to their target sensor. In preferred embodiments, the design will also identify the ideal or preferred reaction temperature and salt concentration, and divalent cation concentration, as well as the concentration of any well-known and common additives to hybridization reactions that may be used to control or modify hybridization stringency, alter melting point temperatures, or otherwise block unwanted interactions, such as betaine, DMSO, glycerol, Formamide, SDS, Tween-20, Triton-X, BSA, or blocking DNA, as well as other such well known and widely used additives.

In one preferred embodiment of the tag screening experimental design, each tag is individually hybridized to the array, at a representative range of concentrations, and under candidate reaction conditions, to directly check that it does not produce off target response, and that its on-target sensor has good response. Poor performing tags are then eliminated from the final set. This is not directly feasible when there are a large number of tags, such as N>100. In such a case, small subsets of tags could be similarly screened in pools, such as n tags at a time, n«N, e.g. n=10, tags with bad specific sensor performance are so identified for elimination, while if the pool shows off-target performance, each tag in that pool can be individually tested to determine which is the source of the off-target behavior, and in so doing identify the tags to be eliminated for off-target effects. More generally, even more efficient experimental designs can be used, which test for multiple effects within each pooled reaction. Many such informative experimental designs that test multiple factors within each pool would be obvious to those with expertise in DNA hybridization reactions and experimental design. The design of such experiments is also an area that is well known to those skilled in the statistical design of experiments (DOE), and such experimental designs can be produced with the help of well-known and widely used DOE analysis software, such as the JMP software package from the SAS corporation.

In preferred embodiments, the candidate DNA tag sequences for a tag set are designed to promote good performance as hybridization tags in tag reporter assays. Such preferred sequence design features may include sequences that promote efficient DNA synthesis, sequences that will have similarly hybridization responses-within some particular hybridization assay condition—in terms of uniformity across all tags of: sensitivity of sensor response, specificity of sensor response, dynamic range of sensor response, linearity of sensor response to concentration of the target tag, and absence of unwanted hybridization interactions that tend impair such performance, such as tags that have internal secondary structure (i.e. a single tag molecule folding onto itself), the tag complement having internal secondary structure (both of which would interfere with the desired tag target—tag probe hybridization), and unwanted hybridization of tags with the incorrect tag probe (off target binding to sensors), and unwanted tag vs tag solution phase hybridizations (which could reduce the amount of such tags available to bind with the respective tag sensors). In preferred embodiments, such sequence design specifications include matching the melting point, Tm, for all the tag-tag complement duplexes-relative to a given hybridization reaction condition (buffer composition, reaction temperature, tag concentration) —to be within a narrow range around a target melting point, and also include insuring that the melting point for tag self-interactions are much lower than Tm, and that the melting point for tag cross-hybridizations of tags with off-target tag probes having a much lower melting point than Tm. In preferred embodiments, such sequence designs will target the Tm to all be in an interval of [Tm₁, Tm₂], where in various preferred embodiments, the target tag melting point Tm₁ may be near 90° C., near 80° C., near 70° C., near 60° C., near 50° C., near 40° C., near 30° C., near 20° C., or near 10° C., and the range tag melting points for the set, Tm₂— Tm₁, is preferably less than 5° C., or preferably less than 2° C., or preferably less than 1° C. In preferred embodiments, sequences of the tag set are designed so that the melting points of all unwanted reactions will be much lower than the desired Tm range, by some threshold difference Delta. In particular, all tag self-interactions (and tag complement self-interactions) in the set will have melting points below Tm₁-Delta_{self}, and all cross-hybridization interactions tagᵢ vs tagⱼ probe, i≠j, (as well as tagᵢ vs tagⱼ interactions) will have melting point temperatures below Tm₁-Delta_{cross}. In preferred embodiments, these Delta temperature separations between the desired perfect match hybridization and unwanted hybridizations will be greater than 10° C., greater than 20° C., or greater than 30° C., or greater than 40° C. In preferred embodiments, that tag sequences in a tag set may all be of the same length, this may promote more convenient and efficient DNA synthesis, as well as promote uniformity of sensor performance, by reducing physically variability between sensors (such as, for example, the amount of molecular charge on the sensor).

In preferred embodiments, such tag sets may have up to 10 tags, up to 100 tags, up to 1000 tags, up to 10,000 tags, up to 1 million tags, or up to 10 million tags, or up to 100 million tags. For example, over 10 million SNP variants have been identified in the human genome, so a tag set with over 10 million tags would be needed to assay for all of these in a single assay. Of these, nearly 1,000,000 SNPS with clinical associations have been found, so a tag set of 1,000,000 tags would be appropriate for assaying for all of those. Over 10,000 SNPs have strong clinical indications have been for, so a tag set on this scale would be required to assay for all of those, and over 600 SNPs have clinical actions associated with them, so a tag set on the scale of 1000 tags would be required to assay for them. For another example, over 14 common sequence variant strains have been identified for the SARS-CoV-2 virus, therefore a tag set with up100 tags would be appropriate to assay for these strains.

The tables below show specific embodiments of tag set designs, for 16-32 tags. These are intended to illustrate various of the sequence design criteria disclosed above, and one process by which such tags can be designed. For these tag designs, the DINAMelt web-based software package was used to predict secondary hybridization structures, and their associated free energy, entropy and melting points (see Markham, N. R. & Zuker, M. (2005) DINAMelt web server for nucleic acid melting prediction. Nucleic Acids Res., 33, W577-W581). This is intended to illustrate the type of secondary structure melting point prediction software that can be used for such design efforts. This software performs secondary structure calculations for individual strands (self-interaction) as well as between strands (perfect match tag-tag complement or cross-hybridized). The hybridization reaction conditions that are allowed to be specified for these calculations consist of the reaction temperature, T, the molar concentration of sodium ions ([Na+]), the molar concentration of divalent magnesium ions ([Mg++]), and the molar concentration of the strands undergoing the interaction (CT). The calculations below were all done for reaction conditions of T=37° C., [Mg++]=0M, and CT=0.00001M, and for two different [Na+] concentrations, 0.1M and 1M, as indicated below. The tag sets were designed by starting from randomly generated n-mer oligo candidate tags, formed with equal 25% frequency of A, G, T, C, and starting from 1000-2000 such random oligos, selecting subsets that have their perfect match duplex hybridization Tm in a narrow target range within a few ° C. of mean Tm for all such oligos, and then further sub-selecting for such oligos which have their self-interaction Tm_{self} below this range by a desired Delta in the range of 20-40° C., and then further selecting for a subset that had Tm_{cross} for all cross-hybridizations of the tag against the complement of the other tags also lower than Tm by a similar Delta. Oligos were further eliminated if they contained a run of more than 3 G's in a row or more than 3 C's in a row (i.e. if they contain . . . GGGG . . . or . . . CCCC . . . ) in order to avoid the unwanted secondary structure of so-called “G tetrads” in the tag or its complement, which are another unwanted DNA secondary structure not necessarily predicted by tools such as DINAMelt, or reflected in the associated melting points from DINAMelt. Starting from 1000-2000 candidate tags, a final set of 16-20 tags were produced matching all this design specifications. The example sets below were chose to represent examples where the perfect match Tm are near various points of interest in range of 30-90° C., and where the unwanted interactions had Tm that were all lower by 20-40° C. from the perfect matches. Each tag oligo set has tags of the same length, and the length is the major variable that controls the Tm target of the set. The method illustrated here can be readily generalized to generate tag sets of any size N, by starting with larger random candidate sets. Other methods than random generation can be used to generate starting candidates for the tag set, such as starting with sequences strings that have a large Hamming Distance between them, or other methods of generating strings that have many mismatches between any two members of the set. Many such methods of generating initial sequence candidate sets are well known and obvious to those with expertise in algorithms for the analysis of text strings or in coding theory. The selection criteria here are also one meant to be illustrative, and not limiting. Other criteria for eliminating sequences with unwanted secondary structures or interactions are also obvious to those expert in DNA secondary structure analysis.

This example tag set is based on 10-mer tags. Table 1 shows the tag sequences, written 5′-3′, as well as the reverse complement sequences, written 5′-3′, i.e. the sequences of the tag complements or tag probes on the array. These have Tm near 50° C. or near 40° C., depending on the sodium concentration, while the undesirable structure melting points all lower by nearly 30° C. The specific critical melting point parameters that characterize the design criteria are as follows: at sodium ion concentration [Na+]=1.0 M in the hybridization reaction, the melting points of the tag duplexes are in the range of Tm=49.5-50.5° C., a range of 1° C., while the self and cross hybridization reactions all have Tm_{self}<20° C. and Tm_{cross}<20.5° C., which are at least Delta=29° C. below the perfect match melting point. At a 10-fold lower sodium ion concentration [Na+]=0.1 M in the hybridization reaction, the these temperatures are reduced by roughly 10° C.: specifically, at this condition, the melting points of the tag duplexes are in the range of Tm=39.0-40.1° C., while the self and cross hybridization reactions all have Tm_{self}<10° C. and Tm_{cross}<10° C., which are at least Delta=29° C. below the perfect match melting point. These parameters can be summarized as
[Na+]=0.0M: Tm 49.5-50.5° C., Tm_{self}<20° C., Tm_{cross}<20.5° C.
[Na+]=0.0M: Tm 39.0-40.1° C., Tm_{self}<10° C., Tm_{cross}<10° C.

TABLE 1
10-mer tag set, N = 16

| TAG | TAG COMPLEMENT |
|---|---|
| AAGCAGAACC | GGTTCTGCTT |
| AGGCCTACAT | ATGTAGGCCT |
| GGAATCCGTT | AACGGATTCC |
| GAGGTTTCGA | TCGAAACCTC |
| CACCTGAGAC | GTCTCAGGTG |
| CGGATGTCAA | TTGACATCCG |
| AGGATTGTGC | GCACAATCCT |
| CGTCACCTTT | AAAGGTGACG |
| CCTAACAGCC | GGCTGTTAGG |
| GAACTGCACA | TGTGCAGTTC |
| CACAACCACA | TGTGGTTGTG |
| CGGCTTAAGA | TCTTAAGCCG |
| GACACCGAAT | ATTCGGTGTC |
| TAGTACGTCG | CGACGTACTA |
| GCCTCCAAAT | ATTTGGAGGC |
| GGATCTGTCG | CGACAGATCC |

This example tag set is based on 14-mer tags, shown in Table 2. These have Tm near 60° C. or near 50° C., while undesirable structure melting points all lower by approximately 35° C. The specific critical melting point design parameters for the tag set are:
[Na+]=1.0M: Tm 60.0-62.0° C., T_{self}<24° C., Tm_{cross}<24° C.
[Na+]=0.1M: Tm 48.8-50.8° C., Tm_{self}<15° C., Tm_{cross}<14° C.

TABLE 2
14-mer tag set, N = 16

| TAG | TAG COMPLEMENT |
|---|---|
| TTGAGCTAAACAGG | CCTGTTTAGCTCAA |
| TCTCCAAACACATG | CATGTGTTTGGAGA |
| GTGACCTATTTCGC | GCGAAATAGGTCAC |
| TCGTCCAGATCTAA | TTAGATCTGGACGA |
| AGGTGGAAGATCTC | GAGATCTTCCACCT |
| TACTAGTCACTCCT | AGGAGTGACTAGTA |
| ACAAAAGGACACTC | GAGTGTCCTTTTGT |
| GCCGAGTCATATTC | GAATATGACTCGGC |
| TCTAACTTTCACCG | CGGTGAAAGTTAGA |
| AAGGATAGAGCACA | TGTGCTCTATCCTT |
| GAAGAGAAAGTCCG | CGGACTTTCTCTTC |
| CGATACTTTTTGCG | CGCAAAAAGTATCG |
| CCAAGGTTCTCATC | GATGAGAACCTTGG |
| CGCAGCTTAGATAA | TTATCTAAGCTGCG |
| GTGCGACTCTTATT | AATAAGAGTCGCAC |
| CGATTGCCTGTAAA | TTTACAGGCAATCG |

This example tag set is based on 20-mer tags, shown in Table 3. These have Tm near 70° C. or near 60° C., while undesirable structure melting points all lower by approximately 30° C. The specific critical melting point design parameters for the tag set are:
[Na+]=1.0M: Tm 72.6-73.4° C., T_{self}<37.1° C., Tm_{cross}<39.8° C.
[Na+]=0.1M: Tm 60.1-61.6° C., Tm_{self}<28.9° C., Tm_{cross}<29.3° C.

TABLE 3
20-mer tag set, N = 25

| TAG | TAG COMPLEMENT |
|---|---|
| ATATTGCCACGCTAAGTTGG | CCAACTTAGCGTGGCAATAT |
| GAGATTCCGAGCATTTCCTC | GAGGAAATGCTCGGAATCTC |
| TAATTGAGTAAGGCGAGAGG | CCTCTCGCCTTACTCAATTA |
| TGTAAGCCATATTGCCGGAT | ATCCGGCAATATGGCTTACA |
| TTCTGTTTGGATGCCTTGTT | AACAAGGCATCCAAACAGAA |
| CGCTATGATGACTACTCCGT | ACGGAGTAGTCATCATAGCG |
| AGTTGGATTGTAAGCAGCAG | CTGCTGCTTACAATCCAACT |
| TGGAAGGATTGTCGGTTAGA | TCTAACCGACAATCCTTCCA |
| AACGCAAAGGATACAAGGTT | AACCTTGTATCCTTTGCGTT |
| CAATAGTCGTGGTTCCTGTG | CACAGGAACCACGACTATTG |
| TTGTCAAATTGCAACTCGGT | ACCGAGTTGCAATTTGACAA |
| CCTGTTCAGTATTGCGTTGA | TCAACGCAATACTGAACAGG |
| TGTCGAAAGTCATTAGGCCT | AGGCCTAATGACTTTCGACA |
| GGACTGAGAACTTTAAGCGC | GCGCTTAAAGTTCTCAGTCC |
| TCTGTTCTTTCGCACACTAC | GTAGTGTGCGAAAGAACAGA |
| CCATGCGTGTTGTCTATGAC | GTCATAGACAACACGCATGG |
| CATGTACGCGGTATCAATGG | CCATTGATACCGCGTACATG |
| GACTACTTGCTCAATCTGCC | GGCAGATTGAGCAAGTAGTC |
| ACTGGTTAGGCGTATCTGAG | CTCAGATACGCCTAACCAGT |
| TATACGAAGCCTCACTGACA | TGTCAGTGAGGCTTCGTATA |
| TATCATGCTTGGACCTTTCT | AGAAAGGTCCAAGCATGATA |
| CGATCTCAATAACTCGCTGC | GCAGCGAGTTATTGAGATCG |
| GTTATGAGTTGTACCGCTGG | CCAGCGGTACAACTCATAAC |
| ACCTTAGGTCAGTCTTCCAT | ATGGAAGACTGACCTAAGGT |
| ACTGTGCACGACATAATTGG | CCAATTATGTCGTGCACAGT |

TABLE 4
30-mer tag set, N = 32

| TAG | TAG COMPLEMENT |
|---|---|
| AACTCCTTGGACATATGAGGCTAAGGCATA | TATGCCTTAGCCTCATATGTCCAAGGAGTT |
| GTAAAGTGAGACGCCGTGCAAGTTAATGGA | TCCATTAACTTGCACGGCGTCTCACTTTAC |
| ATGGCCACAAACTAATATTTGCCGAGCGAG | CTCGCTCGGCAAATATTAGTTTGTGGCCAT |
| AACTTAGGTCTCGGTTAAACGCCAACTTGA | TCAAGTTGGCGTTTAACCGAGACCTAAGTT |
| ACGTAACTGATAGGCGTTTGTACCACCATT | AATGGTGGTACAAACGCCTATCAGTTACGT |
| CTACATGAGATGGAGCGCGATTTTGCACAT | ATGTGCAAAATCGCGCTCCATCTCATGTAG |
| CGAAAGATTACCAGCCGCTATTAGACGACG | CGTCGTCTAATAGCGGCTGGTAATCTTTCG |
| TCCGTATCCTGATTGACTTCAGCATTAGCC | GGCTAATGCTGAAGTCAATCAGGATACGGA |
| GGACCACCTGTGTCACTATTACCATACGCT | AGCGTATGGTAATAGTGACACAGGTGGTCC |
| CTGACAGGTAGTAGTGCGTCATTGCTGAGG | CCTCAGCAATGACGCACTACTACCTGTCAG |
| AGAACGGTTAGGAGATTGAAGTACGAGCGC | GCGCTCGTACTTCAATCTCCTAACCGTTCT |
| AGTCACTTGTTCGAGTCAGTAGTCCGAGCA | TGCTCGGACTACTGACTCGAACAAGTGACT |
| GCGCTCCGTAAAGTAACGTCTCGATTATGC | GCATAATCGAGACGTTACTTTACGGAGCGC |
| GTCGTAATATTGGCTTACACATTGCGGCCA | TGGCCGCAATGTGTAAGCCAATATTACGAC |
| GCGGCAAACAGGTAATTTGGATAGCACAGT | ACTGTGCTATCCAAATTACCTGTTTGCCGC |
| TCTGTGGCAACGTCCATTTCTGTAGTACAC | GTGTACTACAGAAATGGACGTTGCCACAGA |
| GGTATTTAGTCTGCGATACCTTGAGCGTCG | CGACGCTCAAGGTATCGCAGACTAAATACC |
| GCCAGTCATTAGCCAGACCTTCAGAGACAA | TTGTCTCTGAAGGTCTGGCTAATGACTGGC |
| GTTCTCTGTACAATCGCAGCCACGAGTAGA | TCTACTCGTGGCTGCGATTGTACAGAGAAC |
| ATCTTGTCCGTGTCAAGTACTTGCACTAGC | GCTAGTGCAAGTACTTGACACGGACAAGAT |
| TTGTCCAATATCAACAGGCAGTCGAGTTCC | GGAACTCGACTGCCTGTTGATATTGGACAA |
| TGAACCAACAGCGTAATAGGACTAGGCCAC | GTGGCCTAGTCCTATTACGCTGTTGGTTCA |
| ACCAGAACCTTGCAACGGTGAGCATTTTAG | CTAAAATGCTCACCGTTGCAAGGTTCTGGT |
| AGTGGTCAGTTAGATCGAATGCACAGCTGT | ACAGCTGTGCATTCGATCTAACTGACCACT |
| CATCAATAGTGCAGCCTGTCATCCGATTGG | CCAATCGGATGACAGGCTGCACTATTGATG |
| TTCCAGGACCAAAGCCTAGGATGACACTTC | GAAGTGTCATCCTAGGCTTTGGTCCTGGAA |
| GACTTCTCACGTTTCGACATTCCGTCCACA | TGTGGACGGAATGTCGAAACGTGAGAAGTC |
| TATCTGCACACAACAGAGCATCCTCGTTAA | TTAACGAGGATGCTCTGTTGTGTGCAGATA |
| ACGGCCGTGATTCAATCTTTCGAGGAATTG | CAATTCCTCGAAAGATTGAATCACGGCCGT |
| TTCAGTTAGAACCGAGTTTGCGAACCGATA | TATCGGTTCGCAAACTCGGTTCTAACTGAA |
| ATGTATGCATGTTGAGTTCCATACGGCCTG | CAGGCCGTATGGAACTCAACATGCATACAT |
| CGTCCATGGTCAGTATACGCCACAGAATAC | GTATTCTGTGGCGTATACTGACCATGGACG |

This example tag set is based on 30-mer tags, shown in Table 4. These have Tm near 80° C. or near 70° C., while undesirable structure melting points all lower by approximately 30° C. The specific critical melting point design parameters for the tag set are:
[Na+]=1.0M: Tm 81.3-82.9° C., Tm_{self}<49.9° C., Tm_{cross}<49.7° C.
[Na+]=0.1M: Tm 68.7-70.4° C., Tm_{self}<47.7° C., Tm_{cross}<36.6° C.
Table 4. 30-mer tag set, N=32

This example tag set is based on 40-mer tags, shown in Table 5. These have Tm near 85° C. or near 75° C., while undesirable structure melting points all lower by approximately 30° C. The specific critical melting point design parameters for the tag set are:
[Na+]=1.0M: Tm 85.9-86.1° C., T_{self}<55.7° C., Tm_{cross}<49.1° C.
[Na+]=0.1M: Tm 72.9-73.7° C., T_{self}<53.1° C., Tm_{cross}<38.5° C.

TABLE 5
40-mer tag set, N = 16

| TAG | TAG COMPLEMENT |
|---|---|
| TCGCTTATGTGGATGCACTACTCCAGACCATTAGTGACAC | GTGTCACTAATGGTCTGGAGTAGTGCATCCACATAAGCGA |
| GTGAACCTATCGTTAGCAGTGTACCAGATCCGTCAGTGTA | TACACTGACGGATCTGGTACACTGCTAACGATAGGTTCAC |
| AAGATCGAAGCGTAGTGTACAAGGAGGTAGGCGAAAAACC | GGTTTTTCGCCTACCTCCTTGTACACTACGCTTCGATCTT |
| CGACGTTACCATGAACCTAAATCCATATGAGCGCTCCTGT | ACAGGAGCGCTCATATGGATTTAGGTTCATGGTAACGTCG |
| CAGGTCGGCTATGAGGTATTAATTGAGCGTGCTCACTCAG | CTGAGTGAGCACGCTCAATTAATACCTCATAGCCGACCTG |
| TTTCACTGTCAGATGTATCCTAGCATCCACTGCGCTACAC | GTGTAGCGCAGTGGATGCTAGGATACATCTGACAGTGAAA |
| TAGTTGACAGATGTTACTAGAGATGCGTGACGACGATGCG | CGCATCGTCGTCACGCATCTCTAGTAACATCTGTCAACTA |
| AACTCCTCCTAGACTCCATTGCAAAGTTGAGAACCTCGTA | TACGAGGTTCTCAACTTTGCAATGGAGTCTAGGAGGAGTT |
| AGTTGACAACTAGGCCGTTCCATGTTCACTCCAAAAAGCA | TGCTTTTTGGAGTGAACATGGAACGGCCTAGTTGTCAACT |
| GGAACAGTGTACGTCGACAATATGACAACGGATGCTCAGT | ACTGAGCATCCGTTGTCATATTGTCGACGTACACTGTTCC |
| GATTAGGCTAACAGACTGTAGGAAAGGCTCAGGATGGCTC | GAGCCATCCTGAGCCTTTCCTACAGTCTGTTAGCCTAATC |
| CCTCGAGTGCCAACAATAAGCGTTTTGATGCGGTTTTTTG | CAAAAAACCGCATCAAAACGCTTATTGTTGGCACTCGAGG |
| TGTACGTCCTTTCGTTATTTGCCGCTACTCACAACGTTGC | GCAACGTTGTGAGTAGCGGCAAATAACGAAAGGACGTACA |
| TGGTTGTTCATACACCAGAATGGTTTCAGTGATCAGCCGG | CCGGCTGATCACTGAAACCATTCTGGTGTATGAACAACCA |
| GACAGCCGTATAGATGTAAGATTTGGCCATGCGCACTTCA | TGAAGTGCGCATGGCCAAATCTTACATCTATACGGCTGTC |
| AGAGCCGCTCACATCTGTGAACTGATGTATACACGTCATA | TATGACGTGTATACATCAGTTCACAGATGTGAGCGGCTCT |

This example tag set is based on 50-mer tags, shown in Table 6 (reverse complement sequences are not shown, for brevity). These have Tm near 90° C. or near 76° C., while undesirable structure melting points all lower by approximately 30° C. The specific critical melting point design parameters for the tag set are:
[Na+]=1.0M: Tm 89.6-90.4° C., Tm_{self}<55.7° C., Tm_{cross}<54.9° C.
[Na+]=0.1M: Tm 76.5-77.7° C., Tm_{self}<53.1° C., Tm_{cross}<45.2° C.

TABLE 6
50-mer tag set, N = 16
TAG

GTCGATGTATCGAGATCAACACTTTCAAGCCGGCGACAACCTGATCAGCT
TACTAGAGTAATAGGCATTTGATGTTGCGCAACGATACGGCCGAACCAGC
CGGTGCCGACGTTCGCGTAAGCTATGCATGTATAGTCAAAACGACCGAAC
CCATCCTCTCCTAAGTCCGGTGGCCTCAATCCGTTCACCTATCGTAAACA
TATTATGCCTGTAGATATCTGCACACATGGTCAAGCACCGGCGTAACCAG
GACGACTGTTTCCATGGTGGTGACGCGTTAGATGAGAAGGTATTCTGCGT
AATTTCGCAGCAGAGACCATTTGAGCGCCAGTGACTTACAGAAAGCCAAT
CGACCATGTGCAGAAGGCGGTAAAAGCGAGACCATCTTAGTAGCTATCCG
ACAGTACTACGAGGCATGAAAACGAATTGGTATCTCAGGCGGAATCGCGC
AGGTATTGTCGCTGGTTCAGCTCGAAGTCGAAATGTCCGTACTCAATCGT
TCTAGTTAGGCGCGTGCTAGCAGGAGTAACAAAACGGTCGTAATGCAGAA
CCACACACGCTCCACAGGCCAGTTTATTACCGAACTTAGTGCAATTGCAG
AGTCAAGTTAAATGCCAAACCAGATCCTCGTCGCTGAAGGCATCAATCGA
CTTTACCTGGACCAGCATTTCCGTGTTCATAGAGTAACTCGTGGCCGTCT
GGAGTCCGACCTCCACTCAGTAACTGAACGTCAACGAGACACCGTTTAGC
GCAGGAACGATCGCGCGGATGCTCAATCATACTGAACCTTGTTATGCTTT
GCATCATTGTGCCGCTCTAAACCTAATGTCGTTGCACCATGTCTCGGAAC
GTTGTCAAGGACTGATGGCGGCCGTCGTTGCGTATCATCTTCGTAGATCA

This example tag set is based on 60-mer tags, shown in Table 7 (reverse complement sequences are not shown, for brevity). These have Tm near 90° C. or near 80° C., while undesirable structure melting points all lower by approximately 35° C. The specific critical melting point design parameters for the tag set are:
[Na+]=1.0M: Tm 91.6-92.4° C., Tm_{self}<54.7° C., Tm_{cross}<54.7° C.
[Na+]=0.1M: Tm 78.5-79.2° C., Tm_{self}<49.8° C., Tm_{cross}<42.5° C.

TABLE 7
60-mer tag set, N = 16
TAG

CAGAAACCAGTCAAGGCACAGTGAACGCCATAGGTTCCGTTTAGGCTAGTACGAGCTGTG
GAGTGTAGACAATGGCACGGCACTGAAAGTGAAATGGCCACTCCACTTAGAGAGCACCAA
GTCTTCAAATATGCATCAGGCAGCCGTCACTAATACGCGTACCTATGCCTCCATCACGGC
CACAATATAAGGACAACCATGCTCAGCAAAGCGGATAGTCGTGGCTCCAGTTTCCAGCGG
ACAACGCCAATCCAATGTGCACCGTACGCCAAATCAGCTACTAATAGATCAATGTCGGCG
AAGCTATCTGGCACGAAACCGTCCAGTAATCAGTGATCAGCACCACATAAGCTCACGGAG
CCTCGCGGACGTATCCTTCTTACTTCATGCCGGAGCCGTGTATTCTACATCCGTTGAAAG
ATACCAGATGTTCCTGGCTCTCAACACATAAACAGAACGCAACCAAGAGGCTCCGTCGTA
TAACATCAGAAGTCATAGGACCGAGCAAGGAAGGTGTGGCGTGCAAGTCGTTTTAGAGCC
TACTACAAGTGACCGGCGCGCGTTTAAGGTGTGACAATTACAGTGCGTGAAAAACTCTCC
TCCGCCTCCGCTAAGGAAAGCAAGCCAACCGGCTGTGTGAGAAGATTTTAGAAAGACTCA
GAAGAAAGTTTGTTTTGTTCGCGACAGCAATAAGCGGTTTACGCATCTTTCGGCCGGCAG
GCGCAGATCCGTCGATTACAGCTACTTCCGCCTTAGGCCGATACCTTTGTTGTTTCATCC
CCGTCCGAAACTGTCCACGTTAGTTACAACCACTAATTCTCTCTGCCTTCTCCTGGCCGT
TACCTGTTTGGCGAACCAGCCTTCTCTTCATGAGGTGTCACGTCTGCACTAGGAGAGTAG
GGCCTGTGTTGGCATCTTGAACTCAGCTCACGAGATAAGTTGCGGACTAGGTCTAATCGG

The design of sequences for bipartite tag sets, for use in assays such as that illustrated in FIG. 39, can be done by similar means, although the candidate tag sequences are created from all possible joins of candidate A ½ tags and candidate B ½ tags. To construct such a tag set, one preferred embodiment method is to start from ½ tags generated initially as larger sets of random n-mer sequences, and then the set of all such joined A:B tags can be assessed for their perfect match duplex Tm and cross-hybridization and self-hybridization Tm's, as above, and screened as described above to find a sub set of A and B half tags whose set of A:B join tags have similar Tm and substantially lower unwanted structure melting points. The result is a final set of M A ½ tags, and M B ½ tags, which imply the full N=M×M A:B tags. In addition, depending on the method of joining tags, there may be an insert sequence between the joins. These possibilities are illustrated here, based on the 10-mer half tags. A set of M=4 A ½ tags and M=4 B ½ tags are shown in Table 8. The resulting sets of N=4×4=16 A:B bipartite tags are shown in Tables 9-12, where the joins illustrated there have, respectively, no insert, a T insert, an AAAC insert, or a 10-base insert (ATTCGGTGTC). Many such methods of generating initial ½ sequence candidate sets are well known and obvious to those with expertise in algorithms for the analysis of text strings or in coding theory. Other criteria for eliminating sequences with unwanted secondary structures or interactions are also obvious to those expert in DNA secondary structure analysis.

Table 8 Shows the example A and B ½ tags, written 5′-3′, which are 10-mers in this illustration. These are used to create the A:B tag sets shown in subsequent tables.

TABLE 8
10-mer half tags for A:B bipartite tag formation

| A ½ TAG | B ½ TAG |
|---|---|
| CACAACCACA | GCACAATCCT |
| CCTAACAGCC | AAAGGTGACG |
| CGGCTTAAGA | CGACGTACTA |
| GGAATCCGTT | ATTTGGAGGC |

When these 10-mer ½ tags of Table 8 are joined with no insert, the resulting 20-mer bipartite tag set of 16 tags is shown in Table 9, along with the reverse complements of the tags. This 16-tag set of bipartite tags have Tm near 75° C. at a target sodium ion concentration of 1.0M, while undesirable structure melting points all lower by approximately 10° C. The specific critical melting point design parameters for the bipartite tag set are:
[Na+]=1.0M: Tm 73.1-77.6° C., Tm_{self}<67.3° C., Tm_{cross}<59.1° C.

TABLE 9
20-mer bipartite tag set, total N = 4 × 4 = 16,
with join insert of length 0

| A:B TAG 20-MERS | TAG COMPLEMENT |
|---|---|
| GGAATCCGTTGCACAATCCT | AGGATTGTGCAACGGATTCC |
| GGAATCCGTTAAAGGTGACG | CGTCACCTTTAACGGATTCC |
| GGAATCCGTTCGACGTACTA | TAGTACGTCGAACGGATTCC |
| GGAATCCGTTATTTGGAGGC | GCCTCCAAATAACGGATTCC |
| CCTAACAGCCGCACAATCCT | AGGATTGTGCGGCTGTTAGG |
| CCTAACAGCCAAAGGTGACG | CGTCACCTTTGGCTGTTAGG |
| CCTAACAGCCCGACGTACTA | TAGTACGTCGGGCTGTTAGG |
| CCTAACAGCCATTTGGAGGC | GCCTCCAAATGGCTGTTAGG |
| CACAACCACAGCACAATCCT | AGGATTGTGCTGTGGTTGTG |
| CACAACCACAAAAGGTGACG | CGTCACCTTTTGTGGTTGTG |
| CACAACCACACGACGTACTA | TAGTACGTCGTGTGGTTGTG |
| CACAACCACAATTTGGAGGC | GCCTCCAAATTGTGGTTGTG |
| CGGCTTAAGAGCACAATCCT | AGGATTGTGCTCTTAAGCCG |
| CGGCTTAAGAAAAGGTGACG | CGTCACCTTTTCTTAAGCCG |
| CGGCTTAAGACGACGTACTA | TAGTACGTCGTCTTAAGCCG |
| CGGCTTAAGAATTTGGAGGC | GCCTCCAAATTCTTAAGCCG |

When these 10-mer ½ tags of Table 8 are joined with a T insert, the resulting 21-mer bipartite tag set of 16 tags is shown in Table 10, along with the reverse complements of the tags, and with the join insert underlined. This 16-tag set of bipartite tags have Tm near 75° C. at a target sodium ion concentration of 1.0M, while undesirable structure melting points all lower by approximately 10° C. The specific critical melting point design parameters for the bipartite tag set are:
[Na+]=1.0M: Tm 73.0-77.3° C., Tm_{self}<62.9° C., Tm_{cross}<61.2° C.

TABLE 10
21-mer bipartite tag set, total N = 4 × 4 = 16,
with join insert of length 1

| A:B TAG 21-MERS | TAG COMPLEMENT |
|---|---|
| GGAATCCGTTTGCACAATCCT | AGGATTGTGCAAACGGATTCC |
| GGAATCCGTTTAAAGGTGACG | CGTCACCTTTAAACGGATTCC |
| GGAATCCGTTTCGACGTACTA | TAGTACGTCGAAACGGATTCC |
| GGAATCCGTTTATTTGGAGGC | GCCTCCAAATAAACGGATTCC |
| CCTAACAGCCTGCACAATCCT | AGGATTGTGCAGGCTGTTAGG |
| CCTAACAGCCTAAAGGTGACG | CGTCACCTTTAGGCTGTTAGG |
| CCTAACAGCCTCGACGTACTA | TAGTACGTCGAGGCTGTTAGG |
| CCTAACAGCCTATTTGGAGGC | GCCTCCAAATAGGCTGTTAGG |
| CACAACCACATGCACAATCCT | AGGATTGTGCATGTGGTTGTG |
| CACAACCACATAAAGGTGACG | CGTCACCTTTATGTGGTTGTG |
| CACAACCACATCGACGTACTA | TAGTACGTCGATGTGGTTGTG |
| CACAACCACATATTTGGAGGC | GCCTCCAAATATGTGGTTGTG |
| CGGCTTAAGATGCACAATCCT | AGGATTGTGCATCTTAAGCCG |
| CGGCTTAAGATAAAGGTGACG | CGTCACCTTTATCTTAAGCCG |
| CGGCTTAAGATCGACGTACTA | TAGTACGTCGATCTTAAGCCG |
| CGGCTTAAGATATTTGGAGGC | GCCTCCAAATATCTTAAGCCG |

When these 10-mer ½ tags of Table 8 are joined with an AAAC insert, the resulting 24-mer bipartite tag set of 16 tags is shown in Table 11, along with the reverse complements of the tags, and with the join insert underlined. This 16-tag set of bipartite tags have Tm near 80° C. at a target sodium ion concentration of 1.0M, while undesirable structure melting points all lower by approximately 10° C. The specific critical melting point design parameters for the bipartite tag set are:
[Na+]=1.0M: Tm 76.1-79.9° C., T_{self}<68.0° C., Tm_{cross}<69.2° C.

TABLE 11
24-mer bipartite tag set, total N = 4 × 4 = 16,
with join insert length of 4 bases (underlined)

| A:B TAG 24-MERS | TAG COMPLEMENT |
|---|---|
| GGAATCCGTTAAACGCACAATCCT | AGGATTGTGCGTTTAACGGATTCC |
| GGAATCCGTTAAACAAAGGTGACG | CGTCACCTTTGTTTAACGGATTCC |
| GGAATCCGTTAAACCGACGTACTA | TAGTACGTCGGTTTAACGGATTCC |
| GGAATCCGTTAAACATTTGGAGGC | GCCTCCAAATGTTTAACGGATTCC |
| CCTAACAGCCAAACGCACAATCCT | AGGATTGTGCGTTTGGCTGTTAGG |
| CCTAACAGCCAAACAAAGGTGACG | CGTCACCTTTGTTTGGCTGTTAGG |
| CCTAACAGCCAAACCGACGTACTA | TAGTACGTCGGTTTGGCTGTTAGG |
| CCTAACAGCCAAACATTTGGAGGC | GCCTCCAAATGTTTGGCTGTTAGG |
| CACAACCACAAAACGCACAATCCT | AGGATTGTGCGTTTTGTGGTTGTG |
| CACAACCACAAAACAAAGGTGACG | CGTCACCTTTGTTTTGTGGTTGTG |
| CACAACCACAAAACCGACGTACTA | TAGTACGTCGGTTTTGTGGTTGTG |
| CACAACCACAAAACATTTGGAGGC | GCCTCCAAATGTTTTGTGGTTGTG |
| CGGCTTAAGAAAACGCACAATCCT | AGGATTGTGCGTTTTCTTAAGCCG |
| CGGCTTAAGAAAACAAAGGTGACG | CGTCACCTTTGTTTTCTTAAGCCG |
| CGGCTTAAGAAAACCGACGTACTA | TAGTACGTCGGTTTTCTTAAGCCG |
| CGGCTTAAGAAAACATTTGGAGGC | GCCTCCAAATGTTTTCTTAAGCCG |

When these 10-mer ½ tags of Table 8 are joined with a 10-base insert, ATTCGGTGTC, the resulting 30-mer bipartite tag set of 16 tags is shown in Table 12, along with the reverse complements of the tags, and with the join insert underlined. This 16-tag set of bipartite tags have Tm near 85° C. at a target sodium ion concentration of 1.0M, while undesirable structure melting points all lower by at least approximately 5° C. The specific critical melting point design parameters for the bipartite tag set are:
[Na+]=1.0M: Tm 82.2-85.3° C., Tm_{self}<64.5° C., Tm_{cross}<80.4° C.

TABLE 12
30-mer bipartite tag set, total N = 4 × 4 =16,
with join insert length of 10 bases

| A:B TAG 30-MERS | TAG COMPLEMENT |
|---|---|
| GGAATCCGTTATTCGGTGTCGCACAATCCT | AGGATTGTGCGACACCGAATAACGGATTCC |
| GGAATCCGTTATTCGGTGTCAAAGGTGACG | CGTCACCTTTGACACCGAATAACGGATTCC |
| GGAATCCGTTATTCGGTGTCCGACGTACTA | TAGTACGTCGGACACCGAATAACGGATTCC |
| GGAATCCGTTATTCGGTGTCATTTGGAGGC | GCCTCCAAATGACACCGAATAACGGATTCC |
| CCTAACAGCCATTCGGTGTCGCACAATCCT | AGGATTGTGCGACACCGAATGGCTGTTAGG |
| CCTAACAGCCATTCGGTGTCAAAGGTGACG | CGTCACCTTTGACACCGAATGGCTGTTAGG |
| CCTAACAGCCATTCGGTGTCCGACGTACTA | TAGTACGTCGGACACCGAATGGCTGTTAGG |
| CCTAACAGCCATTCGGTGTCATTTGGAGGC | GCCTCCAAATGACACCGAATGGCTGTTAGG |
| CACAACCACAATTCGGTGTCGCACAATCCT | AGGATTGTGCGACACCGAATTGTGGTTGTG |
| CACAACCACAATTCGGTGTCAAAGGTGACG | CGTCACCTTTGACACCGAATTGTGGTTGTG |
| CACAACCACAATTCGGTGTCCGACGTACTA | TAGTACGTCGGACACCGAATTGTGGTTGTG |
| CACAACCACAATTCGGTGTCATTTGGAGGC | GCCTCCAAATGACACCGAATTGTGGTTGTG |
| CGGCTTAAGAATTCGGTGTCGCACAATCCT | AGGATTGTGCGACACCGAATTCTTAAGCCG |
| CGGCTTAAGAATTCGGTGTCAAAGGTGACG | CGTCACCTTTGACACCGAATTCTTAAGCCG |
| CGGCTTAAGAATTCGGTGTCCGACGTACTA | TAGTACGTCGGACACCGAATTCTTAAGCCG |
| CGGCTTAAGAATTCGGTGTCATTTGGAGGC | GCCTCCAAATGACACCGAATTCTTAAGCCG |

The above examples of tag set designs are illustrative, and not meant to be limiting. From these examples, and the procedures disclosed, there are many variations on the design methodology disclosed that would be obvious to those expert in DNA secondary structure calculations and hybridization assay principles, that can similarly be used to design tag sets of arbitrary number of tags, and that are uniformly matched in their perfect duplex hybridization properties, and which have greatly reduced potential for all unwanted hybridization reactions within the set, including self-interactions, or cross-interactions, involving the tags or tag complements. Such method variations may include not just matching of melting points, Tm, but also matching of other thermodynamic parameters of tag duplexes, such as the entropy differential, enthalpy differential, or free energy differential. Such method variations also may also include consideration of an extended set of secondary structures that may occur for any given strand or pair of interacting strands, and estimating the effects of these multiple possible structures on the hybridization interactions within the tag set.

Tags Using Modified Nucleotides or Other Modifications. Another advantage of using the reporter tag framework, over direct detection of target native DNA, or other kinds of native targets, is that both the reporter tags and/or their complementary probes on the sensor array may make use of modifications which may be made to DNA oligos that could enhance the performance of the hybridization sensor, such as providing greater signal, greater signal to noise, or less cross-hybridization. Such modifications in preferred embodiments may include nucleotide analogues, modified bases, or signal enhancing labels. In preferred embodiments, this may include the use of Locked Nucleic Acids (LNAs) or Peptide Nucleic Acids (PNAs), or other modified forms of nucleic acids (XNAs). In some preferred embodiments, as disclosed, the reporter tag is generated by a polymerase replication of the tag complement, and not all such modifications can be specifically propagated through such a process. For such modifications that do not propagate under polymerase copying, the primary modifications must be on the tag probe on the sensor array. In this case, the tag set can still enjoy enhanced benefits of these modifications, versus their use with native DNA targets, because, in preferred embodiments, the tag sequences can be designed to get maximal performance benefit of such modifications residing in the array tag probes. This includes using the base pairing that are most impacted by these modifications (e.g. which specific base-vs-analog-base complements do the most to increasing desired proper pair Tm, or decrease unwanted mismatched pair Tm). Taking maximum advantage of these requires the tag design process outline above to may use of such rules, or in preferred embodiment, of their impact on the Tm calculations. Other modifications can be propagated through polymerase copying reactions, included extended genetic code analogs, and such modifications could therefore reside in the reporter tags themselves. Also, tags produced by ligation of tag parts may also readily include all such modifications under consideration.

Such signal enhancing groups that may be added to tags include in preferred embodiments, the use of biotinylated nucleotides (dNTPs) in the tag production assays that involve polymerase-based copying, such that resulting tags so produced are biotinylated via the presence of biotinylated nucleotides. As is well known to those expert in nucleic acid manipulation, this can be used for either subsequent tag purification processes to provide a purer pool of tags for better detection behavior on the sensor array, or this can be used for subsequent tag labeling by the biotin-avid conjugation reaction, to add avidin-based groups to enhance the sensor signals. As is well known to those expert in nucleic acid manipulation, other forms of conjugatable nucleotides (dNTPs) that are compatible with polymerase extension can be used, such as those with click chemistry groups, or any of many other known bioconjugation groups, biotin-avidin being just one well known and widely used exemplar.

Many such modifications are well-known to those skilled in nucleic acid chemistry, which have known properties of increase the melting point Tm of duplexes over corresponding native forms, when used in one or both strands, or which can also reduce the melting point/further destabilize the unwanted cross-hybridizations between tags, by increasing the energy costs of mismatched bases beyond the native levels. By using such modifications, the reporter tags can have much better hybridization properties as a set, relative to the detection sensor array, as compared to sets of native DNA reporter tags and tag probes, or also compared to native DNA hybridization target sets, such as natively occurring sequence segments of interest.

Methods and Applications for Infectious Disease In preferred embodiments, such testing or monitoring applications referred to above include testing for the presence of pathogens, and in preferred embodiments, testing for parasites, fungi, bacterial pathogens or viral pathogens. Such parasites include Malaria, Giardia, and Toxoplasmosis. Such bacterial pathogens include Salmonella, E. Coli.. Such viral pathogens include influenza, flu viruses, cold viruses-including rhinovirus, adenovirus, and human corona virus, HIV, Ebola, Dengue, Hanta, Zika and West Nile viruses, SARS, MERS, and COVID-19 virus, and novel viruses of DNA or RNA type related to or unrelated to these, that have a known genetic sequence to provide for defining relevant tag reporter assays such as those described in FIGS. 32 through 37, to identify such pathogens, or strains of such pathogens.

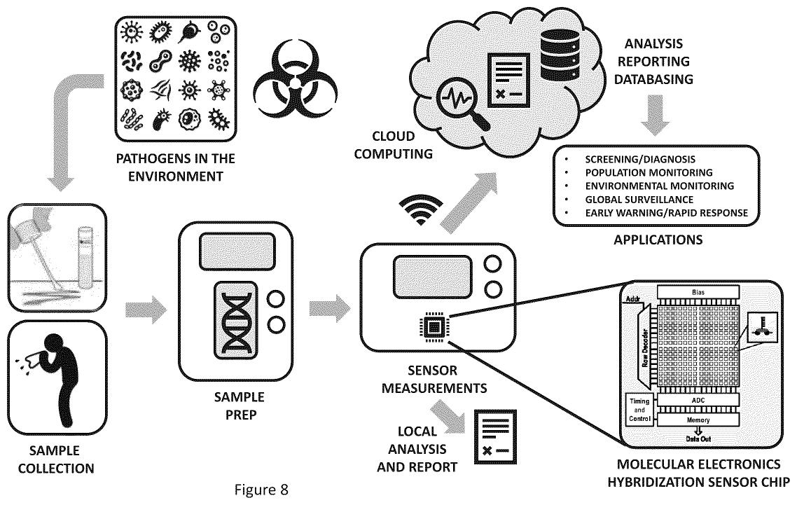

The major elements of preferred embodiments for infectious disease pathogen detection applications are disclosed here, as illustrated in FIG. 8. As indicated there, Infectious disease pathogens exist in the environment, and in preferred embodiments these are from among parasites, fungi, bacteria or viruses. A primary biosample is obtained, which in preferred embodiments may be material from a human subject, or a swab or other material collection from the environment, including possibly from plants or animals in the environment. This primary biosample is provided to a sample preparation system, which extracts and purifies DNA contained within the primary sample, and puts it into a form suitable for a tag reporter assay, and application of the resulting tag pool to the tag array sensor chip device disclosed herein. This sample is provided to an instrument that applies the sample to the chip, and controls the chip operations, and collects the sensor signal data from the chip. This instrument in preferred embodiments may locally analysis the data, record it locally, and produce a report on the pathogen content of the sample, such as on a screen on the instrument. In other preferred embodiments, this instrument transmits the data to a remote cloud, where analysis, reporting out on the pathogen content of the sample, and databasing of results can occur. Such cloud-enabled embodiments, in conjunction with many deployed instruments, are well suited to large scale efforts, such as national or international or global scale screening of samples for pathogens, for diagnosis of disease caused by pathogens, for monitoring the occurrence or spread of such disease at the population scale, or for monitoring such pathogens in the environment, and for global surveillance and early warning/rapid response efforts to contend with outbreaks of such infectious diseases.

In preferred embodiments for a testing methods and applications, a primary biosample is acquired directly from a test subject or the environment, and then some form of sample prep is required to prepare materials to the proper state to apply to the sensor device for measurement. The primary sample in preferred embodiments could be tissue, saliva, mucous, buccal swab, blood, sweat, urine, stool, out bodily fluids, or exhaled air, or material filtered from air or water, or material swabbed from a surface. It could also be such samples acquired from plants or animals in the environment, or from food, or from known vectors in the environment that carry such pathogens, such as bats, rodents, mosquitoes or snails. The sample prep could in preferred embodiments be a crude cell lysate extract containing DNA, or could be DNA further purified from the sample by standard purification column or filter paper purifications, or other extraction such as phenyl-chloroform. In preferred embodiments the purified sample could be the results from applying any of the many forms of PCR amplification reaction to the sample, which could in preferred embodiments be thermocycling or isothermal forms of PCR. In preferred embodiments, such sample prep is done by a self-contained sample prep device, or in other preferred embodiments, such a device integrated with the sensor platform, such as in the case of fully integrated point-of-us testing devices.

In preferred embodiments for the testing method using such devices and systems, the test system is deployed at a testing site, a primary biosample is collected and delivered to the testing site, to be tested for presence of a given pathogen or pathogen strain, a sample preparation process is applied to the primary sample to produce a product suitable for a tag reporter assay, and the resulting tag pool is to be applied to the molecular electronic sensor tag array chip device, which comprises a multiplicity of hybridization probe sensors that correspond to the tag set in use, and the device signals are readout, undergo primary local signal processing, and these data are then transferred to a centralized or cloud-based server for subsequent additional analysis or testing outcome report generation.

In preferred embodiments, the testing site could be a centralized testing facility of high capacity, for a business, hospital or other organization, or for a region such as a city, county, state, our country. In other preferred embodiments, the testing site could be a field deployment site, or a point-of-contact site, such as at an airport, transportation hub, or major gathering site such as an arena or stadium, or at an immigration control checkpoint or temporary monitoring point set up by the military, police, or government officials. In other embodiments, the testing site could be a mobile van that is deployed to sites as needed. In other preferred embodiments, the testing site may be in the home for private individuals. In other preferred embodiments, the testing site could be autonomous environmental monitoring stations deployed into the field, stationary or mobile, including driving, flying or aquatic drones, that monitor samples acquired locally from the environment, such as through filtering of air, or water, or trapping of known disease vectors or carriers in the environment, such as insects, rodents, bats or birds, or aquatic snails. In preferred embodiments, mosquitoes are one such vector.

In preferred embodiments, the primary biosample could be obtained as a swab of a surface that collects material deposited on the surface, as filtered material collected from air or water, or a water sample, or as a bodily fluid sample or buccal swab or saliva or excrement or tissue sample provided from a person or animal, or as a sample of a food item, or agricultural product.

In preferred embodiments, the sample collection may be done in close proximity to the test system, such as within 1 foot, 10 feet, or 100 feet, and in preferred embodiments such samples are rapidly delivered to the test system, such as within 10 seconds, one minute, 10 minutes or 1 hour, in order to have the benefit of distributed sample collection combined with rapid testing and test results. In preferred embodiments, the sample collection includes the assignment of a unique ID to the sample, such as an alpha-numeric code, serial number, barcode or QR code, to be used for sample tracking, and affiliation of final report back to the sample. In preferred embodiments, other identifying information may be collected and attached to the sample or affiliated with the sample ID, such as personal identifier, such as a personal name, social security number, government issued ID number, employee number, or date of birth, facial image or fingerprint.

In preferred embodiments, the sample preparation process comprises a PCR-based amplification method applied to the sample to produce amplified DNA material for detection. In other preferred embodiments, the sample preparation process is a process to extract and purify DNA or RNA without any amplification to produced purified material for detection. In preferred embodiment, this sample prep process is performed in a separate instrument from the sensor chip instrument, and is transferred to that instrument. In other preferred embodiments, the sample prep processes are performed on a subsystem integrated into the same instrument that runs the sensor chip device.

In preferred embodiments, the tag report assay may be performed off the sensor chip, the resulting tag pool may be applied to the tag array in either a purified form, that purifies for the released tags, or in unpurified form as produced by the tag reporter assay. In preferred embodiments, this tag reporter reaction may be done on a separate instrument, or integrated into the same instrument that runs the tag array sensor chip. In other preferred embodiments, the tag reporter assay may be performed in the flow cell or reaction volume of the tag array chip itself.

In preferred embodiments, the pathogen of interest is a pathogenic bacterium, such as E. Coli, or Salmonella, or Listeria, and the corresponding tag reporter assay probes include specific DNA probes that target segments common to many strains of such bacteria of interest, such as in FIG. 34 or 36. In other preferred embodiments, the pathogen is interest includes the specific strains of such bacteria, and the corresponding tag reporter assay probes include strain-specific DNA probes, such as in FIG. 35 or 37.

Application to Viral Pandemics and COVID-19 In other preferred embodiments, the pathogen of interest is a virus, such as influenza, flu viruses, cold viruses-including rhinovirus, adenovirus, and human corona virus, HIV, Ebola, SARS, MERS, and COVID-19, and novel viruses of DNA or RNA type related to or unrelated to these, that have a known genetic sequence to provide for defining tag reporter probes such as in the assays of FIG. 34 through 39. In preferred embodiments, the tag reporter probes include specific DNA probes common to many strains of such a virus of interest, such as in FIG. 34 or 36. In other preferred embodiments, the pathogen includes the specific strains of such viruses, and the corresponding tag reporter probes include strain-specific DNA probes, such as in FIG. 35 or 37.

TABLE 13
nucleic acid sequence of gene S from COVID-19. This sequence is used
to design gRNA sequences in certain preferred embodiments.

Gene S

| | | |
|---|---|---|
| | 1 | atgtttgttt ttcttgtttt attgccacta gtctctagtc agtgtgttaa tcttacaacc |
| | 61 | agaactcaat taccccctgc atacactaat totttcacac gtggtgttta ttaccctgac |
| | 121 | aaagttttca gatcctcagt tttacattca actcaggact tgttcttacc tttcttttcc |
| | 181 | aatgttactt ggttccatgc tatacatgtc tctgggacca atggtactaa gaggtttgat |
| | 241 | aaccctgtcc taccatttaa tgatggtgtt tattttgctt ccactgagaa gtctaacata |
| | 301 | ataagaggct ggatttttgg tactacttta gattcgaaga cccagtccct acttattgtt |
| | 361 | aataacgcta ctaatgttgt tattaaagtc tgtgaatttc aattttgtaa tgatccattt |
| | 421 | ttgggtgttt attaccacaa aaacaacaaa agttggatgg aaagtgagtt cagagtttat |
| | 481 | tctagtgcga ataattgcac ttttgaatat gtctctcagc cttttcttat ggaccttgaa |
| | 541 | ggaaaacagg gtaatttcaa aaatcttagg gaatttgtgt ttaagaatat tgatggttat |
| | 601 | tttaaaatat attctaagca cacgcctatt aatttagtgc gtgatctccc tcagggtttt |
| | 661 | tcggctttag aaccattggt agatttgcca ataggtatta acatcactag gtttcaaact |
| 1415 | 721 | ttacttgctt tacatagaag ttatttgact cctggtgatt cttcttcagg ttggacagct |
| | 781 | ggtgctgcag cttattatgt gggttatctt caacctagga cttttctatt aaaatataat |
| | 841 | gaaaatggaa ccattacaga tgctgtagac tgtgcacttg accctctctc agaaacaaag |
| 1428 | 901 | tgtacgttga aatccttcac tgtagaaaaa ggaatctatc aaacttctaa ctttagagtc |
| | 961 | caaccaacag aatctattgt tagatttcct aatattacaa acttgtgccc ttttggtgaa |
| | 1021 | gtttttaacg ccaccagatt tgcatctgtt tatgcttgga acaggaagag aatcagcaac |
| | 1081 | tgtgttgctg attattctgt cctatataat tccgcatcat tttccacttt taagtgttat |
| | 1141 | ggagtgtctc ctactaaatt aaatgatctc tgctttacta atgtctatgc agattcattt |
| | 1201 | gtaattagag gtgatgaagt cagacaaatc gctccagggc aaactggaaa gattgctgat |
| | 1261 | tataattata aattaccaga tgattttaca ggctgcgtta tagcttggaa ttctaacaat |
| | 1321 | cttgattcta aggttggtgg taattataat tacctgtata gattgtttag gaagtctaat |
| | 1381 | ctcaaacctt ttgagagaga tatttcaact gaaatctatc aggccggtag cacaccttgt |
| | 1441 | aatggtgttg aaggttttaa ttgttacttt cctttacaat catatggttt ccaacccact |
| | 1501 | aatggtgttg gttaccaacc atacagagta gtagtacttt cttttgaact tctacatgca |
| | 1561 | ccagcaactg tttgtggacc taaaaagtct actaatttgg ttaaaaacaa atgtgtcaat |
| | 1621 | ttcaacttca atggtttaac aggcacaggt gttcttactg agtctaacaa aaagtttctg |
| | 1681 | cctttccaac aatttggcag agacattgct gacactactg atgctgtccg tgatccacag |
| | 1741 | acacttgaga ttcttgacat tacaccatgt tcttttggtg gtgtcagtgt tataacacca |
| | 1801 | ggaacaaata cttctaacca ggttgctgtt ctttatcagg atgttaactg cacagaagtc |
| | 1861 | cctgttgcta ttcatgcaga tcaacttact cctacttggc gtgtttattc tacaggttct |
| | 1921 | aatgtttttc aaacacgtgc aggctgttta ataggggctg aacatgtcaa caactcatat |
| | 1981 | gagtgtgaca tacccattgg tgcaggtata tgcgctagtt atcagactca gactaattct |
| | 2041 | cctcggcggg cacgtagtgt agctagtcaa tccatcattg cctacactat gtcacttggt |
| | 2101 | gcagaaaatt cagttgctta ctctaataac tctattgcca tacccacaaa ttttactatt |
| | 2161 | agtgttacca cagaaattct accagtgtct atgaccaaga catcagtaga ttgtacaatg |
| | 2221 | tacatttgtg gtgattcaac tgaatgcagc aatcttttgt tgcaatatgg cagtttttgt |
| | 2281 | acacaattaa accgtgcttt aactggaata gctgttgaac aagacaaaaa cacccaagaa |
| | 2341 | gtttttgcac aagtcaaaca aatttacaaa acaccaccaa ttaaagattt tggtggtttt |
| | 2401 | aatttttcac aaatattacc agatccatca aaaccaagca agaggtcatt tattgaagat |
| | 2461 | ctacttttca acaaagtgac acttgcagat gctggcttca tcaaacaata tggtgattgc |
| | 2521 | cttggtgata ttgctgctag agacctcatt tgtgcacaaa agtttaacgg ccttactgtt |
| | 2581 | ttgccacctt tgctcacaga tgaaatgatt gctcaataca cttctgcact gttagcgggt |
| | 2641 | acaatcactt ctggttggac ctttggtgca ggtgctgcat tacaaatacc atttgctatg |
| | 2701 | caaatggctt ataggtttaa tggtattgga gttacacaga atgttctcta tgagaaccaa |
| | 2761 | aaattgattg ccaaccaatt taatagtgct attggcaaaa ttcaagactc actttcttcc |
| | 2821 | acagcaagtg cacttggaaa acttcaagat gtggtcaacc aaaatgcaca agctttaaac |
| | 2881 | acgcttgtta aacaacttag ctccaatttt ggtgcaattt caagtgtttt aaatgatatc |
| | 2941 | ctttcacgtc ttgacaaagt tgaggctgaa gtgcaaattg ataggttgat cacaggcaga |
| | 3001 | cttcaaagtt tgcagacata tgtgactcaa caattaatta gagctgcaga aatcagagct |
| | 3061 | tctgctaatc ttgctgctac taaaatgtca gagtgtgtac ttggacaatc aaaaagagtt |
| | 3121 | gatttttgtg gaaagggcta tcatcttatg tccttccctc agtcagcacc tcatggtgta |
| | 3181 | gtcttcttgc atgtgactta tgtccctgca caagaaaaga acttcacaac tgctcctgcc |
| | 3241 | atttgtcatg atggaaaagc acactttcct cgtgaaggtg tctttgtttc aaatggcaca |
| | 3301 | cactggtttg taacacaaag gaatttttat gaaccacaaa tcattactac agacaacaca |
| | 3361 | tttgtgtctg gtaactgtga tgttgtaata ggaattgtca acaacacagt ttatgatcct |
| | 3421 | ttgcaacctg aattagactc attcaaggag gagttagata aatattttaa gaatcataca |
| | 3481 | tcaccagatg ttgatttagg tgacatctct ggcattaatg cttcagttgt aaacattcaa |
| | 3541 | aaagaaattg accgcctcaa tgaggttgcc aagaatttaa atgaatctct catcgatctc |
| | 3601 | caagaacttg gaaagtatga gcagtatata aaatggccat ggtacatttg gctaggtttt |
| | 3661 | atagctggct tgattgccat agtaatggtg acaattatgc tttgctgtat gaccagttgc |
| | 3721 | tgtagttgtc tcaagggctg ttgttcttgt ggatcctgct gcaaatttga tgaagacgac |
| | 3781 | tctgagccag tgctcaaagg agtcaaatta cattacacat aa |

A double stranded nucleic acid sequence from COVID-19 used in embodiments herein is provided below.

5′-AACTTCTAACTTTAGAGTCCAACCAACAGAA [symbol] ATTGTTAGATAT
CCTA
3′-TTGAAGATTGAAATCTCAGGTTGGTTGTCTTAGATAACAATCTATA
GGAT

An exemplary gRNA coupled to a bridge is provided below (1428 Int-RNA).

TAATTTCTACTC/iAzideN/TGTAGAT GAGTC CAACC AACAG
AATCT

In certain embodiments, a nucleic acid probe comprises a nucleic acid sequence corresponding to or complementary a selected portion of a nucleic acid sequence of a pathogen of interest described herein. In such embodiments, a nucleic acid probe may for example comprise a nucleic acid sequence of at least 3, 4, 5, 6, 7, 8, 9, 10, 11, 12, 13, 14, 15, 16, 17, 18, 19, 20, 21, 22, 23, 24, 25, 30, 35, 40, 45 or 50 contiguous nucleotides of a pathogen of interest described herein. In alternative embodiments, a nucleic acid probe may comprise a nucleic acid sequence of 3, 4, 5, 6, 7, 8, 9, 10, 11, 12, 13, 14, 15, 16, 17, 18, 19, 20, 21, 22, 23, 24, 25, 30 to 35, 35 to 40, 40 to 45, or 45 to 50 contiguous nucleotides of a pathogen of interest described herein. In alternative embodiments, a nucleic acid probe may comprise any range of consecutive nucleic acid sequence numbering between 4, 5, 6, 7, 8, 9, 10, 11, 12, 13, 14, 15, 16, 17, 18, 19, 20, 21, 22, 23, 24, and 25, such as a range between 4 and about 18 contiguous nucleotides of a pathogen, between 5 and about 15 contiguous nucleotides of a pathogen, between 6 and 12 contiguous nucleotides of a pathogen, etc.

In other embodiments, the pathogen of interest is a virus selected from the group consisting of Adeno-Associated Virus, Adenovirus, Arena virus (Lassa virus), Alpha virus, Astrovirus, Bacille Calmette-Guerin ‘BCG’, BK virus (including associated with kidney transplant patients), Papovavirus, Bunyavirus, Burkett's Lymphoma (Herpes), Calicivirus, California, encephalitis (Bunyavirus), Colorado tick fever (Reovirus), Corona virus, Coronavirus, Coxsackie, Coxsackie virus A, B (Enterovirus), Crimea-Congo hemorrhagic fever (Bunyavirus), Cytomegalovirus, Cytomegaly, Dengue (Flavivirus), Diptheria (bacteria), Ebola, Ebola/Marburg hemorrhagic fever (Filoviruses), Epstein-Barr Virus ‘EBV’, Echovirus, Enterovirus, Eastern equine encephalitis ‘EEE’, Togaviruses, Encephalitis, Enterovirus, Flavi virus, Hantavirus, Bunyavirus, Hepatitis A., (Enterovirus), Hepatitis B virus (Hepadnavirus), Hepatitis C (Flavivirus), Hepatitis E (Calicivirus), Herpes, Herpes Varicella-Zoster virus, HIV Human Immunodeficiency Virus (Retrovirus), HIV-AIDS (Retrovirus), Human Papilloma Virus ‘HPV’, Cervical cancer (Papovavirus), HSV 1 Herpes Simplex I, HSV 2 Herpes Simplex II, HTLV-T-cell leukemia (Retrovirus), Influenza (Orthomyxovirus), Japanese encephalitis (Flavivirus), Kaposi's Sarcoma associated herpes virus KSHV (Herpes HHV 8), Kyusaki, Lassa Virus, Lentivirus, Lymphocytic Choriomeningitis Virus LCMV (Arenavirus), Measles (Rubella), Measels, Measles Micro (Paramyxovirus), Monkey Bites (Herpes strain HHV 7), Mononucleosis (Herpes), Morbilli, Mumps (Paramyxovirus), Newcastle's diseases virus, Norovirus, Norwalk virus (Calicivirus), Orthomyxoviruses (Influenza virus A, B, C), Papillomavirus (warts), Papova (M.S.), Papovavirus (JC-progressive multifocal leukoencephalopathy in HIV) (Papovavirus), Parainfluenza Nonsegmented (Paramyxovirus), Paramyxovirus, Parvovirus (B19 virusaplastic crises in sickle cell disease), Picoma virus, Pertussus (bacteria), Polio (Enterovirus), Poxvirus (Smallpox), Prions, Rabies (Rhabdovirus), Reovirus, Retrovirus, Rhabdovirus (Rabies), Rhinovirus, Roseola (Herpes HHV 6), Rotavirus, Respiratory SyncitialVirus (Paramyxovirus), Rubella (Togaviruses), Bunyavirus, Flavivirus, Poxvirus, Vaccinia virus, Variola, Venezuelan Equine Encephalitis ‘VEE’ (Togaviruses), Wart virus (Papillomavirus), Western Equine Encephalitis “WEE’ (Togaviruses), West Nile Virus (Flavivirus), and Yellow fever (Flavivirus).

In preferred embodiments, the primary data analysis performed on system includes data reduction algorithms that reduce the amount of data needed to be transferred off-system. Such methods may include discarding uninformative portions of the signal trace, subsampling or parameterization of parts of the signal trace, and general data compression algorithms known to those skilled in data compression, such as methods utilized in zip, gzip, bzip, and other common compression utilities. In preferred embodiments, the primary analysis also includes analysis of traces to produce a net hybridization intensity score for each probe on the sensor chip, and in preferred embodiments, a final call of detection, non-detection, or indeterminate measurement for each probe on the sensor chip. In other preferred embodiments, such analysis is done in the off-instrument phase of an analysis. In other preferred embodiments, the off-instrument analysis includes the generation of a final report that affiliates sample identifiers with the outcome of the test for the presence of pathogens of interest. Such identifies may include a subject name or assigned ID or other identifier provided at the point of sample collection, as well as sample identifiers such as the time and place of sample collection, and time and place of sample processing on the sensor chip system.

In preferred embodiments, the test is performed rapidly, with the time from providing the primary biosample, to completion of analysis and report generation being less than 24 hours, and in preferred embodiments, less than 8 hours, less than 4 hours, less than 1 hour, less than 30 minutes, or less than 15 minutes.

In a preferred embodiment, the system disclosed above is applied to the monitoring of the pandemic disease COVID-19, a viral disease outbreak in 2019 originating in Wuhan, China. In this application, the hybridization probes are selected to be complements to segments from the genome of the underlying virus, the Severe Acute Respiratory Syndrome Coronavirus 2, also designated SARS-CoV-2. This SARS-CoV-2 virus has a single stranded RNA genome, of size approximately 30,000 bases. One exemplar sequence for this genome is available at the Genbank® database as accession ID LC528232 (see https://www.ncbi.nlm.nih.gov/genbank/). Thus, in preferred embodiments where tag reporter assay directly detects the genomic material by hybridization, this will be DNA-RNA assay, and the sample prep must extract and purify RNA from the primary biosample. In preferred embodiments where the sample prep comprises a PCR amplification of the genome, this would be a reverse-transcriptase mediated PCR that produces amplified DNA product, either of specific target segments, or non-specific segments of the entire genome, and the resulting tag reporter assay is a DNA-DNA assay. By taxonomy, this virus a specific strain of the Severe Acute Respiratory Syndrome-related Coronavirus (SARSr-Cov), which is a species of coronavirus that infects humans, bats and certain other mammals. There are hundreds of known strains of this virus, and hybridization probes must be chosen for sequence segments that distinguish the COVID-19 strain from other harmless strains, or other disease-causing strains, such as the strain designated SARS-CoV, which caused SARS disease outbreak in 2002 in Guangdong Province, China. There are numerous sequence differences between these strains, providing many candidates for distinguishing target sequences. In certain preferred embodiments, a nucleic acid probe comprises a nucleic acid sequence corresponding to or complementary a selected portion of the entire nucleic acid sequence of a COVID-19 virus described herein. In such embodiments, a nucleic acid probe may for example comprise a nucleic acid sequence of at least 4, 5, 6, 7, 8, 9, 10, 11, 12, 13, 14, 15, 16, 17, 18, 19, 20, 21, 22, 23, 24, 25, 30, 35, 40, 45 or 50 contiguous nucleotides of a Coronavirus or Coronavirus-related virus of interest described herein.

In some preferred embodiments for COVID-19 testing, primary samples would be environmental surface swabs, or air filters, and such testing provides for monitoring of the presence of the virus in a target location where such samples are collected. In other preferred embodiments for COVID-19 testing, primary samples would be saliva, buccal swab or mucus samples from individuals, and such testing provides for detection or diagnosis of subjects with active viral infections. In preferred embodiments of such testing, the present device provides for rapid, distributed testing. In preferred embodiments for this, the system provides for a test in less than 1 hour, or less than 30 minutes, or less than 15 minutes.

Other Infection Disease Applications In another preferred embodiment, the systems disclosed above can be applied to testing for and response to outbreaks of bacterial disease, such as when the food supply is contaminated by E. Coli or Salmonella. For example, in preferred embodiments, this could be an outbreak where lettuce is contaminated by E. Coli, or where ground beef is contaminated by Salmonella. such cases, in preferred embodiments testing platforms are deployed in point-of-use format to sites of food production, such as farms, fields, and processing plants. In preferred embodiments such testing platforms are also deployed in point-of-use form, as well as mobile or distributed permanent or temporary monitoring installations, to points of distribution, such as warehouses, shipping centers, or grocery stores and restaurants. In preferred embodiments, such testing platforms are deployed to the end point of consumption, such as in the home, for home-based testing. In preferred embodiments, the aggregated cloud-based analysis, including Big Data, AI and machine learning techniques, can be used to track the outbreak, and pinpoint the source.

In another preferred embodiments, the systems disclosed above can be applied to testing for sexually transmitted diseases (STDs). In this application, it is an advantage of the disclosure disclosed that such molecular electronic hybridization sensor systems can be deployed for rapid, low-cost testing in highly distributed fashion, such as in community or field clinics, or for the privacy of home use. For STDs, the causal pathogens to be detected may be parasites, such as Trichomoniasis, or fungi, such as Candidiasis (yeast infection), or bacteria such as Syphilis, Gonorrhea, or Chlamydia, or viruses such as Herpes, HPV, EBV, Hepatitis, and HIV. In such applications, the primary samples required are clinically well established, and may be a blood sample, or a swab of bodily fluids or of discharges, or from open sores. In preferred embodiments of such testing, the present device provides for rapid, distributed testing. In preferred embodiments for this, the system provides for a test in less than 1 hour, or less than 30 minutes, or less than 15 minutes. In preferred embodiments, the system provides the advantage of extreme personal privacy, with systems and test kits that can be used entirely within the home.

Experimental Demonstrations of Molecular Electronic Hybridization Sensors and Chips. Experiments that reduce these devices, methods and apparatus to practice are presented here.

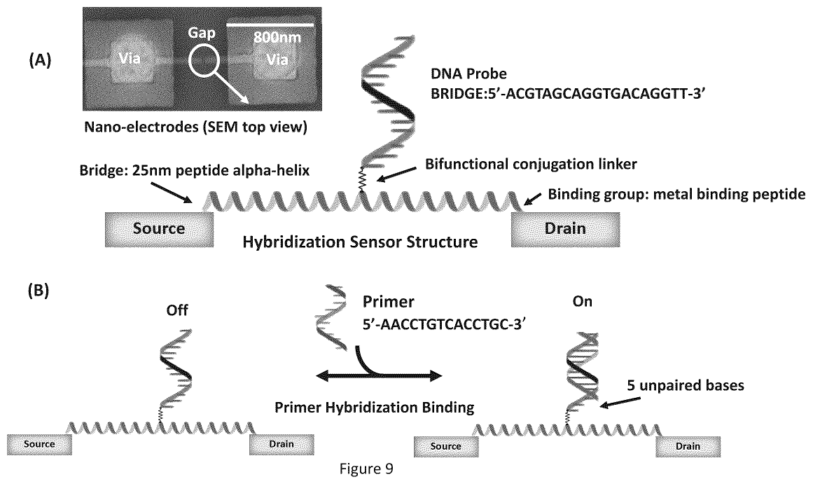

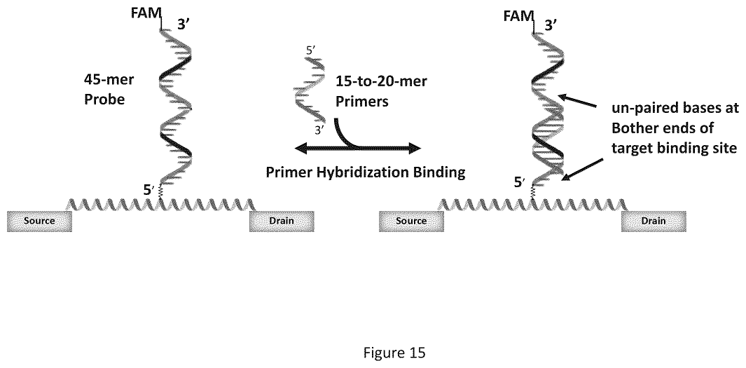

The sensor embodiments used for these experiments are shown in FIGS. 9 and 15. As shown, these consist of a DNA oligo probe, conjugated to an alpha-helical peptide bride molecule, which is bound to Ruthenium nano-electrodes. These devices are deployed on a 16 k pixel array CMOS chip device, with a 20-micron pixel pitch, fabricated in a 180 nm CMOD node, and which is a specific embodiment of the chip and pixel architecture shown in FIG. 6. Nanoelectrodes, shown in FIG. 9 in a top SEM view, were fabricated on the chip using standard methods of Electron beam Lithography, sputtering deposition of metal electrodes, and lift-off. The nanoelectrode geometry near the gap is a gap of 15 nm-20 nm, height of 20 nm, and width (looking down from above) of 50 nm. The nanoelectrodes connect to exposed Vias on the chip surface that connect the sensor into the pixel amplifier circuit as in the schematic of FIG. 6, and as shown in the SEM image in FIG. 9, showing the vias. The chip produces 8-bit digital data, at a frame rate of 1000 frames per second. The chip is mounted in a flow cell that expose a 10 uL solution volume to sensor array. the These devices are run on a custom desktop instrument platform, such as indicated be the sensor measurement instrument of FIG. 8, and data are collected on instrument, and transferred to a centralized private server, as well as the Amazon and Google clouds, for Analysis and storage, using internet and broadband wireless connections.

The specifics of the probe-bridge complex for these experiments is as follows. In all cases, the bridge molecule is a peptide that forms an alpha-helical protein structure, with primary 227 amino acid sequence SEQ1

SEQ1:
QQSWPISGSGQQSWPISGSGQQSWPISGSGAEAAAREAAAREAAAREAAA
REAAAREAAAREAAAREAAAREAAAREAAAREAAAREAAAREAAAREAAA
REAAAREAAAREACAREAAAREAAAREAAAREAAAREAAAREAAAREAAA
REAAAREAAAREAAAREAAAREAAAREAAAREAAAREAAAREAAARAGSG
QQSWPISGSGQQSWPISGSGQQSWPIS

The structure of this peptide is that of a repeat of the helix-promoting motif EAAAR, and where a central amino acid is replaced by a C to allow for Cysteine-mediated conjugation to the probe. The termini of this peptide consist of the repeats QQSWPISGSGQQSWPISGSGQQSWPISGSG, which is three repetitions of the metal binding peptide QQSWPIS, separated by short GSG spacers, and which provides for binding to the metal electrodes. In helical form, the length of this peptide is approximated 25 nm. It is used with nanoelectrode gaps in the range of 15-20 nm. This peptide was produced by bacterial protein expression of a synthetic gene encoding the peptide.

The conjugation of the hybridization probe to the bridge is done using a bifunctional cross-linker APN-BCN (Bicyclo[6.1.0]non-4-yn-9-ylmethyl (4-(cyanoethynyl)phenyl)carbamate; Sigma-Aldrich). The conjugation product is purified using a desalting spin-column, and the product peptide reacted with the hybridization probe oligonucleotide having an azide at the 5′ end, such that the 5′ end is conjugated proximal to the bridge. The resulting peptide/DNA complex was purified by size-exclusion chromatography and verified by SDS Gel electrophoresis. The relative quantities of fluorescein (for the labeled SEQ4), DNA and tryptophan were checked by UV-vis spectroscopy. In the case of the probe in FIG. 9, the attached single stranded oligo is a shorter oligo, a 19-mer DNA indicated as SEQ2:

SEQ2:
5′-ACGTAGCAGGTGACAGGTT-3′

The target for this hybridization probe in experiments is the 14-mer SEQ3, which binds leaving a 5-base gap at the Bridge-end of the probe strand:

| | |
|---|---|
| GGG | SEQ3: 5′-AACCTGTCACCTGC-3 |

In the case of the longer probe indicated in FIG. 15, this also has a fluorescein attached at the 3′ hydroxyl group, for optical detection, and the sequence is the 45-mer (also indicating the 5′ azide and 3′ FAM dye attached):

SEQ4:
/5AzideN/CGATCAGGCCTTCACAGAGGAAGTATCCTGTCGTTTAGCA
TACCC/36-FAM/

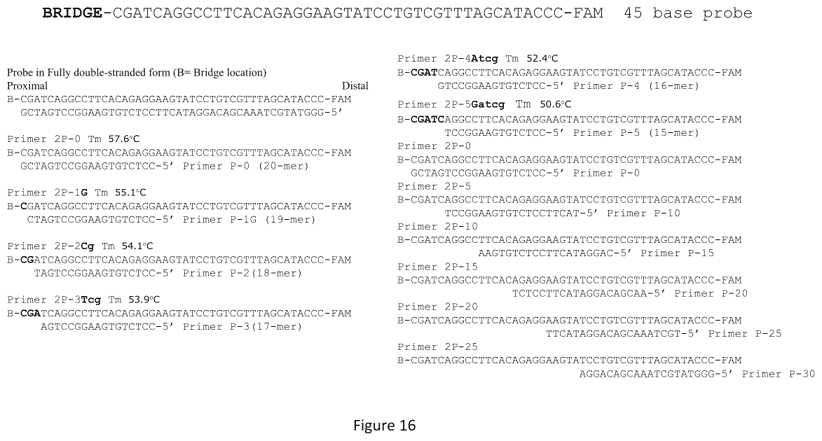

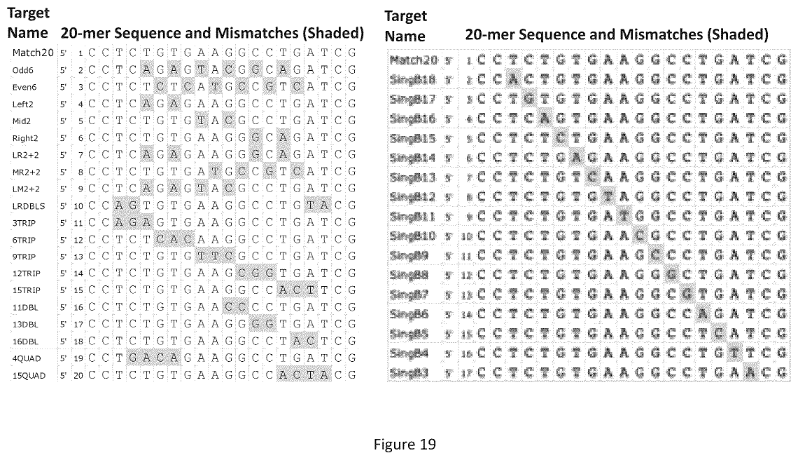

The various different primers (perfect match and with mismatches) bound to this sequence in various experiments are indicated in the table shown in FIG. 16, and FIG. 19, and the names of these many primers are indicated here.

The experimental results shown were run at room temperature, in a standard molecular biology buffer, with composition comprising 50 mM NaCl; 10 mM Tris-HCl; 10 mM MgCl₂; 1 mM DTT (pH 7.9 @ 25° C.).

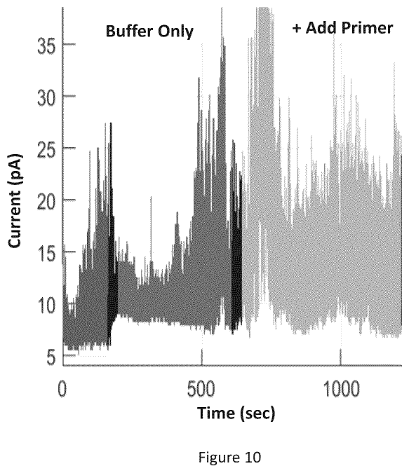

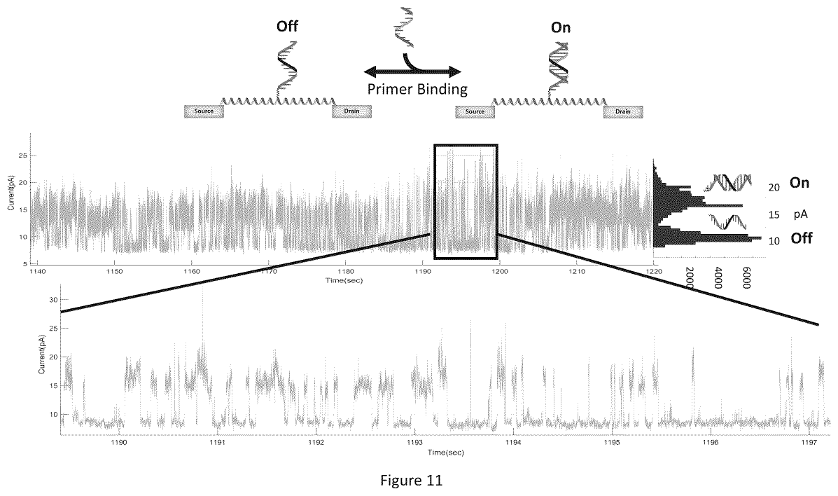

FIG. 10 shows an example signal pixel trace from the short SEQ2 probe sensor deployed on the 16 k pixel chip. In the indicated Buffer Only phase, the hybridization target SEQ3 is not present, and this is then added into the solution, typically at a 1 uM (micro-Molar) concentration. The signa trace shown covers approximately 1000 seconds of observation time. Viewed at this high level, it is clear that more and greater amplitude signal spikes occur once the primer is added. The current levels and fluctuations observed are on the scale of 20-30 pA. FIG. 11 shows a close of the signal from the pixel in the hybridization (or “primer binding”) phase, over an 8 second interval. In the close-up, two-level step ups to ˜15 pA are observed, that are interpreted as the primer-bound or “on” states indicated in the cartoon of FIG. 11, while the baseline lower current state at ˜10 pA is interpreted as the off state indicated in the cartoon. This histogram of measured current values, from the 1 kHz sampling, shows the current is distributed around these on and off levels, and provides a view of the relative time spent in each state. This trace illustrates that the sensor detects the basic hybridization of the target, distinguishable as this on-off signal train, which differs from the noise seen in the Buffer-Only phase. This demonstrates the basic functionality of this device as a target detector when viewed as a hybridization sensor.

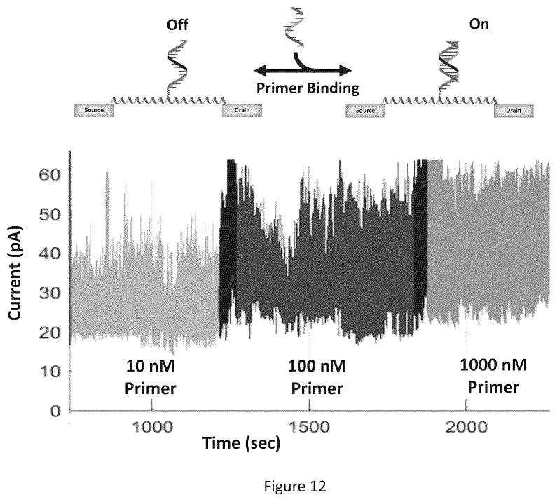

FIG. 12 shows the high-level trace response of the sensor on the chip, when the concentration of target is raised from 10 nM to 100 nM to 1000 nM (1 uM) over the course of 1500 seconds. It is clear from this high-level trace the nature of the signal is changes as concentration increases.

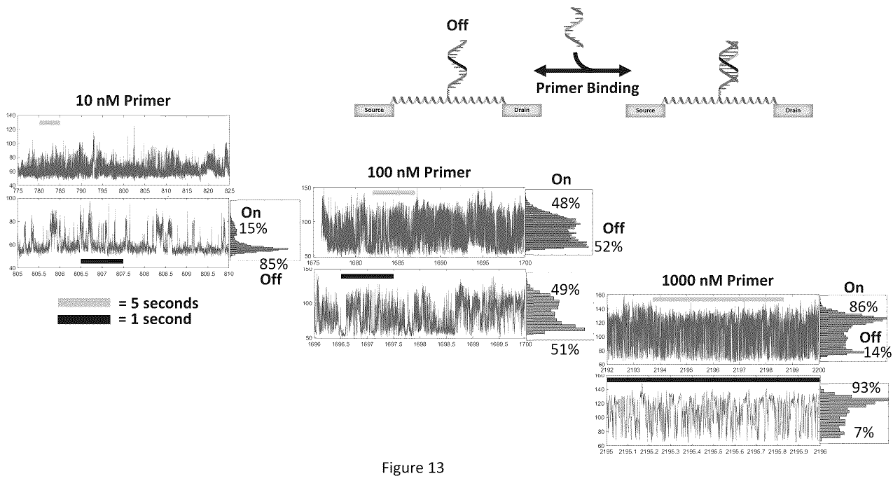

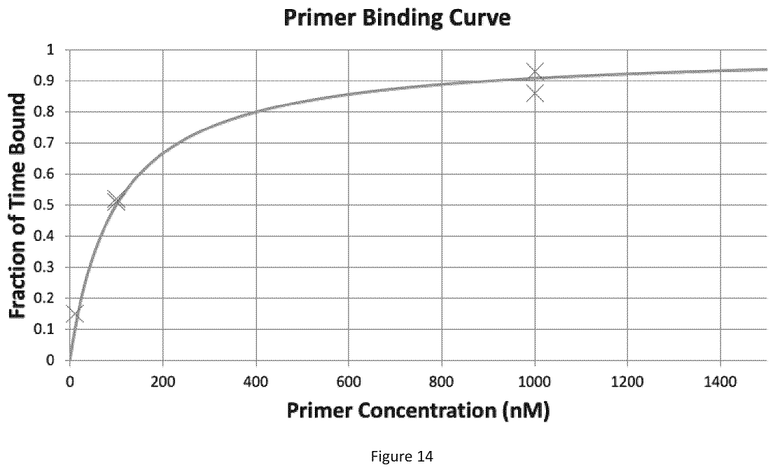

FIG. 13 shows close-ups of the signal trains in each of these phases, as well as histograms that show their relative time spent in the On and Off states. From the close-up signals, it is clear the rate of signal spikes increases in frequency as concentration increases, and also the relative proportion of on time increases, while the amplitude remains nearly constant. Thus, the variables such as pulse rate and on fraction correlate with concentration, and provide the demonstration that such a hybridization sensor provides a measure of target concentration. This demonstrates the basic functionality of this device to measure target concentration when viewed as a hybridization sensor. FIG. 14 shows the data points for on fraction versus concentration (marked as “X” in plot), and the fit of a standard exponential decay curve to such data. The curve fit shows how this sensor can be calibrated to provide an actual measure of concentration for a primary measurement such as on fraction.

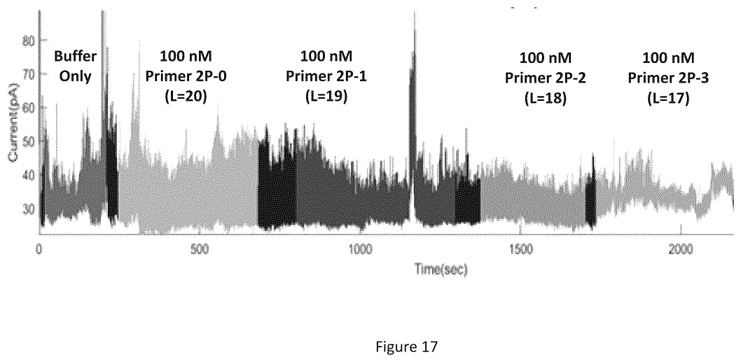

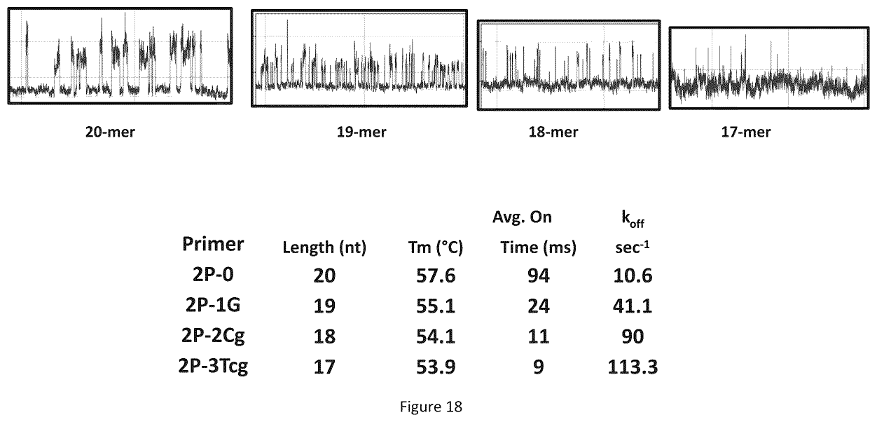

FIG. 17 shows a signal trace from one sensor on the 16 k chip, in an experiment in which the hybridization probe binds a series of progressive shorter targets, of length 20, 19, 18 and 17 bases. The targets are anchored to the same distal site, so the 3′ end of the target recedes away from the bridge as they are shortened. It can be seen in the high-level trace that the signals are diminishing as the length of target (and hence Tm) are reduced. FIG. 18 show close-ups of the signal trace in each phase, showing that as the target shortens, the amplitude of pulse drops slowly, but the average “on” time drops off more substantially, and net effect on signal trace is very clear and distinguishable. The table shown in FIG. 18 presents the length and Tm of the targets, and the average on time and corresponding off-rate. Overall, this demonstrated good sensor sensitivity, as the sensor clear detects 1-base level differences in the target (here, 1-base length differences).

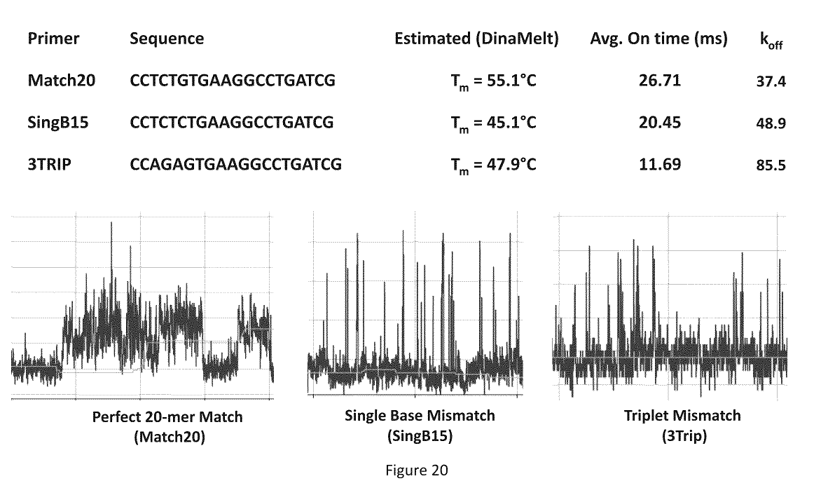

FIG. 19 shows a collection of mismatched targets, that also have 1-base differences, as well as multiple base differences, from the perfect match target shown (named as Match20). On the right side of FIG. 18 are a series of 20-mer targets with a single mismatch scanning across the length. On the left side are various 20-mer targets with multiple mismatches, from 2 to 6, spaced out or adjacent. FIG. 20 shows signal results from one base and 3 base mismatched targets relative to the signal for the perfect matched target, along with the table of respective Tm melting points, average on time, and corresponding off rate. Note that the single base mismatch makes a clear change to the signal trace, with lower “on” time as expected, as well as reduced amplitude, and the triple mismatch is even further substantially reduced in on time and amplitude. These results show that a single bas mismatch makes a substantial and detectible difference from a perfect match. This demonstrates the ability of this sensor to discriminate proper hybridization from cross-hybridization (e.g. the triple match target), which is critical to using this to sense targets in complex pools, and the single base discrimination enables applications such as SNP genotyping and strain determination, that depend on single base differences in targets.

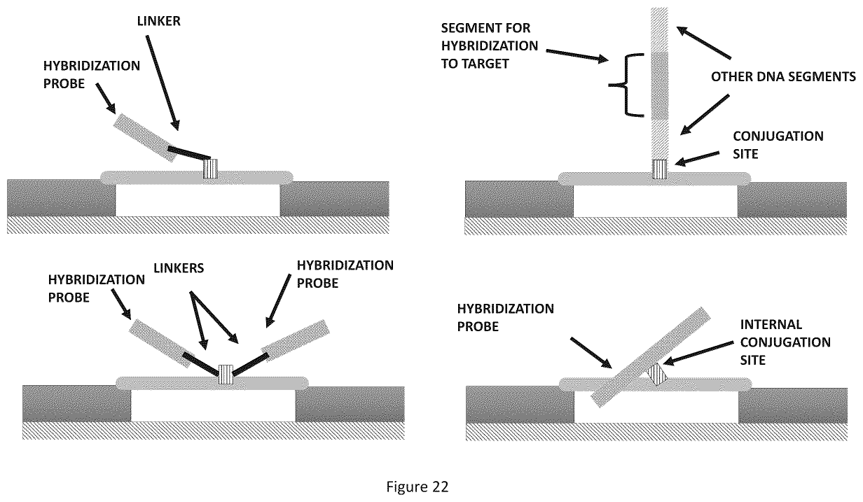

Additional Molecular Electronic s Hybridization Sensor Embodiments In these experimental examples using the sensors shown in FIGS. 9 and 15, the hybridization probe oligo is attached to the bridge at its termini, as it also is in cartoon depictions of the sensor in FIGS. 2 through 5. However, these examples and depictions are not meant to be restrictive, and in preferred embodiments, the hybridization probe may be attached at either termini, or at an internal site, such as is indicated in FIG. 22. FIG. 22 illustrates examples where the attachment is by a long linker (upper left), or multiple such linked probes, identical or distinct (lower left), or where the probe of interest with sequence specific for a target is a sub-segment of a longer DNA strand, with additional bases at one or both ends (upper right) or attachment at an internal site of the probe strand (lower right).

A suggested by FIG. 22, in preferred embodiments, the hybridization probe-containing strand may be attached in diverse manners to the bridge, also including multiple point of attachment, or having a non-linear form, such as the branched forms in FIG. 21, or a circularized form, or other diverse forms obvious to those skilled in the arts of DNA conjugation and manipulation, as long as such forms allow the portion of the strand that complements the target to be available to form a hybridized duplex with the target strand.

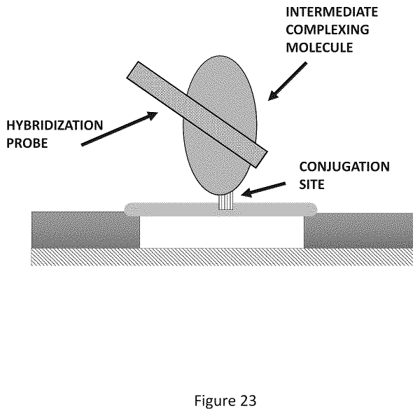

In other preferred embodiments, an additional intermediary molecule may be used to complex the hybridization probe with the bridge, such as shown in FIG. 23. Such a molecule could be a larger protein that complexes with DNA, such as a DNA binding protein, or a DNA binding enzyme such as a polymerase, ligase, restriction enzyme, CRISPR/CAS9 protein. Such an intermediate molecule may in preferred embodiments enhance the signal produced by the hybridization event.

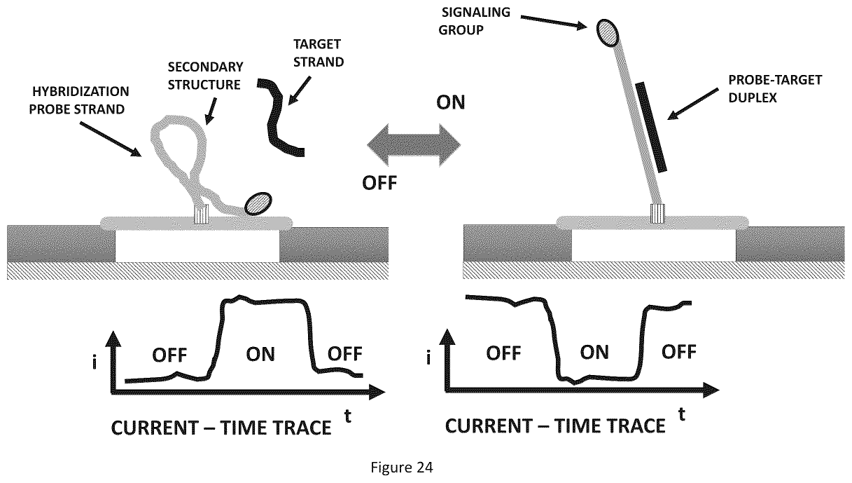

As shown in FIG. 24, in other preferred embodiments, the single stranded probe may have a preferred secondary structure with itself and or with the bridge (shown at left), that unfolds to hybridize with the target (at right). In preferred embodiments, there may also be a signaling group on the probe, which may produce a detectible signal when it changes configuration between the on and off states. In general, the secondary structure present is intended to have a melting point Tm near or slightly below that of the exact match hybridization, so that the exact match can compete with some efficiency for binding, by displacement of the secondary structure, but other fragments that do not match, have greatly reduced ability to form stable interactions with the probe. The secondary structure changes, included optionally the change in signal group location, can be used to produce a detectible signal, or a larger signal, or greater signal to noise in detecting hybridization. The secondary structure may also decrease the signals or noise from off-target strands in solution. In some preferred embodiments, the Tm of the secondary structure would be 5C-10C below the target-probe duplex Tm (C=degree Centigrade), or 1C-5C below or within 1 degree of, or 1C-5C degrees above this Tm, or 5C-10C above this Tm. In preferred embodiments, all or part of the secondary structure may be designed or induced by DNA hybridization bonding. In preferred embodiments, this is done through the use of matched and mismatched base pairing. Such secondary structure elements may include hairpin formation.

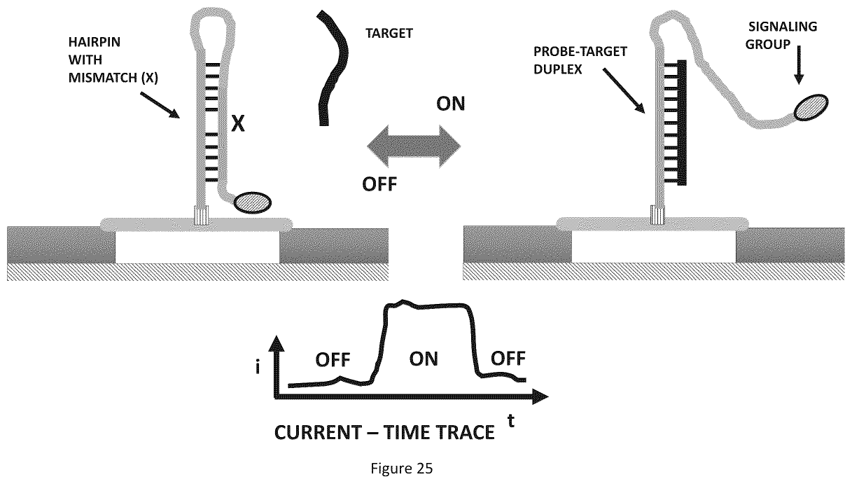

As indicated in FIG. 25, one preferred embodiment extends the probe to include a segment that forms a hairpin with one or more mismatches to the probe segment specific to the target of the hybridization. In this setting, the target molecule can bind with high stringency against unwanted off-target hybridization. The hairpin also affords the option to put a signaling group on the hairpin, such that in the hairpin form, the signal group will be held near the bridge, and in the on form bound to target, this group will be relatively far from the bridge, and more mobile, both which may contribute to a signal.

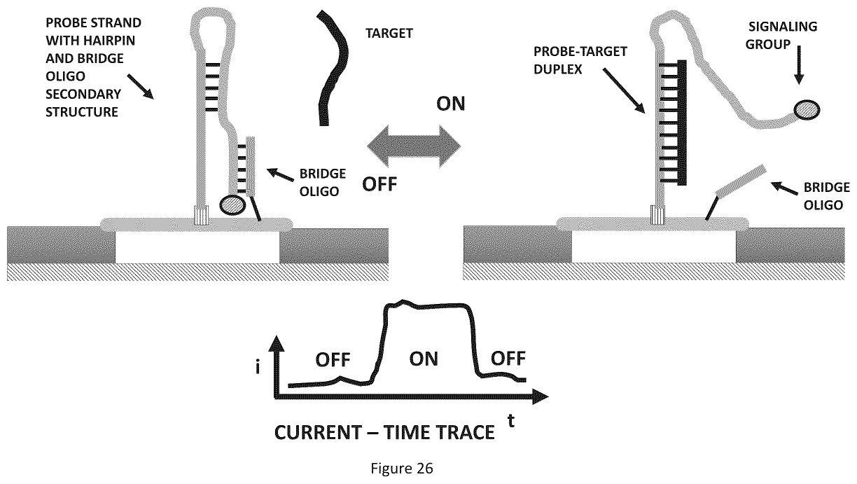

FIG. 26 shows another preferred embodiment, with a hairpin configuration and also binding to a secondary oligo attached to the bridge. This may also be used to enhance signal, either from the disruption of this secondary hybridization, or by changes in location of the signal group.

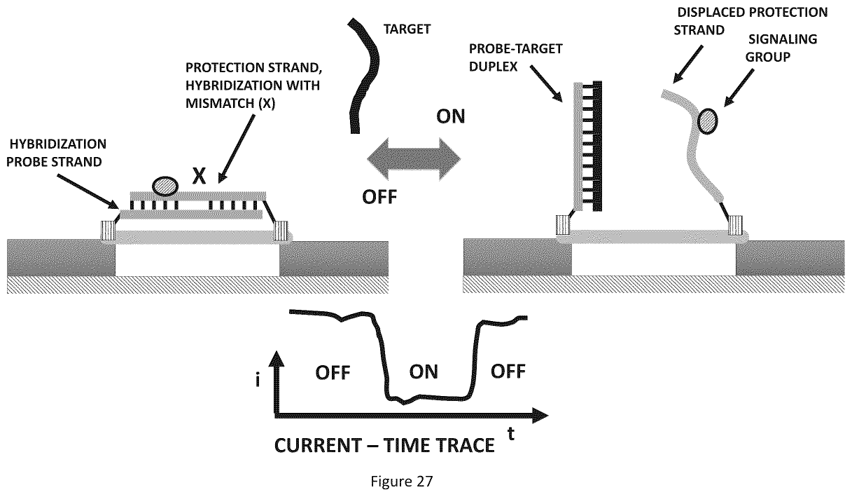

FIG. 27 shows another preferred embodiment, where a protection strand is used, which binds the target less effectively than the target, and in preferred embodiments differs by mismatches form the exact match, for finer control of this effect. The protection strand is attached and positioned as indicated, so that it creates a partial duplex with the probe that results in a parallel bridge structure to the primary bridge. This may also be enhanced by the presence of a signaling group, in preferred embodiments. Upon hybridization to target, the parallel bridge is disrupted, as is the signal group, both of which may produce a detectable signal.

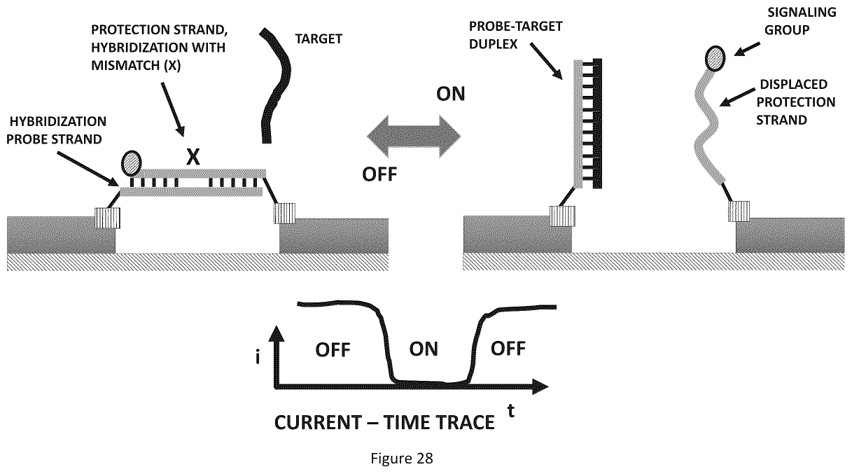

FIG. 28 shows a preferred embodiment where the bridge in FIG. 27 is the sole bridge, and so in this case the hybridization to the target completes disconnects the electrodes. This may produce a large detectable drop in current.

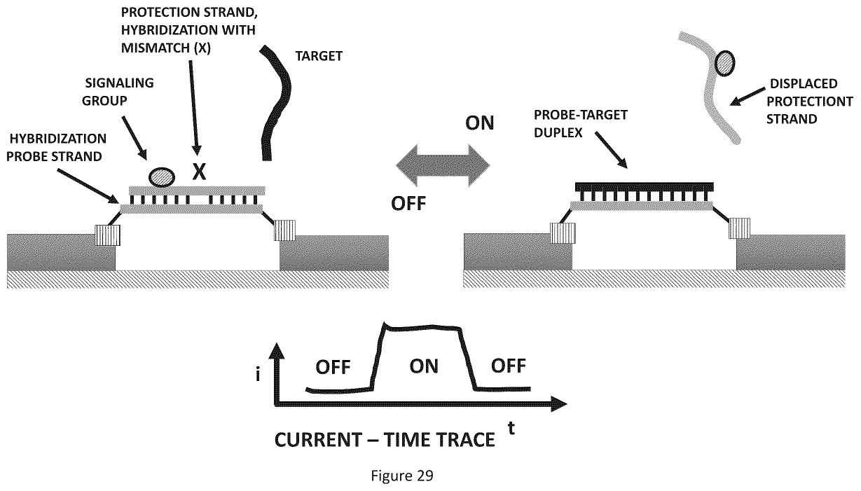

FIG. 29 shows a preferred embodiment where the bridge is formed by the protecting strand in a mismatched duplex or otherwise lower Tm duplex (relative to target Tm), optionally with a signal group on the strand, and where the protecting strand is not otherwise attached. In this case, upon hybridization to the target, the protecting stand is displaced and lost (along with the signal group), and a complete duplex is formed. These processes may result in a detectible signal.

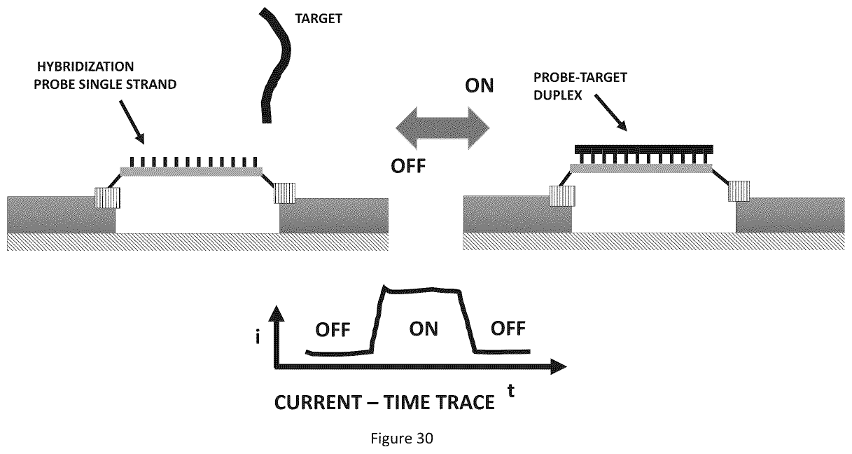

FIG. 30 shows a preferred embodiment where the single stranded hybridization probe spans the electrodes and binding to the target creates a duplex DNA bridge, which may produce a signal of a large jump to much higher current levels.

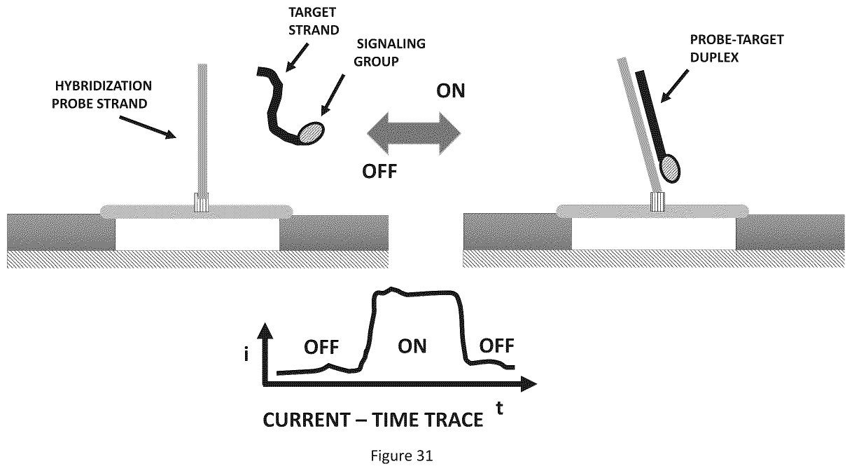

FIG. 31 shows a preferred embodiment where the target strand has an added signaling group, resulting in larger signal from the hybridization binding this group near the bridge. Such a group may be added to the sample by standard sample labelling reactions that are commonly used to attach diverse labels to DNA or RNA, and many such reactions are widely used and well known to those skilled in molecular biology or conjugation chemistry.

Note that the embodiment shown in FIG. 31, of labelling the target with a signaling group, can apply broadly to all the hybridization sensor embodiments and applications disclosed here.

There are many variations and combinations of the embodiments disclosed above that may provide useful benefits to molecular electronics hybridization sensors, methods and applications, and to one skilled in the art of molecular biology, these would be obvious variations, and these are therefore also all encompassed by the disclosure here.

### EXAMPLES

The following examples are intended to illustrate but not to limit the invention.

### Example I—Experimental Data for Tag Array Identification of Sequence Variants

### CMOS Sensor Array Chips

The proprietary CMOS sensor array chips used in this study were designed at Roswell Biotechnologies Inc. and fabricated at TSMC in Taiwan, using a 180 nm CMOS node. These chips present a 16 k (16,384) sensor pixel array. Pixels are post-processed at the IMEC foundry (Leuven, Belgium) to have the tips of Ruthenium nano-electrodes exposed on the solution-facing surface of the chip, with such electrodes fabricated using either photolithography or e-beam lithography methods. The 16 k electrodes were fabricated to have various nano-gap sizes in different ranges: 10-12 nm, 14-16 nm, 17-20 nm and 20-30 nm. Gaps of 14-20 nm were used for present experiments, and other sizes were not analyzed for present experiments. The chips were mounted in custom-built instruments to supply support to chip operations and sensor pixel data collection. The data is collected from the 16 k sensor array at a frame rate of 1000 Hz, and current measurements have 10 bits of resolution.

Alpha Helix molecular wire bridge preparation. The peptide is a helical forming sequence 242 amino acids in length, including an N-terminal FLAG sequence and metal-binding motifs at each end. In the alpha-helical conformation the length is ˜25 nm. A single cysteine resides in the middle position as the attachment point for probes using alkyne/azide click chemistry. To attach a DNA to the peptide, it was first modified using a thiol-reactive (45) 3-arylpropiolonitrile (APN)-PEG4-bicyclo [6.1.0] nonyne (BCN) (Conju-Probe, San Diego, CA) yielding a reactive bicyclo nonyne alkyne on the peptide. Typically, 100 μL of peptide solution (3 to 4 mg/mL in PBS) was first mixed with freshly prepared DTT or TCEP (2 mM final) and left at room temperature for an hour. Then the APN-BCN reagent dissolved in DMSO (1 M stock), is added to a final concentration of 0.01 M and mixed thoroughly by pipetting. The reaction is left at 4° C. for a minimum of 48 hours. The excess APN-BCN is removed by size-exclusion chromatography. The purified peptide-BCN is stored at −20° C. until needed. Further click reactions are done using oligos designed with azide to obtain the bridges used in this study. The reaction of BCN-azide was performed in PBS at molar excess of the oligo-azide to purified peptide-BCN prep. The final reaction was further chromatographically purified to more than 95%. The oligos were blocked at the free 3′ end with a fluorescent dye (FAM or Cy3 to help detection of peptide on SDS-PAGE). A gel shift on SDS-PAGE confirmed the bridge conjugation to oligos.

Sequences of oligos used in this study as DNA probes:

17mer-
TACGTGCAGGTGACAGG/FAM/
45mer-
CGATCAGGCCTTCACAGAGGAAGTATCCTCGTTTAGCATACCC/FAM/

### DNA Oligo Binding Experiments

All oligo binding experiments performed in a buffer 50 mM Tris HCl pH 7.5, 4 mM DTT, 10 mM KCl and 10 mM SrCl2 (Buffer A). Primer P-3 binds with its 3′ terminus 3 nucleotides away from the bridge; the sequence is 5′-CCTGTCACCTGCAC, complementary to the 17mer.

### DNA Melting Temperature Experiments and Tm Estimation

All temperature melt experiments were using the 45mer probe-peptide bridges. The two oligos used in this analysis are 2P-0: CCTCTGTGAAGGCCTGATCG and 2P-5: CCTCTGTGAAGGCCT. The temperature changes were controlled by the software interface that communicates with a Peltier device sitting attached to the chips. The temperature ramps were recorded as ignore and resume phases while every two-degree step were recorded continuously for four minutes of data collection stabilized at the temperature desired.

### DNA Match-Mismatch Binding Experiments

For assessing the binding kinetics for match and mismatch oligos following oligos designed against the 45mer probe bridge.

Exact Match 5′—CCTCTGTGAAGGCCTGATCG, 1 Mismatch 5′-CCTCTCTGAAGGCCTGATCG, 2 Mismatch 5′—CCTCTGTGAACCCCTGATCG, 3 Mismatch 5′-CCAGAGTGAAGGCCTGATCG. Targets (all 20-mers) were added separately, and binding kinetics monitored to tabulate fraction bound and other parameters.

### Results

The experiments below involve attaching bridge molecules to metal electrodes on chips with nanoelectrode spacings in the desired ranges of 15 nm to 20 nm. The molecular bridge used here is a 25 nm peptide with specific metal binding sequences at both ends. The end groups allow it to self-assemble onto the electrodes under proper conditions. An “active bridging” protocol uses electrical forces to attract the bridge to the electrodes, to radically accelerate and enhance this assembly process, allowing assembly to be completed in seconds. Specifically, using dielectrophoretic trapping readily shortens this time to 10 seconds, and also allows working at much lower input concentrations of bridge molecules. The dielectrophoretic trapping protocol relies on the application of an AC voltage (here 100 KHz, 1.6 V peak-to-peak). The DC current on sensor the after bridging is compared with the value prior, to assess the jump in current indicative of successful bridging. A population of sensors showing substantial bridging current increases is thereby observed, typically over 10% of all available pixels on the chip, indicating the presence of the 25 nm peptide bridge spanning the electrode gap.

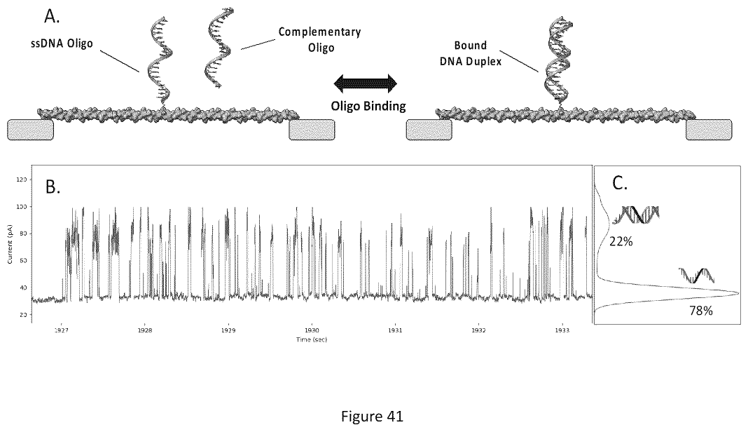

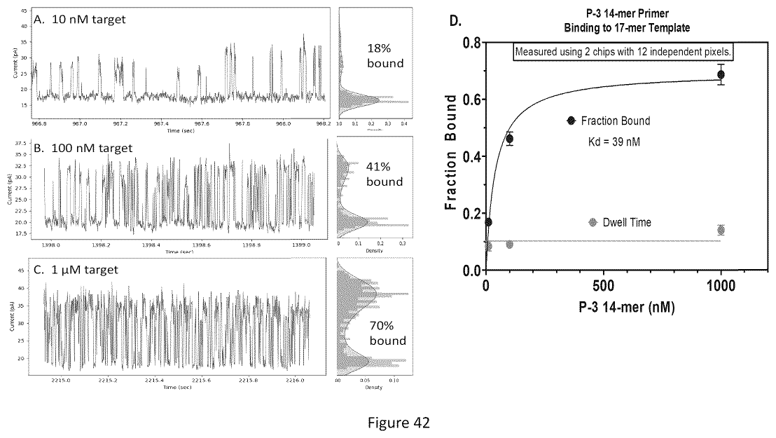

DNA-DNA hybridization binding. The probe molecule attached to the molecular wire bridge for the work here is a single-stranded 17-mer DNA oligonucleotide with a specific sequence (FIG. 41). The specific binding target for this probe is the complementary sequence DNA oligomer. This DNA probe was conjugated the bridge molecule in a precision site-specific manner to the central amino acid on the bridge, using conventional alkyne/azide copper-less click chemistry and purified by HPLC for application on chip. Once the bridge molecules (with probe) are attached to the electrodes of the chip, baseline current is measured, which is typically steady for hundreds of seconds, with a range of 2-3 pA when a voltage of 700-1000 mV is applied. While continuing to monitor chip current, the “target” oligonucleotide is added at a particular concentration. As shown in FIG. 42, the sensor on chip responds to the presence of the target in solution with current pulses of that can be interpreted as individual DNA binding events with the DNA strand attached to the sensor, in a dynamic equilibrium between bound and un-bound states. Control experiments (FIG. 41) revealed that this binding changes with the target concentration and temperature as would be expected for DNA-DNA binding. It is important to note that while various summary statistics described below provide the classical kinetic rate parameters, the complete time trace contains extremely rich information about the molecular interaction, such as possibly indications of multiple conformations and partial interactions.

Analysis of single-molecule binding data using Hidden Markov Models (HMM) Hidden Markov Models (HMM) are signal processing methods known to be well-suited to the analysis of such timeseries measurements, based on their extensive use in speech recognition. In the present case, the trace is fit to a 2-state HMM, that has also in the past been successfully applied to data from single-molecule biophysics experiments, particularly those using nanopores and enzymes. The HMM assigns the “hidden” bound and unbound states of the sensor oligo to segments of the signal representing two different current levels that correspond to either the “unbound” bridge state (low current) or the “bound” bridge state (higher current). In this case, the unbound state is identified as a low-current range (˜30 pA) and the bound state is identified as the high current range (50 pA-70 pA). The key fundamental parameters that can be extracted from the HMM segmented signal trace are the individual waiting times between binding events, τ₀, and the individual dwell times spent bound, τ₁.

While these times are ideally exponentially distributed, note the complete empirical distributions also potentially contains richer information about the more complex nature of the binding interactions. Knowing these fundamental event duration times allows calculation of the kinetic rates that characterize such a binding reaction. The duration of such events are random variables, with an exponential probability density distribution,

$$y = ( \frac{1}{\overline{τ}} ) {e}^{-} ( \frac{t}{\overline{τ}} ) ) ,$$

where t is an event duration time, τ̅ is the mean (average) of all the state durations measured, and the apparent rate constant for the reaction is

$$k = ( \frac{1}{\overline{τ}} ) .$$

The key kinetic rate parameters are the off rate, k_{off}, which is computed from the totality of dwell times,

$${k}_{off} = ( \frac{1}{{\overline{τ}}_{1}} ) ,$$

and the on rate, kₒₙ, computed from the waiting times,

$${k}_{on} = ( \frac{1}{{\overline{τ}}_{0}} ) .$$

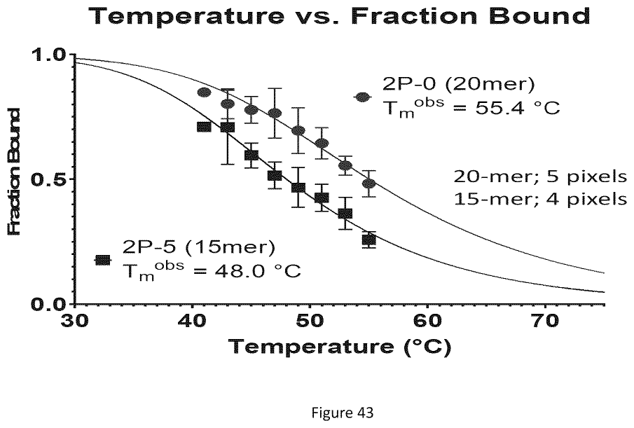

In addition, the total fraction of time spent bound is another convenient summary statistic that is readily related to the concentration of the target in solution, and is also conveniently related to overall classical binding affinity of the interaction, K_{d}, which here is defined at the single-molecule level as the target concentration at which the single probe molecule spends equal time bound and unbound. By formula, the fraction of time that the probe is bound (denoted “Fraction Bound” in all figures) to the target molecule is given by the sum of all the τ₁ periods divided by the total of both the τ₁ and τ₀ periods: f_{b}=τ₁/(Στ₁+Στ₀). Note that as target concentration increases, the mean waiting times will decrease in proportion, while dwell times should remain constant being a property only of the interaction, and thus the fraction of time bound is expected to scale with molar concentration, [c], like f_{b}=1/(1+[c]/K_{d}), where K_{d} is the empirical binding affinity concentration. Note that K_{d} can also be visualized as the inflection point in a titration curve plot of Fraction Bound vs. Target Concentration, as shown throughout this report. Also note net amount of time spent in the bound and unbound states can be conveniently visualized using vertical histograms of the measured current values in a signal segment (sampled at 1 kHz) as shown to the right of the traces in FIGS. 42B and 43. Again, the details of these histograms may also contain of richer information about the interactions, such as indications of additional bound states conformations.

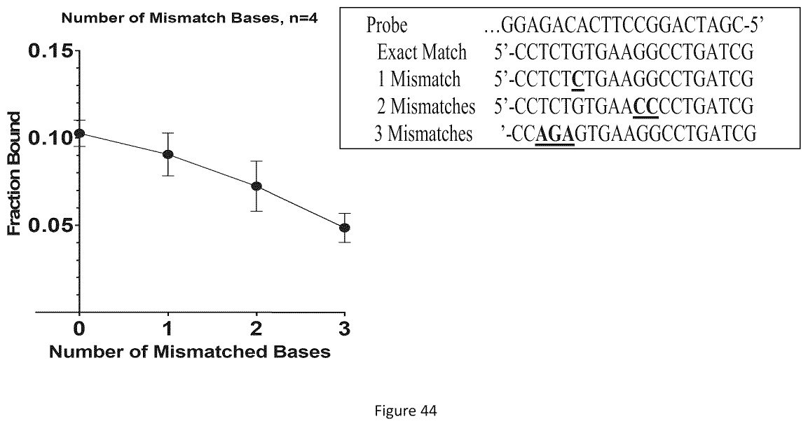

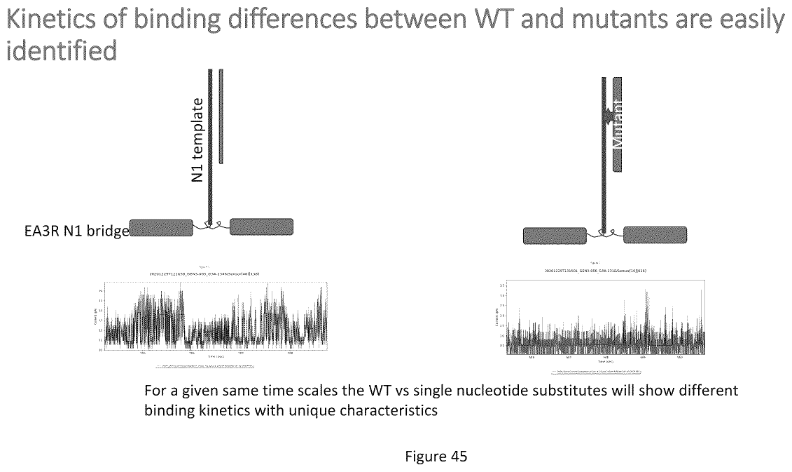

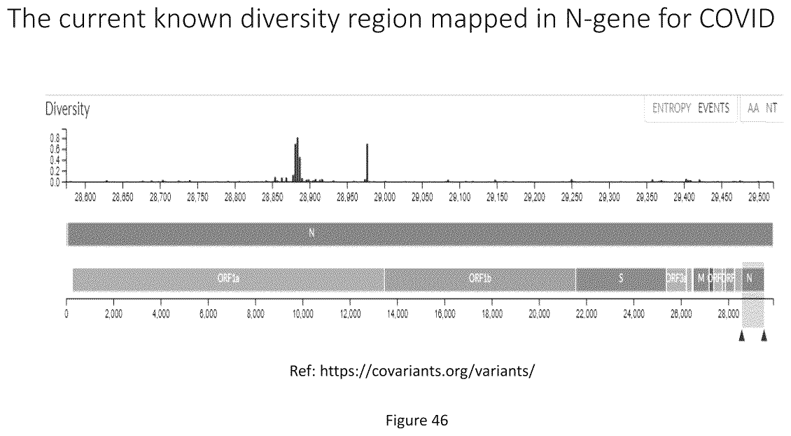

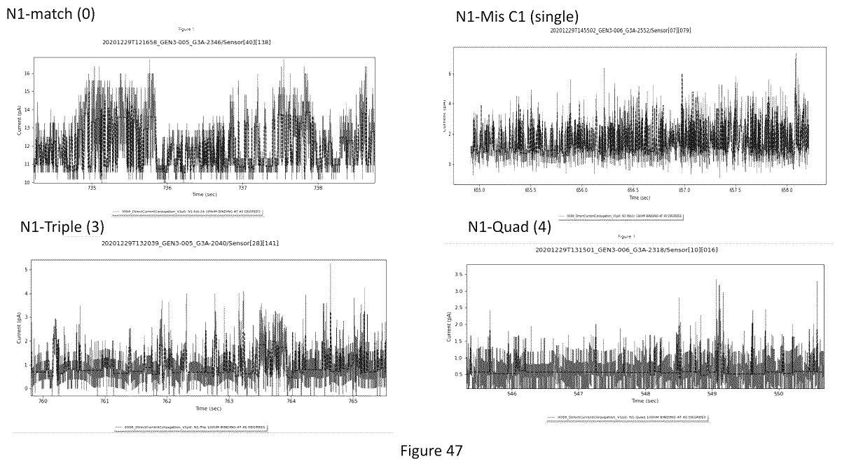

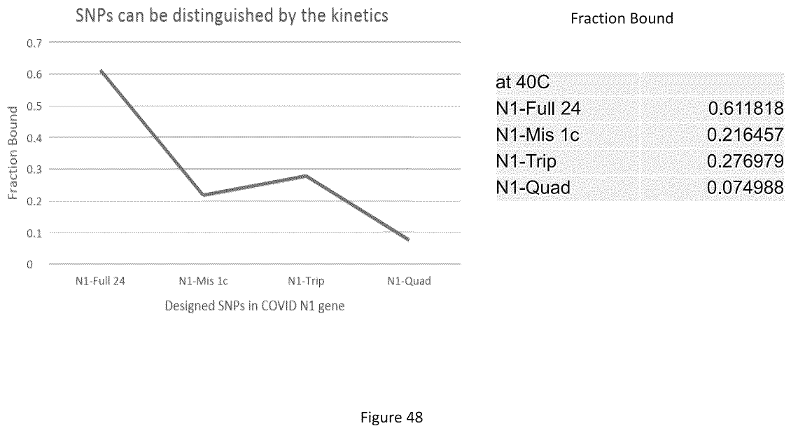

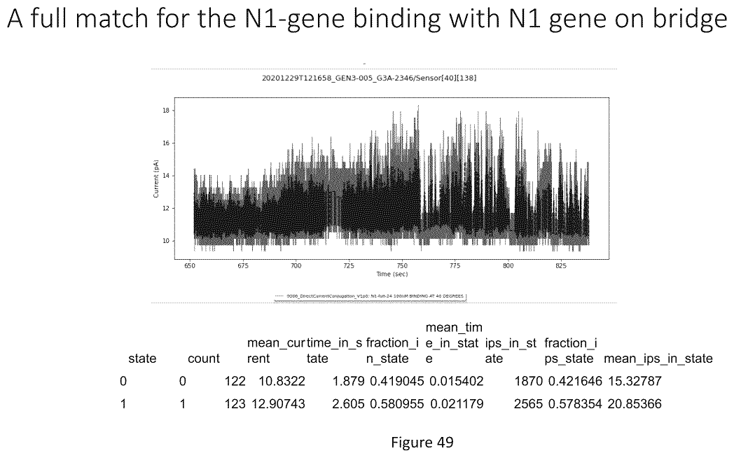

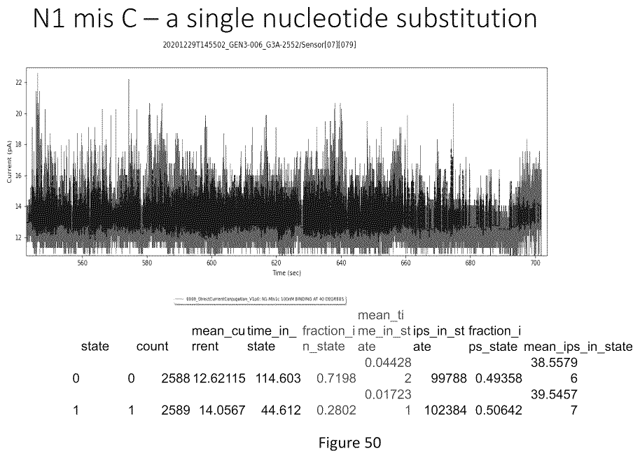

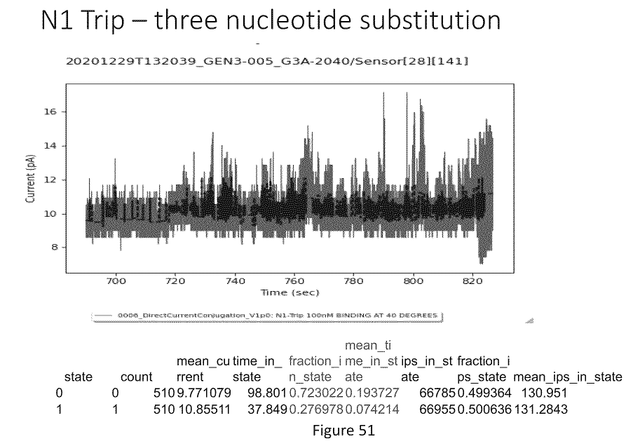

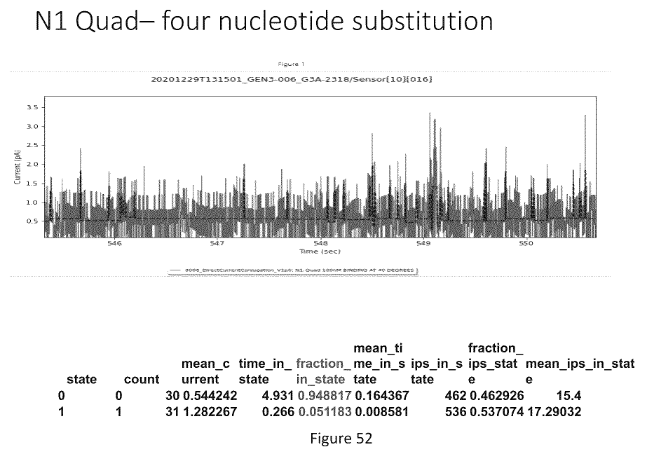

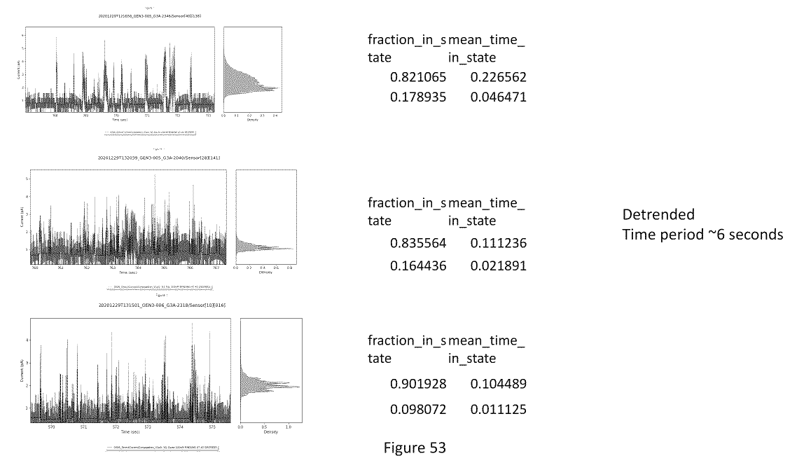

Single Molecule Thermodynamics: Melting Curves Another application of this same type of assay is to determine the melting temperature (Tₘ) of the DNA duplex, which is defined here at the single-molecule level here as the temperature at which the probe DNA molecule spends equal amounts of time in the bound and unbound states. This is directly observable in the single molecule binding traces. As shown in FIG. 44, measuring of fraction bound at about eight temperatures allows fitting of the data to a classical DNA hybridization melting curve. Here measured temperature values shown are temperatures taken on the chip die itself, using on-chip temperature sensors, as a Peltier-drive heating plate in direct contact with the die is used to set to different temperatures (and allowing time to equilibrate to each new temperature) in succession in one chip experiment. From these curves it is clear that a 20-mer target oligo melts off at a higher temperature than 15-mer segment of this 20-mer oligo, as expected. Determination of Tₘ serves to validate that this is measurement of the DNA-DNA hybridization binding reaction as intended, and also has practical value in selecting the experimental operating temperature for using such a probe to make concentration measurements. As is done classically, this single-molecule melting curve be used to increase the signal-to-noise for detecting the target of interest, or to characterize details of targets that contain mismatches.

Mismatch Sensitivity The single-molecule binding probe signal trace contains rich inform about the binding reaction, and is also highly sensitive to the specific binding target. This can be illustrated in fine detail for DNA oligo binding by looking at the impact of single-base mismatches in the target oligo sequence. As shown in FIG. S4, several variants of the exact match 20-mer target sequences were made, introducing from 0 to 3 mismatched bases. Results from applying these variant targets to the probe, and measuring bound fraction, are shown. With each additional mismatch, the bound fraction decreases. These differences are readily measured and can be further magnified by performing the measurements at a temperature nearer to the Tₘ of the matched target, or by performing a temperature melting curve as in FIG. 44. The binding dwell time is also reduced by the presence of mismatches, and can provide a comparable—and independent-indicator (data not shown). This sensitivity to mismatches can have applications for DNA binding assays in which sequence variants relative to a reference sequence probe might be of interested, such as in detecting novel strains of a viral genome, or detecting diverse somatic mutations in a cancer genome, or in detecting SNP genotype variants.

### Example II—COVID N1-SNPs and Binding Kinetics

The buffer used for the experiment was 50 mM Tris 10 mM KCL and 4 mM DTT pH 7.5Temps: 30, 40 and 50. Chips: Ruthenium Bridge: N1Br21; EA3R-/5AzideN//iCy3/CCGCATTACGTITGGTGGACC Voltage: 0.7 V

Primers: N1-full-21, N1-double, N1-Trip, N1-Quad, N1-Mis1a, N1-Mis1b, Ni-Mis1c, N1-Mis-ends at 100nMResults: bufferTK-10 at pH 8.0bridge N1-full 24 EA3R at 20 nM

Dry baseline was recorded for few seconds wet baseline in bridging solutions itself. Bridging conditions at IV follow this 100 kHz for 10 sec, 60s at 1 MHz-odd, 30s at 1 MHz-even, 10s 1 MHz even. The oligonucleotides used in binding kinetics assays are described below.

TABLE 14

| | |
|---|---|
| N1-Full-24 | ggtccaccaaacgtaatgcggggt |
| N1-full-21 | ggtccaccaaacgtaatgcgg |
| N1-Mis 1a | ggtcGaccaaacgtaatgcgg |
| N1-Mis 1b | ggtccaccaaacgtaatCcgg |
| N1-Mis 1c | ggtccacGaaacgtaatgcgg |
| N1-Mis-ends | ggtcGaccaaacgtaatCcgg |
| N1-Double | ggtccaccaaacgtTTtgcgg |
| N1-Trip | ggtcGTGcaaacgtaatgcgg |
| N1-Quad | ggtccaccaaaGCATatgcgg |

In this experiment, N1Br26/5AzideN//iCy3/ccccgcattacgtttggtggaccctc is used as the probe containing the coding for the N1 gene of COVID that is tethered on the peptide bridge that act as a sensor that connect the two electrodes (see the diagrammatic representation in the following file). A series of single, double, triple and Quadruple mutants are designed into the primers (the nucleotides substitution is shown in CAPs).

TABLE 15

| | |
|---|---|
| N1Br31 | /5AzideN//iCy3/gcaccccgcattacgt |
| | ttggtggaccctcag |
| N1Br26 | /5AzideN//iCy3/ccccgcattacgtttg |
| | gtggaccctc |
| N1Br21 | /5AzideN//iCy3/ccgcattacgtttggt |
| | ggacc |
| N1Br17 | /5AzideN//iCy3/gcattacgtttggtgg |
| | a |
| N1Br14 | /5AzideN//iCy3/gcattacgtttggt |
| N1-Full-24 (3′-5′) | 3′-TGGGGCGTAATGCAAACCACCTGG-5′ |
| N1Br31 | /5AzideN//iCy3/gcaccccgcattacgt |
| | ttggtggaccctcag |
| RPAN1-72F | 5′-GACCCCAAAATCAGCGAAATGCACCCCG |
| | CATTAC-3′ |
| RPAN1-72R | 5′-TCTGGTTACTGCCAGTTGAATCTGAGGG |
| | TCCACC-3′ |
| RPAN1-94F | 5′-GTCTGATAATGGACCCCAAAATCAGCGA |
| | AATGC-3′ |
| RPAN1-94R | 5′-TGCGTTCTCCATTCTGGTTACTGCCAGT |
| | TGAATC-3′ |

Table 15 above shows exemplary EA3R bridges tethered to study the SNP kinetics.

TABLE 16

| | |
|---|---|
| N1-Full-24 | ggtccaccaaacgtaatgcggggt |
| N1-full-21 | ggtccaccaaacgtaatgcgg |
| N1-Mis 1a | ggtcGaccaaacgtaatgcgg |
| N1-Mis 1b | ggtccaccaaacgtaatCcgg |
| N1-Mis 1c | ggtccacGaaacgtaatgcgg |
| N1-Mis-ends | ggtcGaccaaacgtaatCcgg |
| N1-Double | ggtccaccaaacgtTTtgcgg |
| N1-Trip | ggtcGTGcaaacgtaatgcgg |
| N1-Quad | ggtccaccaaaGCATatgcgg |

Table 16 above shows exemplary SNPs designed into the primers to demonstrate Primers designed from Full match to four nucleotide substitutions that can be identified using kinetics of binding.

### EXEMPLARY EMBODIMENTS

Embodiment 1: A molecular electronics sensor configured to detect a tag from a tag set, using any of the hybridization sensors disclosed, and comprising the reverse complement of the tag.

Embodiment 2: A tag reporter assay, producing a tag reporter, and using the sensor of Embodiment 1 to detect the tag.

Embodiment 3: A molecular electronics sensor array chip configured to detect the tags from a tag set.

Embodiment 4: A multiplex tag reporter assay, producing reporter tags from a given tag set, and using the sensor array chip of Embodiment 3 to detect the reporter tags.

Embodiment 5: The multiplex assay of Embodiment 4, for the detection of target DNA fragments, using linear detection probes as in FIG. 34, or using circularizing detection probes as FIG. 36.

Embodiment 6: The multiplex assay of Embodiment 4, for the detection of allele-specific target DNA fragments, using linear allele-specific detection probes as in FIG. 35, or using circularizing allele-specific detection probes as FIG. 37.

Embodiment 7: The multiplex assay of Embodiment 4, used for multiplex detection of ligand binding reactions as in FIG. 38.

Embodiment 8: The multiplex assay of Embodiment 4, used for multiplex detection of ligand binding interactions, and using bipartite tags as shown in FIG. 39, with such tags coming from a bipartite tag set.

Embodiment 9: Tags sets designed to have good properties as disclosed, and functionally screened for good performance on the molecular electronic sensor array chip format of Embodiment 3.

Embodiment 10: Tag sets of Embodiment 9, where such tags in the set number up to 10, up to 100, up to 1000, up to 10,000, up to 100,000, up to 1,000,000, up to 10,000,000, or up to 100,000,000.

Embodiment 11: The tag sets of Embodiment 9, where the set are designed to be Tm matched near 30° C., near 40° C., near 50° C., near 60° C., near 70° C., near 80° C., near 90° C., or near 100° C., relative to a given

Embodiment 12: The tag sets of Embodiment 9, where the set are designed to have the unwanted reaction melting points lower than the target tag Tm by at least 10° C., or 20° C., or 30° C., or 40° C., or 50° C., relative to a given hybridization assay condition.

Embodiment 13: The tag sets of Embodiment 9, where the tags are bipartite tags.

Embodiment 14: Tag sets containing modifications to enhance sensor performance, such as modified nucleotides, nucleotide analogues, or signal enhancing chemical groups or labeling groups which may be attached to DNA oligos, either the tags themselves, or to the tag complement, as well as the design and methods of using such tag sets.

Embodiment 15: A method for genotyping analysis of a DNA sample, consisting of using the tag reporter assays and tag sensor arrays, as disclosed.

Embodiment 16: A method for determining which strain of a pathogen is present in a DNA sample, consisting of using the tag reporter assays and tag sensor arrays, as disclosed.

Embodiment 17: A method for performing gene expression analysis of a sample, for a given set of genes, consisting using the tag reporter assays and tag sensor arrays, as disclosed.

Embodiment 18: A method for pathogen detection, by applying the methods for detecting the presence of a target DNA segment above, consisting of using the tag reporter assays and tag sensor arrays, as disclosed.

Embodiment 19: A process for the applications of infectious disease detection, environmental monitoring, screening, or diagnosis, consisting of collecting a sample from the environment or a subject, providing this sample to a sample preparation system that prepares an extracted DNA sample, applying a multiplex tag reporter assay, providing this sample to the molecular electronics hybridization sensor chip system for the tag set, and processing the hybridization data to determine the presence of the pathogens of interest, as in and producing a final report out of detection or non-detection, using either local analysis, reporting and storage of data at the point of measurement, or remote or cloud-based analysis, reporting and storage of results.

Embodiment 20: The process of Embodiment 19, used for pandemic viral disease testing and monitoring, such as for COVID-19.

Embodiment 21: An apparatus and kit for testing for COVID-19 in environmental or human subject samples, based on the methods and processes of the above embodiments.

Embodiment 22: The process of Embodiment 19, for the detection of Sexually Transmitted Diseases.

Embodiment 23: An apparatus and kits for testing for STDS, based on the methods and processes of the above embodiments.

Embodiment 24: The process of the above embodiments, used for the detection of food borne pathogens.

Embodiment 25: An apparatus and kits for testing for food born pathogens, based on the methods and processes of the above embodiments.

1. A molecular sensor comprising:
a source electrode;
a drain electrode spaced apart from the source electrode by a sensor gap;
a gate electrode, wherein the source, drain and gate electrodes cooperate to form an electrode circuit;
a bridge bridging across said sensor gap coupling the source and drain electrodes; and
a probe coupled to the bridge, wherein the probe comprises a nucleic acid and interaction of the probe with a target nucleic acid is detectable by monitoring at least one parameter of the electrode circuit.

2. The molecular sensor of claim 1, wherein the nucleic acid probe is single stranded.

3. The molecular sensor of claim 1, wherein the nucleic acid probe is single stranded and the target nucleic acid comprises a single stranded nucleic acid.

4. The molecular sensor of claim 1, wherein the molecular sensor comprises more than one nucleic acid probe coupled to the bridge and each nucleic acid probe further comprises a tag reporter sequence.

5. The molecular sensor of claim 1, further comprising a plurality of molecular electronics sensors configured in an array of sensor pixels on a CMOS chip, each molecular electronics sensor configured in a pixel.

6. The molecular sensor of claim 3, wherein the nucleic acid probe comprises a single stranded oligonucleotide.

7. The molecular sensor of claim 3, wherein the single stranded nucleic acid probe comprises a signaling group.

8. The molecular sensor of claim 3, wherein the single stranded nucleic acid probe forms a secondary structure with itself to form one or more loops or circles.

9. The molecular sensor of claim 1, wherein the bridge comprises one bridge molecule bridging across said sensor gap coupling the source and drain electrodes.

10. The molecular sensor of claim 1, wherein the bridge comprises two single stranded nucleic acid molecules bridging across said sensor gap wherein one bridge molecule is coupled to the source electrode and the other bridge molecule is coupled to the drain electrode and hybridization of the two together completes the electrode circuit.

11. The molecular sensor of claim 1, wherein the bridge comprises a peptide alpha helix.

12. The molecular sensor of claim 1, wherein bridge comprises a peptide alpha helix and the probe is a ssDNA that is conjugated to the protein alpha helix bridge by a linker.

13. The molecular sensor of claim 1, further comprising a decoding probe coupled to nucleic acid probe and the decoding probe maps the location of nucleic acid probe within a sensor pixel array on the molecular sensor.

14. The molecular sensor of claim 1, wherein one or more mismatch in the nucleic acid sequence of the nucleic acid probe with a target nucleic acid upon hybridization is detectible.

15. A method of detecting a target nucleic acid, the method comprising:
on a molecular sensor, hybridizing a target nucleic acid with a probe molecule comprising a targeting sequence complementary to the target nucleic acid;
observing perturbations in a measured electrical parameter of the sensor circuit during hybridization of a target nucleic acid with a probe; and
relating characteristics of the perturbations to the presence of the target nucleic acid.

16. A method of claim 15, wherein the target nucleic acid comprises a segment of a pathogen genome.

17. A method of claim 15, used for infectious disease detection, environmental monitoring, screening or diagnosis.

18. A method of claim 15, used for detection of COVID-19 in environmental or human subject samples.

19. A method of claim 18, used for detection of COVID-19 wherein the target sequence comprises at least a portion of a COVID-19 sequence described herein.

20. A method of claim 19, wherein the portion of COVID-19 is between 4 and 18 contiguous nucleotides, between 5 and 15 contiguous nucleotides, between 6 and 12 contiguous nucleotides of the entire sequence.
